(19) United States
(12) Patent Application Publication
(10) Pub. No.: US 2026/0266798 A1
(43) Pub. Date: Sep. 10, 2026

(54) SYSTEMS AND METHODS REGARDING PEAK EXPLORATION AND ARTIFICIAL TISSUE RESPONSE INCLUDING FOR CONTRACTION TYPE CLASSIFICATION OF ENGINEERED TISSUE

(71) Applicant: VALO HEALTH, INC., Lexington, MA (US)

(72) Inventors: Nathan Bays, Boston, MA (US); Brian Schriver, Boston, MA (US)

(21) Appl. No.: 19/166,523

(22) PCT Filed: Apr. 5, 2024
(86) PCT No.: PCT/US2024/023275

§ 371 Date: Sep. 18, 2025

Related U.S. Application Data
(60) Provisional application No. 63/457,574, filed on Apr. 6, 2023.
(60) Provisional application No. 63/466,105, filed on May 12, 2023.
(60) Provisional application No. 63/505,257, filed on May 31, 2023.
(60) Provisional application No. 63/526,566, filed on Jul. 13, 2023.

(51) Int. Cl. G01N 33/483; G01N 21/63

(52) U.S. Cl. CPC G01N 33/4833; G01N 21/63; G01N 2223/305; G01N 2223/6126

(57) ABSTRACT

Systems and methods are described herein for detecting spontaneous tissue contractions. In one example aspect, a functional response waveform may be processed, which comprises obtaining a first waveform comprising at least one contraction response and at least one relaxation response of an engineered tissue. A predicted contraction type may be determined for of a plurality of contraction types (e.g., single and double types) for the first waveform. A model may be fitted to the first waveform based on the predicted contraction type, where the model parameterizes growth of the at least one contraction response of the engineered tissue independently of growth of at least one relaxation response of the engineered tissue. A second waveform may be generated from the model as fit to the first waveform such that the second waveform comprises a noise filtered representation of the first waveform.

This application claims the benefit of U.S. Provisional Application No. 63/457,574 (filed on Apr. 6, 2023); U.S. Provisional Application No. 63/466,105 (filed on May 12, 2023); U.S. Provisional Application No. 63/505,257 (filed on May 31, 2023); and U.S. Provisional Application No. 63/526,566 (filed on Jul. 13, 2023). The entirety of each of the foregoing provisional applications is incorporated by reference herein.

### BACKGROUND

Tissue behavior can be measured and modeled as a waveform of tissue response (e.g., functional response or contractile force) versus time. The resulting functional response waveform is a fundamental and valuable unit of representation and encoding for tissue data. This representation and encoding of tissue behavior as waveforms provides a rich substrate for downstream analysis. However, existing approaches to featurization of such waveforms requires the extraction of specific features from peaks within the waveform. The extraction of such features is often hindered by factors such as noise which in turn limits the efficacy of these features being used in downstream analysis. Moreover, building models to accurately represent the underlying peaks within the waveform are often limited by the amount of in vitro data on which such models can be trained.

Therefore, there is a need for new approaches to processing functional response data which allow for accurate and efficient feature extraction.

Moreover, existing approaches typically rely on the tissue exhibiting regular, or periodic, contractile responses (e.g., due to an external stimulation being applied to the tissue). Existing approaches may fail in adequately identifying individual contractile responses, and extracting relevant features, when the tissue exhibits spontaneous, or irregular, contractile behavior.

Therefore, there is a need for new approaches to detecting and processing spontaneous behavior in functional response data.

Further, many functional response waveforms may also comprise a combination of single and double contractions (e.g., ectopic beats). The ability to process such waveforms is often dependent on the ability to identify the contraction types present.

Therefore, there is a need for new approaches to processing functional response data which allow for accurate and efficient feature extraction and effective classification of contraction type.

Still further, in some systems, a tissue may be attached within a device to a tissue scaffold such that an image capturing the deflection of the tissue scaffold may be used to obtain measurements of tissue response at the time point the image was captured. However, the conversion of the position of a tissue scaffold within an image frame to a measure of contractile force may be inhibited by inconsistent or inaccurate tracking of the tissue scaffold or inaccurate conversion of the scaffold deflection to a contractile force.

Therefore, there is a need for new approaches to track the deflection of a tissue scaffold and subsequently model the force exerted on the tissue scaffold.

### SUMMARY OF DISCLOSURE

### Systems and Methods for Processing of Functional Response Waveforms.

According to an aspect of the present disclosure there is provided a method for processing a functional response waveform. The method comprises obtaining a first waveform comprising a contraction response and a relaxation response of an artificial tissue during a single contraction-relaxation cycle, fitting a model to the first waveform, wherein the model independently parameterizes growth of the contraction response and the relaxation response, and generating a second waveform from the model fit to the first waveform such that the second waveform comprises a noise filtered representation of the first waveform.

According to a further aspect of the present disclosure there is provided a method for training a model using synthetic training data. The method comprises obtaining a plurality of waveforms comprising functional responses of one or more artificial tissues during a single contraction-relaxation cycle, extracting a plurality of parameter sets from the plurality of waveforms, wherein a parameter set of the plurality of parameter sets characterizes a corresponding waveform of the plurality of waveforms, and determining a parameter set distribution from the plurality of parameter sets. The method further comprises generating a synthetic training data set, each element of the synthetic training data set comprising a synthetic waveform and a corresponding parameter set used to generate the synthetic waveform, wherein the corresponding parameter set is obtained from the parameter set distribution, and training a prediction model using the synthetic training data set, wherein the prediction model is trained to estimate an output parameter set from an input waveform.

According to an additional aspect of the present disclosure there is provided a method for extraction a contraction-relaxation cycle waveform. The method comprises obtaining a first waveform comprising a plurality of functional responses of an artificial tissue stimulated at a first frequency, and convolving, by the one or more processors, the first waveform with a pulse-train to generate a convolved waveform, wherein the pulse-train is generated at the first frequency. The method further comprises identifying a first location associated with a maximum value of the convolved waveform, wherein the first location corresponds to an expected location of a first contraction-relaxation cycle, and extracting, from the first location of the first waveform, a second waveform comprising the first contraction-relaxation cycle, wherein the second waveform has a first duration proportional to the first frequency.

According to a further aspect of the present disclosure there is provided a method for predicting a treatment effect. The method comprises obtaining a plurality of signals comprising a baseline signal and a perturbation signal, wherein the baseline signal comprises a first plurality of functional responses of an engineered tissue under reference conditions and the perturbation signal comprises a second plurality of functional responses of the engineered tissue under perturbed conditions involving a first perturbation. The method further comprises splitting the plurality of signals into a first plurality of waveforms, each waveform of the first plurality of waveforms comprising a contraction response and a relaxation response of the engineered tissue during a single contraction-relaxation cycle. The method further comprises fitting a model to each waveform of the first plurality of waveforms, wherein the model independently parameterizes growth of the contraction response and the relaxation response of the engineered tissue during the single contraction-relaxation cycle of each waveform, and generating a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal. The method further comprises extracting a first feature value of a first feature from the plurality of filtered baseline waveforms, extracting a second feature value of the first feature from the plurality of filtered perturbation waveforms, and determining an effect associated with the first perturbation based on a comparison of the first feature value and the second feature value.

In a still further aspect of the present disclosure there is provided a method for processing a functional response waveform. The method comprises obtaining, by one or more processors, a first waveform comprising a contraction response and a relaxation response of an artificial tissue during a single contraction-relaxation cycle, wherein the first waveform is obtained from a bioreactor comprising the artificial tissue. The method further comprises fitting, by the one or more processors, a model to the first waveform, wherein the model independently parameterizes growth of the contraction response and the relaxation response. The method further comprises generating, by the one or more processors, a second waveform from the model fit to the first waveform such that the second waveform comprises a noise filtered representation of the first waveform. The method further comprises extracting, by the one or more processors, one or more feature values from the second waveform. The method further comprises training a machine learning model on the one or more feature values from the second waveform. The method further comprises generating, by inputting a data set of a human tissue into the machine learning model, a prediction defining one or more characteristics of the human tissue, the prediction corresponding to at least one of the one or more features values of the second waveform.

In accordance with the above, and with the disclosure herein, the present disclosure includes applying certain features or aspects with or by use of, a particular machine, e.g., a bioreactor. In various aspects, the bioreactor may comprise a device configured for growing or manipulating human tissue (e.g., human body tissue such as muscle tissue, cardiac tissue, and/or skeletal muscle tissue). Additionally, or alternatively, bioreactor may comprise a sensor assembly configured to detect one or more functional responses of the tissue within the device.

Further, the present disclosure includes effecting a transformation or reduction of a particular article to a different state or thing, e.g., the transformation or reduction of functional responses of human tissue, e.g., within a bioreactor as sensed by a sensor assembly as waveforms, to a different state or thing, e.g., the generation, creation, or more accurate fitting of models based on improved waveforms that have been filtered to remove error (e.g., noise) in an originally received or raw waveform signal as sensed (e.g., by one or more sensors) from an artificial tissue.

Still further, the present disclosure includes improvements in computer functionality or in improvements to other technologies at least because the disclosure herein discloses systems and methods for reducing error in underlying computing devices, e.g., by producing enhanced (e.g., second) waveforms filtered from data noise caused by, e.g., a measurement hardware (e.g., a sensor assembly extracting data from the artificial tissue). Still further, once an enhanced (e.g., second) waveform is generated, fit, or otherwise obtained, high-fidelity training data sets may then be trained or generated therefrom. Such high-fidelity and high-volume synthetic training data sets may then be used to train or update models, such as new or updated machine learning models, in order to produce more accurate and less error prone output, and, as a result, high-quality drug products, such as therapeutics. Still further, the prediction models, when deployed on the underlying system, allows the systems and methods of the present disclosure to execute with fewer iterations, and use fewer computing resources, than prior art related systems and methods. That is, the present disclosure describes improvements in the functioning of the computer itself or “any other technology or technical field” because the increased predictive improvement provided by the prediction model allows the underlying computer system to utilize less processing and memory resources compared to prior art systems and methods. This at least because the prediction model, trained on a reduced error (less noisy) enhanced or second waveform, can generate or determine a probability that a given tissue will exhibit a given behavior with higher accuracy, without the need for various tests and/or empirical computer simulation across a wide range of tests using multiple compute cycles and data. Therefore use of the prediction model results in fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials on human tissue that can result in, and require, large database and memory utilization and processor usage to arrive at a similar real-world or simulated results that has a same or similar result. On the other hand, the disclosed systems and methods describe generation and/or use of a bioreactor for growing and testing tissue for defining a limited set of data specific to the tissue (e.g., human engineered tissue), which requires less memory usage and/or processing utilization compared to a conventional approach where large sets of unknown, potentially irrelevant data is used or required. Moreover, the disclosure herein allows for identification and use of high-fidelity data sets, which reduces the need for additional computational cycles further.

In addition, the present disclosure relates to improvement to other technologies or technical fields at least because the systems and methods of the present disclosure provide a robust, efficient, and comparable encoding of tissue behavior that can be used to improve the efficiency and performance of several downstream drug discovery and development tasks. This may be performed, for example, by a prediction model that is trained or otherwise generated with spectral representations as training data that defines functional responses of tissue (e.g., engineered human tissue). The prediction model may be deployed on an underlying computing device or system, thereby, improving its accuracy and prediction in performing drug discovery and development tasks as described herein.

Still further, the present disclosure includes specific features other than what is well-understood, routine, conventional activity in the field, and/or otherwise adds unconventional steps that confine the disclosure to a particular useful application, e.g., systems and methods for processing functional response waveforms of artificial tissue for the purpose of generating high-fidelity feature data, which can be used, for example, to train more accurate models and/or improve downstream tasks such as drug discovery and development.

### Systems and Methods for Detecting Spontaneous Tissue Contractions.

According to an aspect of the present disclosure there is provided a method for determining the spontaneous behavior of engineered tissue. The method comprises obtaining a waveform comprising a functional response of an engineered tissue over a time period and applying a frequency-based global classifier to the waveform thereby generating a first classification score, wherein the first classification score is indicative of whether the waveform comprises periodic contractions of the engineered tissue over the time period. When the first classification score is indicative of no periodic contractions being present within the waveform, the method further comprises applying a local classifier to the waveform thereby generating a second classification score, wherein the second classification score is indicative of whether the waveform comprises spontaneous contractions of the engineered tissue over the time period, and generating a behavior profile for the engineered tissue during the time period based on the second classification score.

According to a further aspect of the present disclosure there is provided a system for determining the spontaneous behavior of engineered tissue. The system comprises a bioreactor comprising a device configured for growing tissue, and a sensor assembly configured to detect one or more functional responses of an engineered tissue within the device. The system further comprises a processing unit communicatively coupled to the bioreactor, wherein the processing unit comprises one or more processors configured to obtain a waveform comprising a functional response of the engineered tissue within the device over a time period, and apply a periodicity classifier to the waveform thereby generating a first classification score, wherein the first classification score is indicative of whether the waveform comprises periodic contractions of the engineered tissue over the time period. When the first classification score is indicative of no periodic contractions being present within the waveform, the one or more processors are configured to apply a spontaneous contraction classifier to the waveform thereby generating a second classification score, wherein the second classification score is indicative of whether the waveform comprises spontaneous contractions of the engineered tissue over the time period, and generate a behavior profile for the engineered tissue during the time period based on the second classification score.

According to an additional aspect of the present disclosure there is provided a non-transitory computer-readable medium storing instructions for determining the spontaneous behavior of engineered tissue. The instructions which, when executed by one or more processors, cause the one or more processors to obtain a waveform comprising a functional response of an engineered tissue over a time period, and apply a first classifier to the waveform thereby generating a first classification score, wherein the first classification score is indicative of whether the waveform comprises periodic contractions of the engineered tissue over the time period. When the first classification score is indicative of no periodic contractions being present within the waveform the one or more processors are further caused to apply a second classifier to the waveform thereby generating a second classification score, wherein the second classification score is indicative of whether the waveform comprises spontaneous contractions of the engineered tissue over the time period, and generate a behavior profile for the engineered tissue during the time period based on the second classification score.

In accordance with the above, and with the disclosure herein, the present disclosure includes applying certain features or aspects with or by use of, a particular machine, e.g., a bioreactor. In various aspects, the bioreactor may comprise a device configured for growing or manipulating human tissue (e.g., human body tissue such as muscle tissue, cardiac tissue, and/or skeletal muscle tissue). Additionally, or alternatively, bioreactor may comprise a sensor assembly configured to detect one or more functional responses of the tissue within the device.

Further, the present disclosure includes effecting a transformation or reduction of a particular article to a different state or thing, e.g., the transformation or reduction of functional responses of human tissue, e.g., within a bioreactor as sensed by a sensor assembly as waveforms, to a different state or thing, e.g., the generation, creation, or otherwise development of a behavior profile for the engineered tissue based on an originally received or raw waveform signal as sensed (e.g., by one or more sensors) from an artificial tissue.

Still further, the present disclosure includes improvements in computer functionality or in improvements to other technologies at least because the disclosure herein discloses systems and methods for reducing error in underlying computing devices, e.g., by implement a hierarchical system of multiple classifiers, where one classifier (e.g., a periodicity classifier) may filter out waveforms which are known to exhibit periodic, or regular, behavior. Thereby, the amount data used by the system is decreased, where only waveforms which are predicted not to have periodic, or regular, behavior are fed to later local classifier (e.g., a spontaneous classifier). This not only reduces the amount of data analyzed by the underlying computing system, but also streamlines ingestion of the system to waveforms (or waveform data) having the potential to exhibit spontaneous behavior thereby providing improved identification of the spontaneous behavior (and the locations of spontaneous contractions) because the system would have fewer false positives as it would only be analyzing a narrow, or otherwise reduced, set of suspect data. Said another way, once waveforms (or waveform data) relating to spontaneous behavior of an engineered tissue is identified by a classifier, then then such data can be selected (filtered) for analysis. Such waveform data comprises high-fidelity data for generating a behavior profile for the engineered tissue, in order to produce more accurate and less error prone output, and, as a result, high-quality drug products, such as therapeutics.

Still further, the hierarchy of classifiers, when deployed on the underlying system, allows the systems and methods of the present disclosure to execute with fewer iterations, and use fewer computing resources, than prior art related systems and methods. That is, the present disclosure describes improvements in the functioning of the computer itself or “any other technology or technical field” because the increased predictive improvement provided by the hierarchy of classifiers allows the underlying computer system to utilize less processing and memory resources compared to prior art systems and methods. This at least because the hierarchy of classifiers, designed to filter out periodic waveform data to provide reduced error (less noisy) spontaneous waveform data, can generate or determine a probability that a given tissue will exhibit a given behavior with higher accuracy, without the need for various tests and/or empirical computer simulation across a wide range of tests using multiple compute cycles and data. Therefore use of the hierarchy of classifiers results in fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials on human tissue that can result in, and require, large database and memory utilization and processor usage to arrive at a similar real-world or simulated results that has a same or similar result. On the other hand, the disclosed systems and methods describe generation and/or use of a bioreactor for growing and testing tissue for defining a limited set of data specific to the tissue (e.g., human engineered tissue), which requires less memory usage and/or processing utilization compared to a conventional approach where large sets of unknown, potentially irrelevant data is used or required. Moreover, the disclosure herein allows for identification and use of high-fidelity data sets, which reduces the need for additional computational cycles further.

In addition, the present disclosure relates to improvement to other technologies or technical fields at least because the systems and methods of the present disclosure provide a robust, efficient, and comparable encoding of tissue behavior that can be used to improve the efficiency and performance of several downstream drug discovery and development tasks. This may be performed, for example, by a hierarchy of classifiers that are employed to filter, via classification, specific waveform type data (e.g., spontaneous waveforms and/or related data), which can then be used to identify or define functional responses of tissue (e.g., engineered human tissue). The hierarchy of classifiers may be deployed on an underlying computing device or system, thereby, improving its accuracy, classification, and/or prediction in performing drug discovery and development tasks as described herein.

Still further, the present disclosure includes specific features other than what is well-understood, routine, conventional activity in the field, and/or otherwise adds unconventional steps that confine the disclosure to a particular useful application, e.g., systems and methods for determining the spontaneous behavior of engineered tissue, which can be used, for example, to determine more accurate behavior profile(s) based on engineered tissue, which can improve downstream tasks such as drug discovery and development.

### Systems and Methods for Contraction Type Classification of Engineered Tissue.

According to an aspect of the present disclosure there is provided a method for processing a functional response waveform. The method comprises obtaining a first waveform comprising at least one contraction response and at least one relaxation response of an engineered tissue, the first waveform having a predetermined length corresponding to an expected length of a contraction-relaxation cycle of the engineered tissue. The method further comprises determining a predicted contraction type of a plurality of contraction types for the first waveform, wherein the plurality of contraction types include a single contraction type and a double contraction type. The method further comprises fitting a model to the first waveform based on the predicted contraction type, wherein the model parameterizes growth of the at least one contraction response of the engineered tissue independently of growth of the at least one relaxation response of the engineered tissue.

The method further comprises generating a second waveform from the model fit to the first waveform such that the second waveform comprises a noise filtered representation of the first waveform.

According to a further aspect of the present disclosure there is provided a method for training a classifier to predict a tissue contraction type from a functional response waveform. The method comprises obtaining a plurality of waveforms comprising functional responses of a one or more engineered tissues, each of the plurality of waveforms having a predetermined length corresponding to an expected length of a contraction-relaxation cycle of an engineered tissue. The method further comprises extracting, by the one or more processors, a first plurality of parameter sets from a first subset of the plurality of waveforms associated with a single contraction type, wherein a first parameter set of the first plurality of parameter sets characterizes a first waveform of the first subset of waveforms. The method further comprises extracting a second plurality of parameter sets from a second subset of the plurality of waveforms associated with a double contraction type, wherein a second parameter set of the second plurality of parameter sets characterizes a second waveform of the second subset of waveforms. The method further comprises determining a plurality of parameter set distributions, wherein the plurality of parameter set distributions comprise a first parameter set distribution determined from the first plurality of parameter sets and a second parameter set distribution determined from the second plurality of parameter sets. The method further comprises generating a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type. The method further comprises training the classifier using the synthetic training data set, wherein the classifier trained using the synthetic training data set determines a predicted tissue contraction type for an input waveform.

According to an additional aspect of the present disclosure there is provided a method for processing a functional response waveform. The method comprises obtaining a first waveform comprising a plurality of functional responses of an artificial tissue stimulated at a first frequency, and convolving the first waveform with a pulse-train to generate a convolved waveform, wherein the pulse-train is generated at the first frequency. The method further comprises identifying a first location associated with a first maximum value of the convolved waveform, wherein the first location corresponds to an expected location of a first contraction-relaxation cycle. The method further comprises extracting, from the first location of the first waveform, a second waveform comprising the first contraction-relaxation cycle, wherein the second waveform has a first duration proportional to the first frequency.

According to a further aspect of the present disclosure there is provided a method for predicting a perturbation effect. The method comprises obtaining a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions. The method further comprises splitting the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue. The method further comprises determining a predicted contraction type of a plurality of contraction types for each of the first plurality of waveforms, wherein the plurality of contraction types include a single contraction type and a double contraction type. The method further comprises fitting a model to each waveform of the first plurality of waveforms based on a corresponding predicted contraction type, wherein the model parameterizes growth of the at least one contraction response independently of growth of the at least one relaxation response of the engineered tissue. The method further comprises generating a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal. The method further comprises extracting a first feature value of a first feature from the plurality of filtered baseline waveforms, extracting a second feature value of the first feature from the plurality of filtered treatment waveforms, and determining an effect associated with the first set of perturbation conditions based on a comparison of the first feature value and the second feature value.

According to an additional aspect of the present disclosure there is provided a method for training a model using synthetic training data. The method comprises obtaining a plurality of waveforms comprising functional responses of one or more artificial tissues during a single contraction-relaxation cycle and extracting a plurality of parameter sets from the plurality of waveforms, wherein a parameter set of the plurality of parameter sets characterizes a corresponding waveform of the plurality of waveforms. The method further comprises determining a parameter set distribution from the plurality of parameter sets and generating a synthetic training data set, each element of the synthetic training data set comprising a synthetic waveform and a corresponding parameter set used to generate the synthetic waveform, wherein the corresponding parameter set is obtained from the parameter set distribution. The method further comprises training a parameter estimation model using the synthetic training data set, wherein the parameter estimation model is trained to estimate an output parameter set from an input waveform.

In accordance with the above, and with the disclosure herein, the present disclosure includes applying certain features or aspects with or by use of, a particular machine, e.g., a bioreactor. In various aspects, the bioreactor may comprise a device configured for growing or manipulating human tissue (e.g., human body tissue such as muscle tissue, cardiac tissue, and/or skeletal muscle tissue). Additionally, or alternatively, bioreactor may comprise a sensor assembly configured to detect one or more functional responses of the tissue within the device.

Further, the present disclosure includes effecting a transformation or reduction of a particular article to a different state or thing, e.g., the transformation or reduction of functional responses of human tissue, e.g., within a bioreactor as sensed by a sensor assembly as waveforms, to a different state or thing, e.g., the generation, creation, or otherwise development of noise-filtered second waveform using a fit model and based on an originally received or raw waveform signal as sensed (e.g., by one or more sensors) from an artificial tissue.

Still further, the present disclosure includes improvements in computer functionality or in improvements to other technologies at least because the disclosure herein discloses systems and methods for reducing error in underlying computing devices, e.g., by implementing a contraction-relaxation cycle model where a first waveform of an engineered tissue is improved by noise filtering using model fitting to eliminate or reduce data usage and also to enable high-fidelity and high-volume synthetic training data sets to be generated. The contraction-relaxation cycle model is also efficiently extendible to model different contraction types (e.g., a functional response waveform comprising a single or double contractile response). Thereby, the amount data used by the system is decreased, where waveforms that have been noise reduced (and thus data reduced) may be used as output for end processes, such as for output for, e.g., improved downstream tasks such as drug discovery and development. This not only reduces the amount of data analyzed by the underlying computing system, but also streamlines ingestion of the system to waveforms (or waveform data) having both a contraction response and a relaxation response because the system would have fewer false positives as it would only be analyzing a filtered, or otherwise reduced, set of data that is high-fidelity as it would rely on synthetic (controlled) data as determined by the model. Such data is also extendable because it allows for the model predict, classify, or otherwise output different contraction types (e.g., a functional response waveform comprising a single or double contractile response). Said another way, once waveforms (or waveform data) relating to a relaxation response and a contraction response of an engineered tissue are input, such data can be input to a model (e.g., classifier model) for determining or otherwise classifying a plurality of contraction types (e.g., a single contraction type and/or a double contraction type) identified by the model, then such data can be selected (filtered) for analysis. Such waveform data comprises high-fidelity data for fitting an input waveform (e.g., a first waveform) to the model to parameterize growth of contraction response of the engineered tissue independent of growth of the relaxation response of the engineered tissue for generating a second noise filtered version of the input (first) waveform. This also allows for generation of more accurate and less error prone output, and, as a result, high-quality drug products, such as therapeutics.

Still further, the contraction-relaxation cycle model, when deployed on the underlying system, allows the systems and methods of the present disclosure to execute with fewer iterations, and use fewer computing resources, than prior art related systems and methods. That is, the present disclosure describes improvements in the functioning of the computer itself or “any other technology or technical field” because the increased predictive improvement provided by the contraction-relaxation cycle model allows the underlying computer system to utilize less processing and memory resources compared to prior art systems and methods. This at least because the contraction-relaxation cycle model, designed to filter out periodic waveform data to provide reduced error (less noisy) waveform data, can generate or determine a probability that a given tissue will exhibit a given behavior with higher accuracy, without the need for various tests and/or empirical computer simulation across a wide range of tests using multiple compute cycles and data. Therefore use of the hierarchy of classifiers results in fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials on human tissue that can result in, and require, large database and memory utilization and processor usage to arrive at a similar real-world or simulated results that has a same or similar result. On the other hand, the disclosed systems and methods describe generation and/or use of a bioreactor for growing and testing tissue for defining a limited set of data specific to the tissue (e.g., human engineered tissue), which requires less memory usage and/or processing utilization compared to a conventional approach where large sets of unknown, potentially irrelevant data is used or required. Moreover, the disclosure herein allows for identification and use of high-fidelity data sets, which reduces the need for additional computational cycles further.

In addition, the present disclosure relates to improvement to other technologies or technical fields at least because the systems and methods of the present disclosure provide a robust, efficient, and comparable encoding of tissue behavior that can be used to improve the efficiency and performance of several downstream drug discovery and development tasks. This may be performed, for example, by a contraction-relaxation cycle model (e.g., a type of classifier) that are employed to filter, via classification, specific waveform type data (e.g., waveforms and/or related data), which can then be used to identify, classify, or define contraction types (single and/or double contraction types) of engineered human tissue. The contraction-relaxation cycle model may be deployed on an underlying computing device or system, thereby, improving its accuracy, classification, and/or prediction in performing drug discovery and development tasks as described herein.

Still further, the present disclosure includes specific features other than what is well-understood, routine, conventional activity in the field, and/or otherwise adds unconventional steps that confine the disclosure to a particular useful application, e.g., systems and methods for contraction type classification of engineered tissue, which can be used, for example, to determine noise filtered and/or model fitted waveform(s) based on engineered tissue, which can improve downstream tasks such as drug discovery and development.

### Systems and Methods for Tissue Scaffold Tracking.

According to an aspect of the present disclosure there is provided a system for modelling contractile deflection of flexible tissue scaffolds. The system comprises a bioreactor comprising a flexible scaffold for attachment to biological tissue, wherein the flexible scaffold is arranged to deflect in response to contractile force exerted thereupon. The bioreactor further comprises an imaging apparatus configured to obtain one or more images of the flexible scaffold. The system further comprises a processing unit communicatively coupled to the bioreactor. The processing unit is configured to obtain, from the bioreactor, a plurality of images of the flexible scaffold at a plurality of time points, wherein the plurality of images capture deflections of the flexible scaffold along a first dimension due to contractile forces exerted thereupon at the plurality of time points. The processing unit is further configured to fit a plurality of curves to the plurality of images such that each curve extends along a centerline of the flexible scaffold within a respective image of the plurality of images and determine a plurality of displacement values from the plurality of curves, wherein each of the plurality of displacement values comprise a measurement between a respective curve of the plurality of curves and a reference line extending along a second dimension perpendicular to the first dimension. The processing unit is further configured to generate a model based on the plurality of displacement values, wherein the model characterizes contractile forces exerted on the flexible scaffold over the plurality of time points.

According to a further aspect of the present disclosure there is provided a method for modelling contractile deflection of flexible tissue scaffolds. The method comprises obtaining a plurality of images of a flexible scaffold at a plurality of time points, wherein the plurality of images capture deflections of the flexible scaffold along a first dimension due to contractile forces exerted thereupon at the plurality of time points. The method further comprises fitting a plurality of curves to the plurality of images such that each curve extends along a centerline of the flexible scaffold within a respective image of the plurality of images and determining a plurality of displacement values from the plurality of curves, wherein each of the plurality of displacement values comprises a measurement along the first dimension between a respective curve of the plurality of curves and a reference line extending along a second dimension perpendicular to the first dimension. The method further comprises generating a model based on the plurality of displacement values, wherein the model characterizes contractile forces exerted on the flexible scaffold over the plurality of time points.

According to an additional aspect of the present disclosure there is provided a non-transitory computer-readable medium storing instructions which, when executed by a processing unit, cause the processing unit to obtain a plurality of images of a flexible scaffold at a plurality of time points, wherein the plurality of images capture deflections of the flexible scaffold along a first dimension due to contractile forces exerted thereupon at the plurality of time points; fit a plurality of curves to the plurality of images such that each curve extends along a centerline of the flexible scaffold within a respective image of the plurality of images; determine a plurality of displacement values from the plurality of curves, wherein each of the plurality of displacement values comprises a measurement along the first dimension between a respective curve of the plurality of curves and a reference line extending along a second dimension perpendicular to the first dimension; and generate a model based on the plurality of displacement values, wherein the model characterizes contractile forces exerted on the flexible scaffold over the plurality of time points.

In accordance with the above, and with the disclosure herein, the present disclosure includes applying certain features or aspects with or by use of, a particular machine, e.g., a bioreactor. In various aspects, the bioreactor may comprise a device configured for growing or manipulating human tissue (e.g., human body tissue such as muscle tissue, cardiac tissue, and/or skeletal muscle tissue). Additionally, or alternatively, bioreactor may comprise a sensor assembly configured to detect one or more functional responses of the tissue within the device.

Further, the present disclosure includes effecting a transformation or reduction of a particular article to a different state or thing, e.g., the transformation or reduction of displacement values of a flexible scaffold, e.g., within a bioreactor as sensed by an imaging assembly, to a different state or thing, e.g., the generation, creation, or otherwise determination of a contractile force of an engineered tissued attached to flexible scaffold tissue.

Still further, the present disclosure includes improvements in computer functionality or in improvements to other technologies at least because the disclosure herein discloses systems and methods for reducing error in underlying computing devices, e.g., by using a flexible scaffold to accurately track and efficiently extract and measure deflections of tissue scaffolds thereby allowing a model to be generated that characterizes the contractile force(s) which produced the deflections. The underlying system can be updated with the model, where the model can be used to encode or calibrate the relationship between contractile force and measured displacement thereby improving the accuracy of contractile force measurements obtained from such models. Improvements to such models provide improvements to the downstream tasks which utilize such models whilst also improving the efficiency and performance of computing systems, which are used to generate and deploy such models. This also allows for generation of more accurate and less error prone output, and, as a result, high-quality drug products, such as therapeutics.

Still further, the contraction-relaxation cycle model, when deployed on the underlying system, allows the systems and methods of the present disclosure to execute with fewer iterations, and use fewer computing resources, than prior art related systems and methods. That is, the present disclosure describes improvements in the functioning of the computer itself or “any other technology or technical field” because the increased predictive improvement provided by implementing a model (e.g., a force-displacement model) generated for a specific device and/or tissue scaffold thereby allowing any variances in contractile responses of the tissue scaffold to be modelled within the force-displacement model thereby improving the consistency of force measurements obtained from displacement values provided to the model. Therefore use of the model (e.g., a force-displacement model) results in fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials on human tissue that can result in, and require, large database and memory utilization and processor usage to arrive at a similar real-world or simulated results that has a same or similar result. On the other hand, the disclosed systems and methods describe generation and/or use of a bioreactor for growing and testing tissue for defining a limited set of data specific to the tissue (e.g., human engineered tissue), which requires less memory usage and/or processing utilization compared to a conventional approach where large sets of unknown, potentially irrelevant data is used or required. Moreover, the disclosure herein allows for identification and use of high-fidelity data sets, which reduces the need for additional computational cycles further.

In addition, the present disclosure relates to improvement to other technologies or technical fields at least because the systems and methods of the present disclosure provide a robust, efficient, and comparable tissue measurement solution that can be used to improve the efficiency and performance of several downstream drug discovery and development tasks. This may be performed, for example, by a flexible scaffold and imaging apparatus configured to generate a model based on displacement values such that the model can characterize contractile forces exerted on the flexible scaffold. The model can then be used to identify, classify, or define tissue, such as engineered human tissue. The model may be deployed on an underlying computing device or system, thereby, improving its accuracy, classification, and/or prediction in performing drug discovery and development tasks as described herein.

Still further, the present disclosure includes specific features other than what is well-understood, routine, conventional activity in the field, and/or otherwise adds unconventional steps that confine the disclosure to a particular useful application, e.g., systems and methods for modelling contractile deflection of flexible tissue scaffolds and for generating a model characterizing contractile forces exerted on a flexible tissue scaffold over a plurality of time points, which can improve downstream tasks such as drug discovery and development.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF DRAWINGS

Embodiments of the present disclosure will now be described, by way of example only, and with reference to the accompanying drawings, in which:

FIG. 1 shows a system for processing functional response data according to an aspect of the present disclosure;

FIG. 2 shows a waveform comprising a functional response of an engineered tissue obtained from the system of FIG. 1 according to an aspect of the present disclosure;

FIG. 3 illustrates a contraction-relaxation cycle model according to an embodiment of the present disclosure;

FIG. 4 shows example waveforms with corresponding contraction-relaxation cycle model fits according to an aspect of the present disclosure;

FIGS. 5A and 5B illustrate the effect of each parameter of a contraction-relaxation cycle model according to an embodiment of the present disclosure;

FIG. 6 shows a parameter estimation model for fitting the parameters of a contraction-relaxation-cycle model according to an embodiment of the present disclosure;

FIGS. 7A-7D illustrate elements of the parameter estimation model shown in FIG. 6 according to an embodiment of the present disclosure;

FIG. 8 shows a method for processing a functional response waveform according to an aspect of the present disclosure;

FIG. 9 shows a method for fitting a model to a functional response waveform according to an embodiment of the present disclosure;

FIG. 10 shows a method for training a parameter estimation model using synthetic training data according to an aspect of the present disclosure;

FIG. 11 shows a method for predicting a set of parameter values using the parameter estimated model training according to the method of FIG. 10 according to an embodiment of the present disclosure;

FIG. 12 shows a method for extracting a contraction-relaxation cycle waveform according to an aspect of the present disclosure;

FIG. 13 shows a method for extracting a further contraction-relaxation cycle waveform according to an embodiment of the present disclosure;

FIG. 14 shows a method for predicting a treatment effect using a contraction-relaxation cycle model according to an aspect of the present disclosure;

FIG. 15A shows the functional response of a tissue exhibiting periodic contractile behavior according to an aspect of the present disclosure;

FIG. 15B shows the functional response of a tissue exhibiting spontaneous contractile behavior according to an aspect of the present disclosure;

FIG. 16 shows a system for determining the spontaneous behavior of engineered tissue according to an aspect of the present disclosure;

FIGS. 17A and 17B show spectral responses of different contractile responses according to embodiments of the present disclosure;

FIG. 18 shows a convolutional neural network for predicting contractile behavior according to an embodiment of the present disclosure;

FIG. 19 illustrates the step-wise results of performing a modified Pan-Tompkins algorithm according to an embodiment of the present disclosure;

FIG. 20 illustrates adaptive thresholding of a waveform according to an embodiment of the present disclosure;

FIG. 21 shows a method for determining the spontaneous behavior of engineered tissue according to an aspect of the present disclosure;

FIG. 22 shows a method for obtaining a classification score from a waveform according to an embodiment of the present disclosure;

FIG. 23 shows a method for obtaining a classification score from a waveform according to an embodiment of the present disclosure;

FIG. 24 shows a modified Pans-Tompkins method according to an embodiment of the present disclosure;

FIG. 25 illustrates a double contraction type contraction-relaxation cycle model according to an embodiment of the present disclosure;

FIG. 26 shows a system for processing of functional response waveforms having different contraction types according to an embodiment of the present disclosure;

FIG. 27 shows contraction type classifications for three waveforms according to an embodiment of the present disclosure;

FIG. 28 shows a method for processing a functional response waveform according to an aspect of the present disclosure;

FIG. 29 shows a method for fitting a model to a functional response waveform according to an embodiment of the present disclosure;

FIG. 30 shows a method for training a classifier to predict a tissue contraction type from a functional response waveform;

FIG. 31 shows a method for predicting a tissue contraction type for a waveform using a synthetically trained classifier according to an embodiment of the present disclosure;

FIG. 32 shows a method for training a parameter estimation model using synthetic training data according to an aspect of the present disclosure;

FIG. 33 shows a method for extracting a single or double contraction-relaxation cycle waveform according to an aspect of the present disclosure;

FIG. 34 shows a method for extracting a further single or double contraction-relaxation cycle from a waveform according to an embodiment of the present disclosure;

FIG. 35 shows a method for predicting a perturbation effect according to an aspect of the present disclosure;

FIG. 36 shows a well of a bioreactor, such as the bioreactor shown in FIG. 1, according to an embodiment of the present disclosure;

FIG. 37 shows example images of tissue scaffolds under different contractile forces according to embodiments of the present disclosure;

FIG. 38 shows an approach for flexible scaffold tracking according to an aspect of the present disclosure;

FIG. 39 shows a one-dimensional vessel enhancement filter according to an embodiment of the present disclosure;

FIG. 40 shows a plot of data points obtained from a transformed image of a tissue scaffold according to an embodiment of the present disclosure;

FIG. 41 shows a generated model corresponding to a time-series of scaffold deflection values according to an embodiment of the present disclosure;

FIG. 42 shows a flexible scaffold deflecting due to a predetermined force exerted thereupon by a probe according to an embodiment of the present disclosure;

FIG. 43 shows a plot of force-displacement models according to embodiments of the present disclosure;

FIG. 44 shows a method for modelling contractile deflection values of flexible tissue scaffolds according to an aspect of the present disclosure;

FIG. 45 shows a method for fitting a plurality of curves according to an embodiment of the present disclosure;

FIG. 46 shows a method for fitting a reference line according to an embodiment of the present disclosure; and

FIG. 47 shows an example computing system for carrying out the methods of the present disclosure according to one embodiment.

### TECHNICAL FIELD

The present disclosure relates to processing functional response waveforms. Particularly, but not exclusively, the present disclosure relates to processing functional response waveforms using a contraction-relaxation cycle model. More particularly, but not exclusively, the present disclosure relates to generating noise filtered representations of functional response waveforms using the contraction-relaxation cycle model. More particularly again, but not exclusively, the present disclosure relates to identifying effects associated with perturbations of an engineered tissue based on noise filtered functional response waveforms generated using the contraction-relaxation cycle model.

In addition, the present disclosure relates to detecting tissue behavior. Particularly, but not exclusively, the present disclosure relates to detecting spontaneous tissue contractions. Particularly, but not exclusively, the present disclosure relates to detecting spontaneous tissue contractions in functional response waveforms of engineered tissue.

Further, the present disclosure relates to classifying and processing functional response waveforms. Particularly, but not exclusively, the present disclosure relates to classifying and processing functional response waveforms using contraction-relaxation cycle models. More particularly, but not exclusively, the present disclosure relates to generating noise filtered representations of functional response waveforms using contraction-relaxation cycle models based on an identified contraction type. More particularly again, but not exclusively, the present disclosure relates to identifying effects associated with perturbations of an engineered tissue based on noise filtered functional response waveforms generated using the contraction-relaxation cycle models.

Still further, the present disclosure relates to tissue scaffold modelling. Particularly, but not exclusively, the present disclosure relates to modelling contractile deflection of flexible tissue scaffolds. Particularly, but not exclusively, the present disclosure relates to generating a model characterizing contractile forces exerted on a flexible tissue scaffold over a plurality of time points.

### DETAILED DESCRIPTION

The ability to extract features accurately and efficiently from a waveform of an engineered tissue's functional response is an important step when using such waveforms for downstream tasks such as drug discovery and development. Existing approaches are often limited by the quality and/or quantity of the available data. Moreover, the functional response of the engineered tissue may exhibit different response types making it difficult to fit a single model to all types of response. The present disclosure presents systems and methods regarding peak exploration and artificial tissue response to overcome such issues.

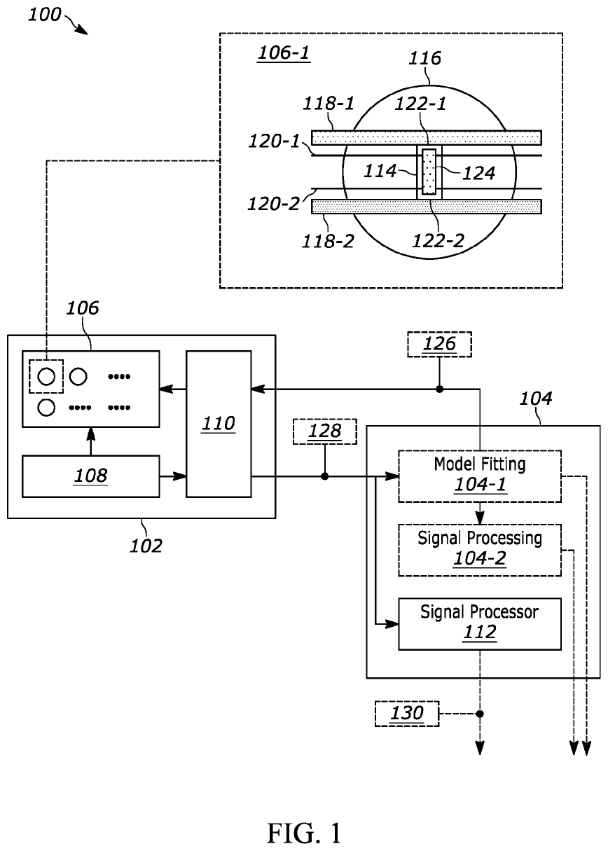

FIG. 1 shows a system 100 for processing functional response data or spontaneous tissue contraction data according to various aspects of the present disclosure. Additionally, or alternatively, system 100 also illustrates modelling contractile deflection of flexible tissue scaffolds according to an aspect of the present disclosure.

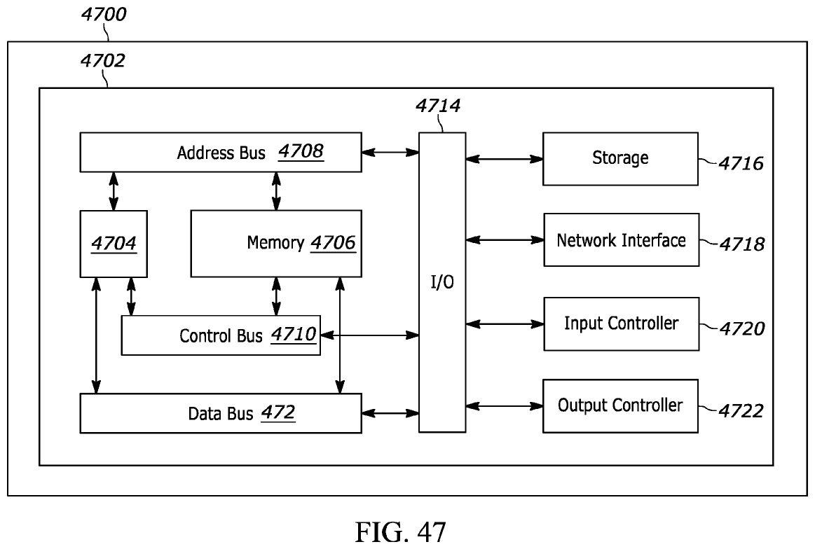

The system 100 comprises a bioreactor 102 and a control unit 104. The bioreactor 102 comprises a device 106 for growing engineered tissues, a sensor assembly 108, and an interface 110. In one embodiment, the sensor assembly 108 forms a part of the device 106, but such elements may be separate in other embodiments. The control unit 104 may comprise a model fitting unit 104-1, a signal processing unit 104-2, and a signal processing unit 112 (which may also be referred to as a signal processor). Additionally, or alternatively, the control unit 104 comprises signal processing unit 112 (alternatively referred to as a processing unit, signal processor, or processor). The processing unit may comprise one or more processor(s), for example one or more processor(s) as described herein for FIG. 47. The interface 110 communicatively couples the bioreactor 102 and the control unit 104 such that data may be exchanged between the bioreactor 102 and the control unit 104. In various aspects, the bioreactor is used for growing tissue (e.g., engineered tissue 124), such as human body tissue including, by way of non-limiting example, muscle tissue, cardiac tissue, skeletal muscle tissue, or other human tissue).

As shown in the expanded portion 106-1 of the device 106, the device 106, or substrate, comprises one or more wells, such as the well 114, one or more cell culture wells, such as the cell culture well 116, a pair of electrodes including a first electrode 118-1 and a second electrode 118-2, and a pair of elements (e.g., scaffolds) including a first element 120-1 (e.g. a first scaffold) and a second element 120-2 (e.g., a second scaffold). The well 114 is positioned within the cell culture well 116 and has a bottom on the device 106, a first end 122-1, and a second end 122-2. The well 114 is configured for growing an engineered tissue 124 from cells seeded therein. Culture medium may be added to the cell culture well 116 for growing and/or sustaining the engineered tissue 124. The engineered tissue 124, alternatively referred to as artificial tissue, comprises engineered muscle tissue. In one embodiment, the engineered muscle tissue is engineered cardiac tissue. For example, the engineered cardiac tissue is generated using Cellular Dynamics International (CDI) iCell Cardiomyocytes² and a side population of normal human ventricular cardiac fibroblasts embedded in a hydrogel composed of fibrin (Sigma-Aldrich), collagen (Sigma-Aldrich) and Matrigel (Corning). In an alternative embodiment, any other human cardiomyocytes are used in place of the iCell Cardiomyocytes², such as Axol Bioscience Human iPSC-Derived Ventricular Cardiomyocytes or Sigma-Aldrich Human Cardiac Myocytes (HCM). Additionally, or alternatively, fibrin and/or collagen are omitted from the hydrogel. In an alternative embodiment, the engineered muscle tissue is engineered skeletal muscle tissue.

The pair of electrodes are separated by a gap within which the well 114 is positioned. The pair of electrodes are configured to apply an electrical stimulation to cell cultures within the one or more wells of the device 106 (e.g., the engineered tissue 124 within the well 114 shown in the expanded portion 106-1). During maturation of the cell cultures within the device 106, the pair of electrodes apply stimulation to the cell cultures according to a multi-week electrical stimulation protocol. After the cell cultures are matured, the pair of electrodes may be configured to stimulate the cell cultures (e.g., the engineered tissue 124) at a set frequency, or pacing frequency. In one embodiment, the frequency, or pacing frequency, at which the cell cultures are stimulated is set by the control unit 104. As such, the control unit 104 may be configured to send an instruction 126 to the bioreactor 102 to cause the bioreactor 102 to stimulate the engineered tissue(s) within the device 106 at a set pacing frequency.

In some example embodiments, the first element 120-1 and the second element 120-2 are disposed across the well 114 such that there is a gap between the bottom of the well 114 and the pair of elements. The first element 120-1 and the second element 120-2 are configured to: (a) permit attachment of the engineered tissue 124 formed therebetween, thereby suspending the engineered tissue 124 above the bottom of the well 114, and (b) deform in response to the contractile force exerted on the pair of elements by the engineered tissue 124, thereby simulating a physiological environment that is native to the engineered tissue 124 and/or permitting measurement of the contractile force (e.g., by the sensor assembly 108). For example, the pair of electrodes may subject the engineered tissue 124 to an electrical stimulation at a frequency of 0.1 Hz. The engineered tissue 124 will contract in response to this electrical stimulation causing deformation of at least one of the first element 120-1 and the second element 120-2. Measuring the deformation of one or more pair(s) of elements allows the functional response of the engineered tissue 124 when stimulated at 0.1 Hz to be recorded.

The sensor assembly 108 is configured to detect one or more functional responses of an engineered tissue within the device 106 (e.g., one or more functional responses of the engineered tissue 124). In one embodiment, the sensor assembly 108 comprises an optical sensor. The optical sensor is configured to detect a deformation of the first element 120-1 and/or the second element 120-2 (e.g., occurring as a result of contractile force exerted on the first element 120-1 and/or the second element 120-2 by the engineered tissue 124). Detecting the deformation of the first element 120-1 and/or the second element 120-2 allows one or more functional responses such as a displacement, or contractile displacement, of the engineered tissue 124 or a contractile force of the engineered tissue 124 to be determined. Additionally, or alternatively, the optical sensor of the sensor assembly 108 is configured to detect a fluorescence intensity of the engineered tissue 124. Detecting the fluorescence intensity of the engineered tissue 124 allows one or more functional responses such as a transient calcium response of the engineered tissue 124 or a change in membrane potential of the engineered tissue 124 to be determined. Additionally, or alternatively, the optical sensor of the sensor assembly 108 is configured to detect a change in dimensions of the engineered tissue 124 over a time frame. Detecting the change in dimensions of the engineered tissue 124 over the time frame allows one or more functional responses such as the displacement, or contractile displacement, of the engineered tissue 124 or the contractile force of the engineered tissue 124 to be determined.

The optical sensor of the sensor assembly 108 is configured to obtain a plurality of image-based representations of the one or more functional responses of a tissue (e.g., the engineered tissue 124) over a time frame or time period. For example, the optical sensor may be configured to capture an image, or frame, of a tissue every n seconds. Here, n is associated with a predetermined rate at which the images or frames are to be captured. For example, when n=1 then one image of the tissue is captured per second. Any suitable value of n may be chosen such as n={1/60, 1/50, 1/30, 1/24, 1/12, 1/4, 1/2, 1,2} and the like. In one embodiment, the frame rate is determined according to the frequency at which the tissues within the device are being stimulated. The sequences of images or frames of a tissue over a time frame therefore captures one or more functional responses of the tissue over the time frame.

In one embodiment, the bioreactor 102 is configured to transform the sequence of images which capture the one or more functional responses of the tissue over the time frame to a waveform representation. For example, the sensor assembly 108, the interface 110, or another component of the bioreactor 102 may process an image within the sequence of images to extract features relating to the functional response of the tissue at a time point associated with the image. Features relating to the functional response of the tissue extracted from the sequence of images may then be combined to form a waveform comprising the one or functional responses of the tissue over time. The waveform, such as the waveform 128, may then be output from the bioreactor 102. In an alternative embodiment, the raw image or frame data is output from the bioreactor 102 and a separate unit—e.g., the control unit 104 or an image analysis unit (not shown)—processes this data to determine the time-series functional response data.

The time-series functional response data produced by the bioreactor 102 (e.g., the waveform 128) provides a high fidelity and high information density representation of a functional response of an engineered tissue over a predetermined time period (e.g., the contractile force produced by a first tissue in response to a pacing frequency of 1 Hz over a 30 second period). To obtain such waveform data from the bioreactor 102 for the engineered tissue 124, a command may be sent to the bioreactor 102 (e.g., from the control unit 104) to begin stimulating the engineered tissue 124 at a pacing frequency. Alternatively, no electrical stimulation is applied so as to observe the spontaneous response of the engineered tissue 124. The sensor assembly 108 then captures a sequence of images of the engineered tissue 124 over the predetermined time period. Decisively, the sequence of images capture the response (e.g., deformation) of the first element 120-1 and/or the second element 120-2 as a result of the contractile response of the engineered tissue 124. The sequence of images is then processed to extract the response of the first element 120-1 and/or the second element 120-2 over the predetermined time period and convert the elements' responses over time to a time-series of functional response over time. For example, the displacement of the first element 120-1 and/or the second element 120-2 may be used to determine the force of the contractile response of the engineered tissue 124. The time-series (e.g., the waveform 128) is then output from the bioreactor 102 for further processing and/or analysis.

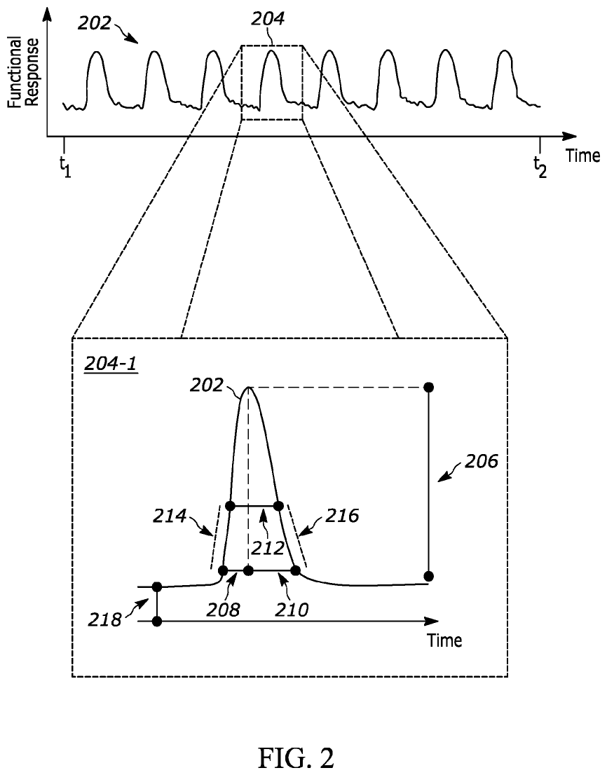

A tissue within the bioreactor 102 (e.g., the engineered tissue 124) may be periodically dosed with a drug or compound and the functional response of the tissue after dosing recorded in the form of a waveform. Alternatively, the functional response of the tissue in the absence of any external dosing regimen may be periodically recorded. In either case, the changes in functional response of the tissue are captured in subsequent waveforms, as illustrated in FIG. 2.

FIG. 2 shows a waveform 202 comprising a functional response of a tissue over a predetermined time period.

The waveform 202 is obtained from a bioreactor such as the bioreactor 102 shown in FIG. 1 as described above. The waveform 202 provides a high fidelity and high information density representation of the functional response of the tissue over the time frame t₁ to t₂. The waveform 202 comprises a plurality of peaks (alternatively referred to as contractions or contraction-relaxation cycles) corresponding to the contractile response of the tissue over the time frame (e.g., 30 s, 40 s, etc.). A magnification of a single contraction-relaxation cycle (i.e., peak) within the portion 204 of the waveform 202 is shown in the expanded portion 204-1.

Typically, a plurality of features are extracted from each contraction-relaxation cycle of the waveform 202 to characterize the waveform 202 and thereby allow for further processing or analysis of the waveform 202. As shown in the expanded portion 204-1, the features extracted from a single contraction-relaxation cycle include peak (or twitch) amplitude 206, time to peak amplitude 208 (or contraction time), time to peak decline 210 (or relaxation time), duration 212 (contraction-relaxation cycle duration or twitch duration), maximum rate of development 214 (or maximum contraction slope), maximum rate of declination 216 (or maximum relaxation slope), and passive tension 218.

Whilst the above features provide a rich characterization of each contraction-relaxation cycle (i.e., of the waveform 202), the noise level of the underlying signal often makes the accurate extraction or measurements of these features difficult. For example, noise introduced into the underlying waveform as a result of measurement (e.g., noise introduced by the sensor assembly 108) or transmission may lead to inaccuracies when identifying key points needed to extract the above features. These inaccuracies in turn limit the effectiveness and results of downstream tasks which utilize such features. Moreover, in vitro functional response data is often limited in volume thereby limiting the applicability of such data to be used to train prediction models such as machine learning regression or classification models.

The present disclosure aims to address some if not all of the above problems by introducing a contraction-relaxation cycle model. As will be described in more detail below, this model is used to perform noise filtering and to enable high-fidelity and high-volume synthetic training data sets to be generated. Such high-fidelity and high-volume synthetic training data sets may then be used to train, update, or otherwise improve artificial intelligence models, such as new or updated machine learning models, in order to produce more accurate and less error prone output. Such output may be used for downstream discovery, development, and/or manufacture of high-quality drug products, such as therapeutics. In some aspects, the present disclosure presents systems and methods for processing of functional response waveforms to enable the efficient and effective extraction of features.

### Systems and Methods for Processing of Functional Response Waveforms.

The present disclosure presents systems and methods for processing of functional response waveforms to enable the efficient and effective extraction of features.

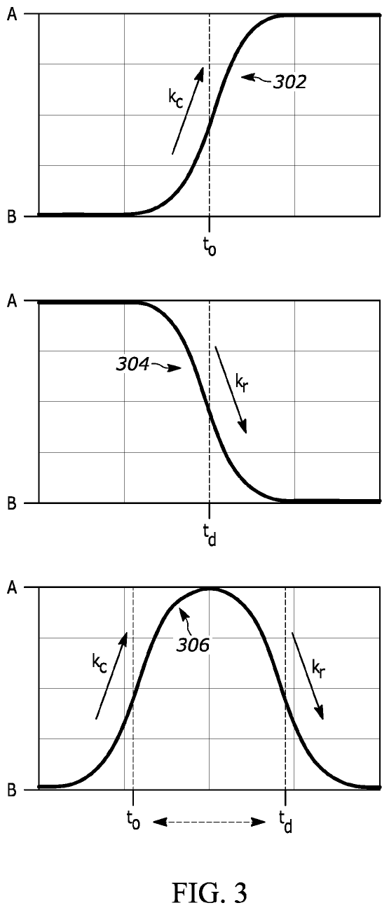

FIG. 3 illustrates elements of a contraction-relaxation cycle model according to an embodiment of the present disclosure. In some aspects, a contraction-relaxation cycle model may comprise a single contraction type contraction-relaxation cycle model

FIG. 3 shows a contraction function 302, a relaxation function 304, and a contraction-relaxation cycle model 306. For brevity, the contraction-relaxation cycle model 306 is also referred to as a single contraction model, single contraction type model, single type model, or single model. The contraction-relaxation cycle model 306 can comprise a combination, or product, of the contraction function 302 and the relaxation function 304. The contraction-relaxation cycle model 306 comprises a combination, or product, of the contraction function 302 and the relaxation function 304. As such, the contraction-relaxation cycle model 306 independently models the contraction response and the relaxation response of the contraction-relaxation cycle of an engineered tissue. Specifically, the growth rate of the contraction response (i.e., the growth rate of the contraction function 302) and the growth, or decay, rate of a relaxation response (i.e., the growth, or decay, rate of the relaxation function 304) vary independently thereby allowing the contraction-relaxation cycle model 306 to model a large variety of different response types. The contraction-relaxation cycle model 306 is therefore able to be used effectively and efficiently across a range of application areas involving varying types of functional response waveforms. Additionally, or alternatively, the growth rate of a contraction response (i.e., the growth rate of the contraction function 302) and the growth, or decay, rate of single relaxation response (i.e., the growth, or decay, rate of the relaxation function 304) vary independently thereby allowing the single contraction model 306 to model a large variety of single contraction-relaxation responses.

According to an aspect of the present disclosure, a contraction-relaxation cycle model, such as the contraction-relaxation cycle model 306, can be fit to in vitro functional response data of engineered tissue such as a waveform of contractile force of an engineered cardiac tissue. A noise filtered representation of the in vitro functional response data is then generated from the contraction-relaxation cycle model and features are accurately extracted from the noise filtered representation.

The contraction function and the relaxation function are logistic (sigmoid) functions, biexponential functions, or any other suitable functions. In one embodiment, logistic functions are used to model force based functional response waveforms whilst biexponential functions are used to model calcium transients based functional response waveforms. In general, the contraction-relaxation-cycle model (e.g., the contraction-relaxation cycle model 306) comprises a product of the contraction function, f_{c}(t), and the relaxation function, fᵣ(t):

$${f}_{m} ( t ) = A * ( {f}_{c} ( t ) * {f}_{r} ( t ) ) + B . \qquad ( 1 )$$

Here, A is the maximum value of the contraction-relaxation cycle model and B is a shift of the contraction-relaxation cycle model applied along the y-axis. In the embodiment shown in FIG. 3, the contraction function 302 is a rising logistic function of the form:

$${f}_{c} ( t ) = \frac{1}{1 + {e}^{- ( t - {t}_{0} ) ⋆ \frac{1}{{k}_{c}}}} , \qquad ( 2 )$$

and the relaxation function 304 is a falling logistic function of the form:

$${f}_{r} ( r ) = 1 - \frac{1}{1 + {e}^{- ( t - {t}_{d} ) * \frac{1}{{k}_{r}}}} . \qquad ( 3 )$$

The contraction-relaxation cycle model 306 may thus be expressed as:

$${f}_{m} ( t ) = A * ( \frac{1}{1 + {e}^{- ( 𝔱 - {t}_{0} ) ⋆ \frac{1}{{k}_{c}}}} * ( 1 - \frac{1}{1 + {e}^{- ( 𝔱 - {t}_{d} ) ⋆ \frac{1}{{k}_{r}}}} ) ) + B . \qquad ( 4 )$$

Here, t₀ and k_{c} parameterize the contraction function 302 and correspond to the midpoint of the contraction function 302 and the growth rate (inverse of logistic growth rate) of the contraction function 302 accordingly. The parameters t_{d} and kᵣ parameterize the relaxation function 304 and correspond to the midpoint of the relaxation function 304 and the growth, or decay, rate (inverse of logistic growth, or decay, rate) of the relaxation function 304 accordingly.

The set of parameters, θ=[A, B, t₀, t_{d}, k_{c}, k_{y}], thus fully describes a contraction-relaxation-cycle of an engineered tissue thereby allowing the contraction-relaxation cycle model 306 to be fit to, and approximate, contraction-relaxation cycle(s) within real world functional response data obtained from an engineered tissue (e.g., artificial cardiac tissue).

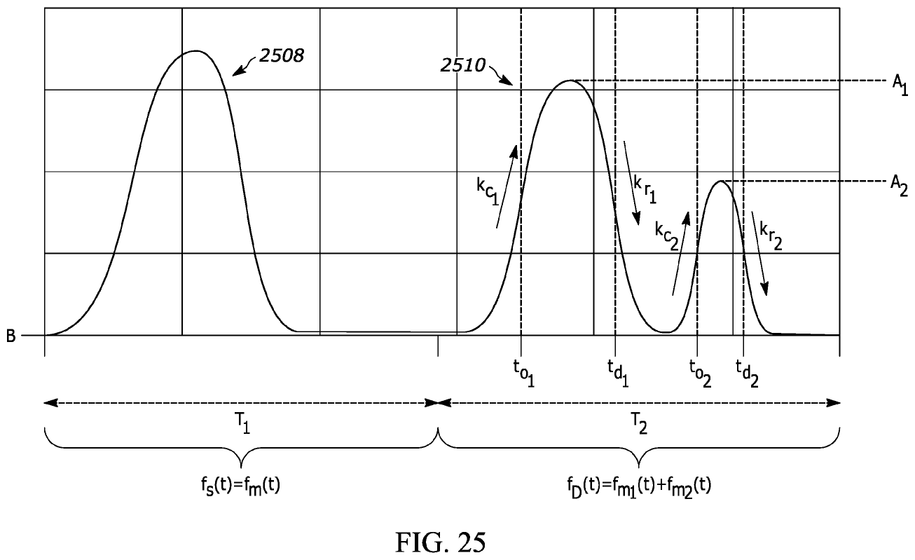

Whilst the contraction-relaxation cycle model illustrated in FIG. 3 can be used to model functional responses having a single contraction type, not all functional responses follow a single contraction type. For example, arrhythmias such as atrial fibrillation or tachycardia may cause ectopic beats resulting in irregular beating patterns. Therefore, functional response waveforms may comprise a combination of both single contraction types, as described in relation to FIG. 3 above, and double contraction types, as described in relation to FIG. 25 below.

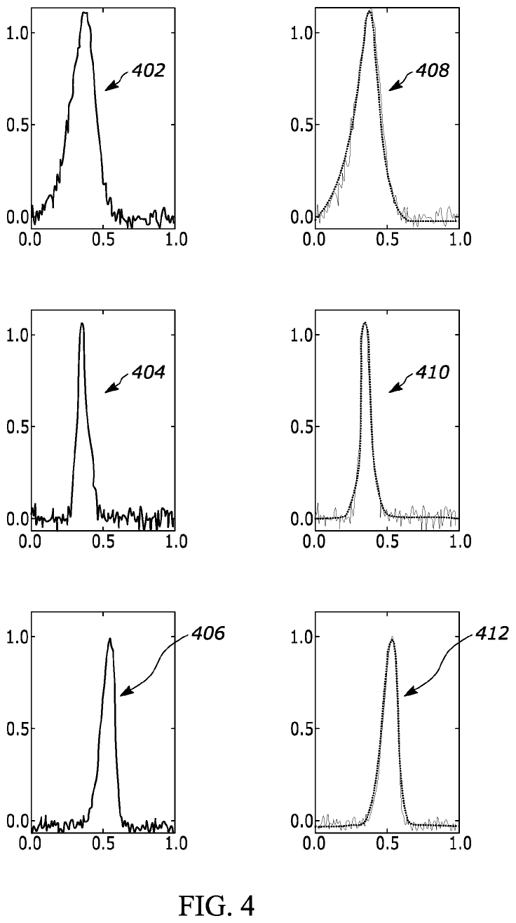

FIG. 4 shows three example waveforms with corresponding contraction-relaxation cycle model fits.

FIG. 4 shows a first waveform 402, a second waveform 404, and a third waveform 406. FIG. 4 further shows a first model fit waveform 408, a second model fit waveform 410, and a third model fit waveform 410. Each of the model fit waveforms correspond to waveforms generated from a contraction-relaxation cycle model to one of the first, second, or third waveforms. For example, the first model fit waveform 408 corresponds to the waveform generated from a contraction-relaxation cycle model fit to the first waveform 402.

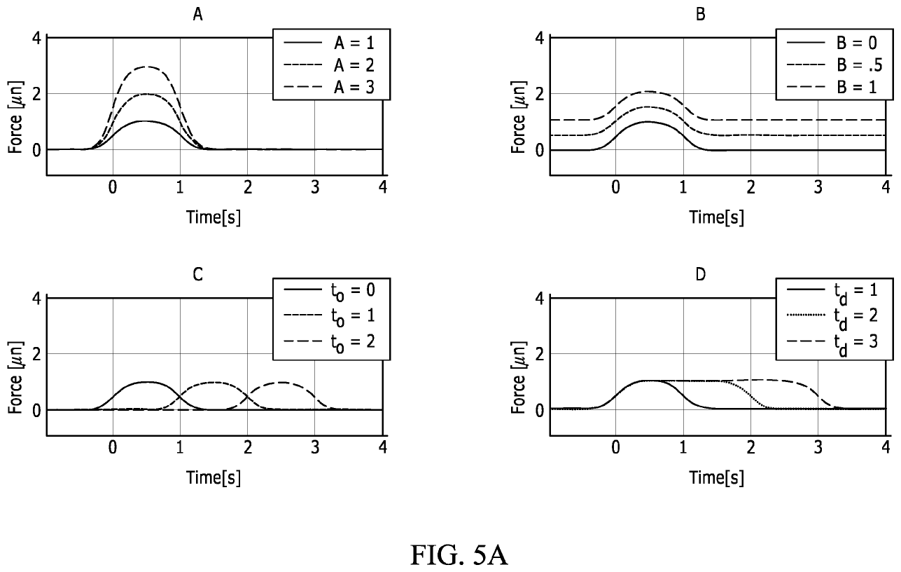

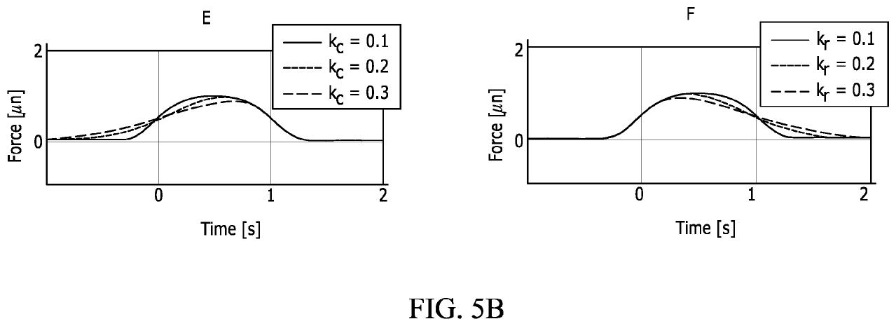

FIGS. 5A and 5B show the effect of each parameter of a contraction-relaxation cycle model according to an embodiment of the present disclosure.

Each plot in FIGS. 5A and 5B show a contraction-relaxation cycle model (e.g., single contraction type contraction-relaxation cycle model) according to Equation (4) above with one varying parameter. In some aspects, as stated above, the parameter changes of the single contraction-relaxation cycle models shown in FIGS. 5A and 5B can also apply to parameter changes of a double contraction type model, as described, for example, in relation to FIG. 25 below.

Plot A of FIG. 5A shows the contraction-relaxation cycle model with varying values for the maximum value parameter, A. Plot B of FIG. 5A shows the contraction-relaxation cycle model with varying values for the shift parameter, B. Plot C of FIG. 5A shows the contraction-relaxation cycle model with varying values for the contraction midpoint parameter, t₀. Plot D of FIG. 5A shows the contraction-relaxation cycle model with varying values for the relaxation midpoint parameter, t_{d}. Plot E of FIG. 5B shows the contraction-relaxation cycle model with varying values for the contraction growth rate parameter, k_{c}. Plot F of FIG. 5B shows the contraction-relaxation cycle model with varying values for the relaxation growth rate parameter, kᵣ.

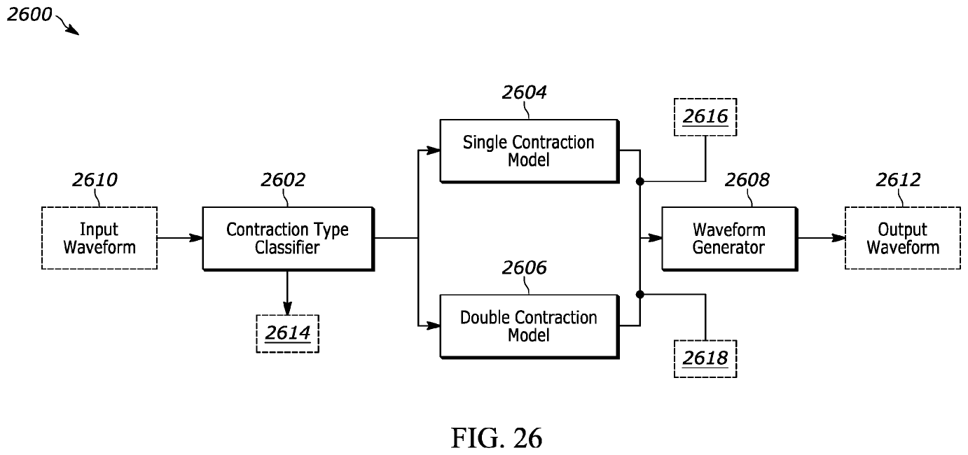

The range and variations in shape which can be captured by both or either of the single and/or double contraction type contraction-relaxation cycle means that the model(s) can be accurately fit to a range of different functional response data. In particular, complex responses can be efficiently and accurately modelled by independently modelling the contraction response and the relaxation response(s), e.g., in some cases independently modelling the growth and decay of one or both responses within a given cycle(s). The contraction-relaxation model(s) of the present disclosure can thus be fit to a range of different functional response data by identifying the contraction type within the functional response data to determine which contraction type model to fit, and/or, additionally or alternatively, determining the parameters which best fit the model to the underlying data. One example of this process is shown for FIG. 26, as described herein.

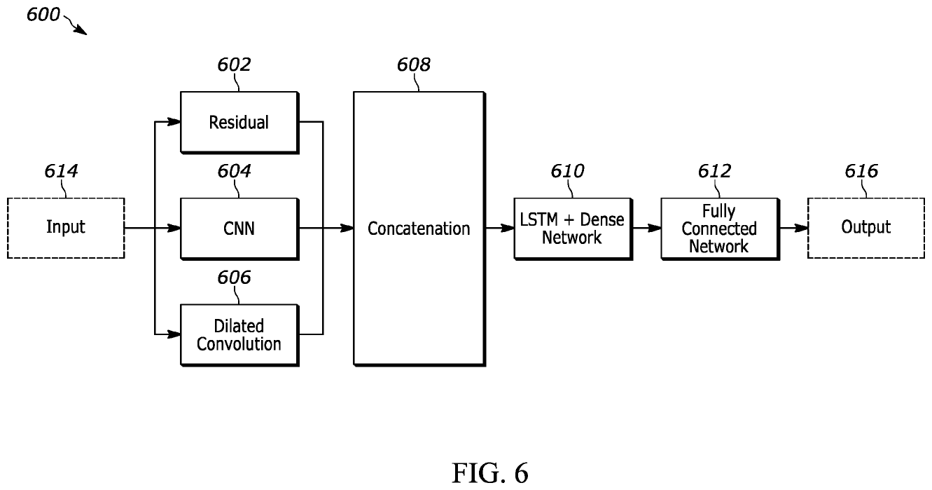

As a further example, the contraction-relaxation model can be fit using any suitable parameter fitting technique such as least squares based fitting, or machine learning based fitting such as that shown in FIG. 6 or elsewhere herein.

FIG. 6 shows a parameter estimation model 600 for fitting parameters to a contraction-relaxation cycle model according to an embodiment of the present disclosure.

The parameter estimation model 600 comprises a machine learning model in the form of a deep neural network. The parameter estimation model 600 comprises a residual layer 602, a convolutional neural network 604, a dilated convolution network 606, a concatenation block 608, a long short-term memory (LSTM) and network 610, and a fully connected network 612. The parameter estimation model 600 receives an input vector 614 and produces an output vector 616.

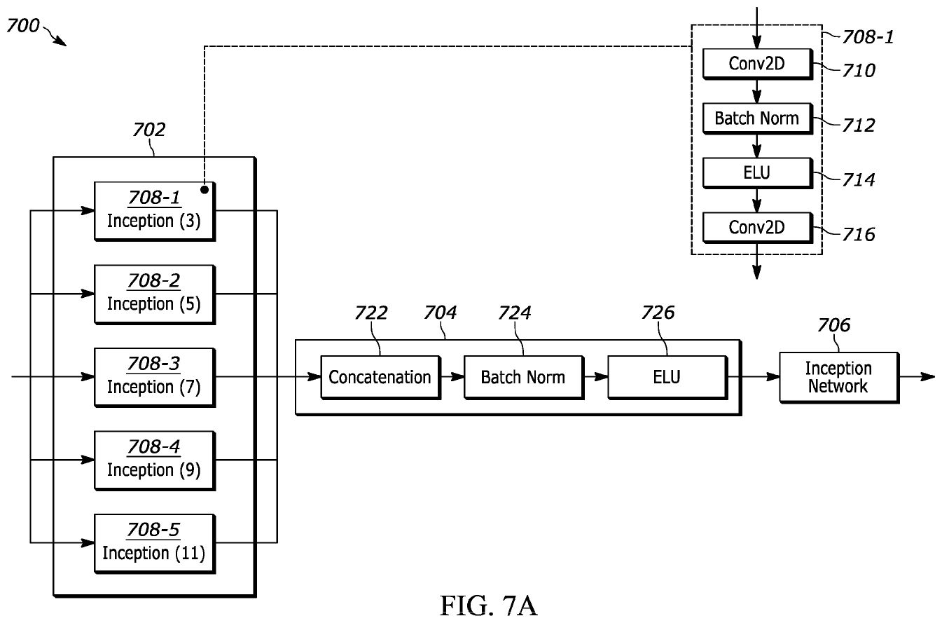

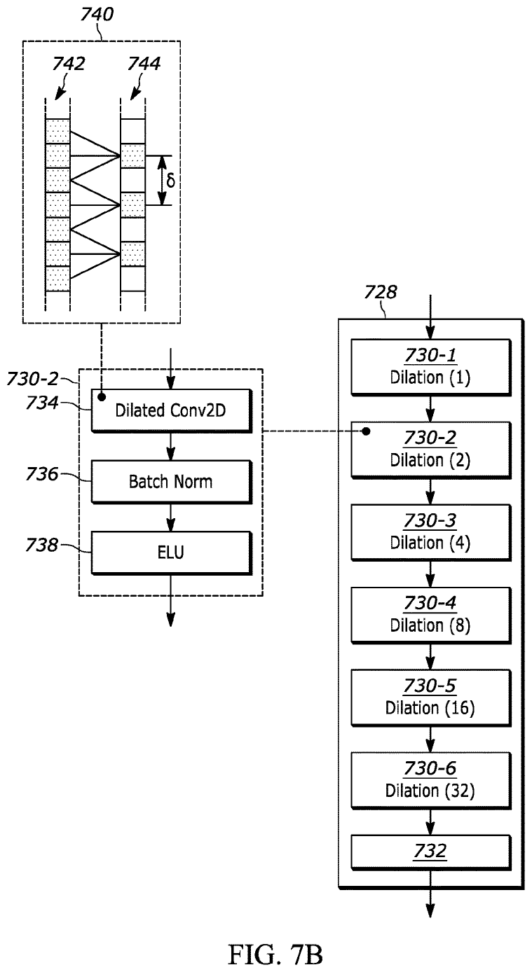

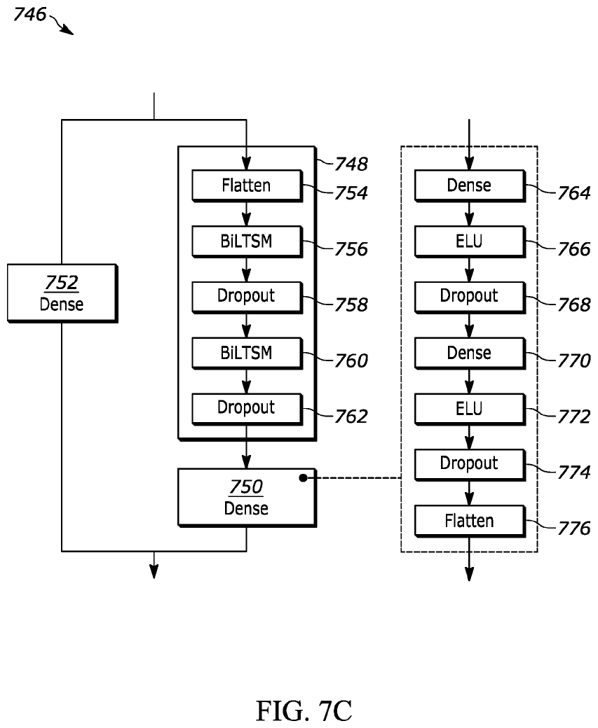

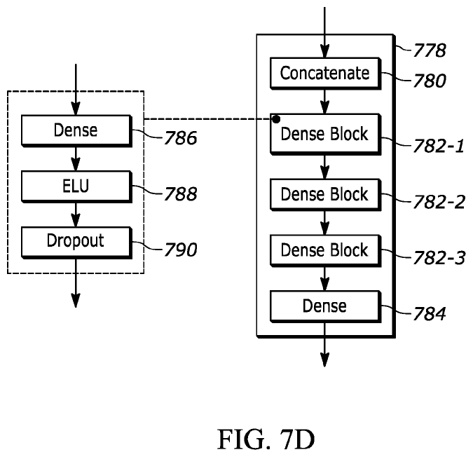

The input vector 614 comprises a sequence (time-series) corresponding to a waveform comprising a contraction response and a relaxation response of an artificial tissue during a single contraction-relaxation cycle (e.g., the waveform 202 shown in FIG. 2). The input vector 614 is fed to the residual layer 602, the convolutional neural network 604, and the dilated convolution network 606. The residual layer 602 is a 2-dimensional (2D) convolution layer with 64 filters of size 1. The convolutional neural network 604 and the dilated convolution network 606 are described in more detail in relation to FIGS. 7A and 7B below. The outputs of each of the residual layer 602, the convolutional neural network 604, and the dilated convolution network 606 are concatenated at the concatenation block 608. The concatenation block 608 comprises a depth concatenation layer which concatenates the outputs of the residual layer 602, the convolutional neural network 604, and the dilated convolution network 606, a batch normalization layer that normalizes the output of the depth concatenation layer, and an exponential linear unit layer that performs the identity operation on positive inputs and performs α(exp(x)−1) for inputs x that are negative, where α=1. The output of the concatenation block 608 (i.e., the output of the exponential linear unit layer) is fed to the LSTM and network 610, which is described in more detail in relation to FIG. 7C below. The output of the LSTM and network 610 is fed to the fully connected network 612 which is described in more detail in relation to FIG. 7D below. The output vector 616 produced by the fully connected network 612 comprises parameter values for the contraction-relaxation-cycle model fit to the waveform represented in the input vector 614.

Beneficially, by using dilated convolution and an LSTM network, the parameter estimation model 600 is efficiently and accurately able to fit parameters to a waveform comprising a full contraction-relaxation cycle. This efficiency and accuracy leads to more efficient use of computing resources and improved performance of downstream tasks, such as drug discovery and development, which utilize the estimated parameters. The parameter estimation model also reduces error as would otherwise be present in the original waveform, for example, as produced by the underlying computing device. The reduced error parameter estimation model thereby operates more efficiency, reducing additional computational cycles for an underlying computing device (e.g., one or more processors and/or memory of an underly device), thus saving processor and memory utilization of the device upon which the parameter estimation model is executed.

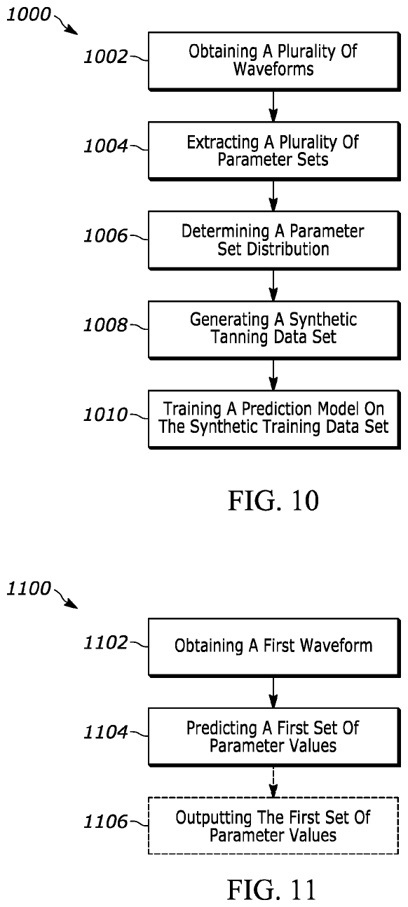

In one implementation, the parameter estimation model 600 is trained using a synthetic data set comprises 40,000 training samples and 10,000 validation samples. The synthetic data set is generated using the approach described in relation to FIG. 10 below. Minibatch gradient descent with a batch size of 128 is used with an ADAM solver to train the parameter estimation model 600. The ADAM solver has an initial learning rate of 1e-3 with early stopping based on validation loss.

Additionally, or alternatively, in some embodiments FIG. 6 parameter estimation model 600 may comprise a prediction model. In such embodiments parameter estimation model 600 may be referred to herein as prediction model 600.

In such aspects, prediction model 600 comprises a machine learning model in the form of a deep neural network. The prediction model 600 comprises a residual layer 602, a convolutional neural network 604, a dilated convolution network 606, a concatenation block 608, a long short-term memory (LSTM) network 610, and a fully connected network 612. The prediction model 600 receives an input vector 614 and produces an output vector 616.

The prediction model 600 is trained for different purposes. As described in more detail below, by modifying the fully connected network 612, the prediction model 600 is trained either to predict a contraction type or predict the parameters of a contraction-relaxation cycle model. In the case of predicting model parameters, the prediction model 600 is trained to predict either single contraction type or double contraction type model parameters. Therefore, the prediction model 600 shown in FIG. 6 and described in detail in relation to FIGS. 7A-7D can be trained as a parameter estimation model or a contraction type classifier.

The input vector 614 comprises a sequence (time-series) corresponding to a waveform comprising at least one contraction response and at least one relaxation response of an artificial tissue. The waveform represented by the input vector 614 has a contraction type—either a single contraction type or a double contraction type. For the single contraction type, the input vector 614 comprises a time-series corresponding to a single contractile response. For the double contraction type, the input vector comprises a time-series corresponding to a double contractile response.

The input vector 614 is fed to the residual layer 602, the convolutional neural network 604, and the dilated convolution network 606. In one implementation, the residual layer 602 is a 2-dimensional (2D) convolution layer with 64 filters of size 1. The convolutional neural network 604 and the dilated convolution network 606 are described in more detail in relation to FIGS. 7A and 7B below. The outputs of each of the residual layer 602, the convolutional neural network 604, and the dilated convolution network 606 are concatenated at the concatenation block 608. The concatenation block 608 comprises a depth concatenation layer which concatenates the outputs of the residual layer 602, the convolutional neural network 604, and the dilated convolution network 606, a batch normalization layer that normalizes the output of the depth concatenation layer, and an exponential linear unit layer that performs the identity operation on positive inputs and performs α(exp(x)−1) for inputs x that are negative. In one implementation, α=1. The output of the concatenation block 608 (i.e., the output of the exponential linear unit layer) is fed to the LSTM network 610, which is described in more detail in relation to FIG. 7C below. The output of the LSTM network 610 is fed to the fully connected network 612 which is described in more detail in relation to FIG. 7D below.

As described in more detail below, the architecture of the fully connected network 612, as well as the final activation function, is modified depending on the application of the prediction model 600. Consequently, the output vector 616 produced by the fully connected network 612 comprises a vector or scalar score associated with a contraction type classification when the prediction model 600 is used for contraction type classification. For example, the output vector 616 may comprise a probability that the waveform represented by the input vector 614 is a single contraction type or a double contraction type. When the prediction model 600 is used for parameter estimation, the output vector 616 produced by the fully connected network 612 comprises a vector of parameter values for the single or double contraction-relaxation cycle model fit to the waveform represented by the input vector 614.

Beneficially, by using dilated convolution and an LSTM network, the prediction model 600 is able to predict accurately the contraction type of a waveform and efficiently fit parameters to a waveform comprising either a single or double contraction type. These benefits lead to more efficient use of computing resources and improved performance of downstream tasks, such as drug discovery and development.

In one implementation, the prediction model 600 is trained for the task of contraction type classification using a training data set of labelled waveforms. A training data set of approximately 10,000 training samples and approximately 2,000 validation samples is used. The training data set comprises approximately 6,000 manually labelled single contraction type contraction-relaxation cycle waveforms and 6,000 manually labelled double contraction type contraction-relaxation cycle waveforms.

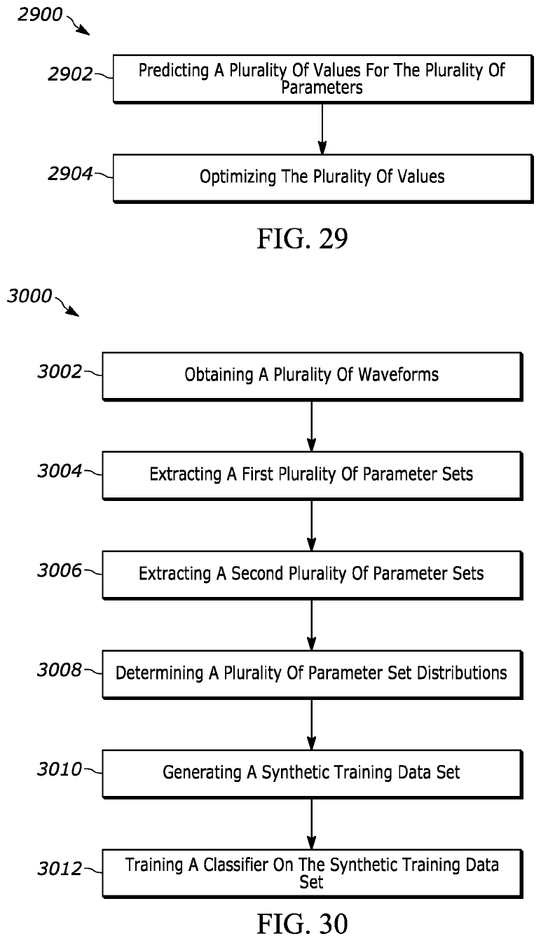

In another implementation, the prediction model 600 is trained using a synthetic data set comprising 40,000 training samples and 10,000 validation samples. The synthetic data set is generated using the approach described in relation to FIG. 29 below. When training the prediction model 600 for contraction type classification, the synthetic data set comprises 25,000 single contraction type waveforms and 25,000 double contraction type waveforms. For the task of contraction type classification, each waveform within the synthetic data set is associated with a corresponding label indicating whether the waveform is a single contraction type or a double contraction type. When training the prediction model 600 for parameter estimation, the synthetic data comprises either single contraction type waveforms or double contraction type waveforms along with the corresponding parameters for each waveform. Thus, for parameter estimation one model is trained to predict the parameters for single contraction type waveforms and another model is trained to predict the parameters for double contraction type waveforms.

For both the classification and the parameter estimation tasks in both of the above implementations, minibatch gradient descent with a batch size of 128 is used with an ADAM solver to train the prediction model 600. The ADAM solver has an initial learning rate of 1e-3 with early stopping based on validation loss.

FIG. 7A shows a convolutional neural network 700 which forms part of the parameter estimation model 600 (e.g., a prediction model) of FIG. 6 according to an embodiment of the present disclosure.

The convolutional neural network 700 corresponds to the convolutional neural network 604 of the parameter estimation model 600 (e.g., a prediction model) shown in FIG. 6. The convolutional neural network 700 comprises a first inception network 702, a concatenation block 704, and a second inception network 706. The first inception network 702 comprises a first inception module 708-1, a second inception module 708-2, a third inception module 708-3, a fourth inception module 708-4, and a fifth inception module 708-5. Each of these inception modules comprises the same architecture, as illustrated in relation to the first inception module 708-1 which comprises a first 2D convolution layer 710, a batch normalization layer 712, an exponential linear unit layer 714, and a second 2D convolution layer 716. The concatenation block 704 comprises a depth concatenation layer 722, a batch normalization layer 724, and an exponential linear unit layer 726 with α=1. The second inception network 706 has the same architecture (i.e., the same number and configuration of inception modules) as the first inception network 702.

Each inception module in the first inception network 702 and the second inception network 706 shares the same architecture (i.e., as illustrated in relation to the first inception module 708-1 shown in FIG. 7A) and is parameterized according to the filter size of the second 2D convolution layer (e.g., the second 2D convolution layer 716). The filter sizes of the second 2D convolution layer in the first inception module 708-1, the second inception module 708-2, the third inception module 708-3, the fourth inception module 708-4, and the fifth inception module 708-5 are [3,5, 7, 9, 11] respectively. The first 2D convolution layer (e.g., the first 2D convolution layer 710) comprises 64 filters of size 1. The batch normalization layer 712 comprises a standard batch normalization layer and the exponential linear unit layer 714 comprises a standard exponential linear unit layer with α=0.1.

FIG. 7B shows a dilated convolution network 730 which forms a part of the parameter estimation model 600 (e.g., a prediction model) of FIG. 6 according to an embodiment of the present disclosure.

The dilated convolution network 730 corresponds to the dilated convolution network 606 of the parameter estimation model 600 (e.g., a prediction model) shown in FIG. 6. The dilated convolution network 730 comprises a first dilated convolution module 730-1, a second dilated convolution module 730-2, a third dilated convolution module 730-3, a fourth dilated convolution module 730-4, a fifth dilated convolution module 730-5, a sixth dilated convolution module 730-6, and a final dilated 2D convolution layer 732.

The architecture of each dilated convolution module is the same and is illustrated in FIG. 7B by the architecture of the second dilated convolution module 730-2 which comprises a dilated 2D convolution layer 734, a batch normalization layer 736, and an exponential linear unit layer 738 with α=0.1. Each dilated convolution module is parameterized by the size of the dilation operation performed at the dilated 2D convolution layer. The dilation sizes of the dilated 2D convolution layer for the first dilated convolution module 730-1, the second dilated convolution module 730-2, the third dilated convolution module 730-3, the fourth dilated convolution module 730-4, the fifth dilated convolution module 730-5, and the sixth dilated convolution module 730-6 are [1, 2, 4, 8, 16, 32] respectively.

The dilation operations are illustrated in the first expanded portion 740 which shows a portion 742 of the input to the second dilated convolution module 730-2 and a portion 744 of the dilated 2D convolution layer 734. Here, the input corresponds to the output of the exponential linear unit layer of the first dilated convolution module 730-1. Dilated convolution beneficially increases the receptive field (i.e., the time period which the layer processes) of the layer without increasing the number of parameters. The dilated 2D convolution layer 734 performs this expansion by inserting zeroes between each filter element. As shown in the portion 744 of the dilated 2D convolution layer 734, a dilation size or factor of 2 results in a single zero to be padded between each filter element (whereas, for a dilation size or factor of 4, three zeroes would be inserted).

The final dilated 2D convolution layer 732 corresponds to a standard dilated 2D convolution layer with a dilation size or factor of 64.

FIG. 7C shows a long short-term memory (LSTM) network 746 which forms part of the parameter estimation model 600 (e.g., a prediction model) of FIG. 6 according to an embodiment of the present disclosure.

The LSTM network 746 corresponds to the LSTM network 610 of the parameter estimation model 600 (e.g., a prediction model) shown in FIG. 6. The LSTM network 746 comprises an LSTM module 748, a first dense module 750, and a second dense module 752. The LSTM network 746 comprises a flattening layer 754, a first bidirectional LSTM layer 756, a first dropout layer 758, a second bidirectional LSTM layer 760, and a second dropout layer 762. The first dense module 750 comprises a first dense layer 764, a first exponential linear unit layer 766 with α=1, a first dropout layer 768, a second dense layer 770, a second exponential linear unit layer 772 with α=1, a second dropout layer 774, and a flattening layer 776. The architecture of the second dense module 752 is the same as the first dense module 750.

The LSTM module 748 seeks to learn, via the bidirectional LSTM layers, bidirectional long-term dependencies between the time steps of the waveform (i.e., time-series) data. As such, the LSTM module 748 is able to learn from the complete waveform at each time step. Both the first bidirectional LSTM layer 756 and the second bidirectional LSTM layer 760 comprise 64 hidden units. The first dropout layer 758 and the second dropout layer 762 help to reduce overfitting by randomly setting inputs to zero with a probability of 0.1.

The first dense layer 764 and the second dense layer 770 of the first dense module 750 have an output size of 256. The first dropout layer 768 and the second dropout layer 774 randomly set inputs to zero with a probability of 0.5. As stated above, the architecture of the second dense module 752 is the same as the architecture of the first dense module 750.

FIG. 7D shows a fully connected network 778 which forms part of the parameter estimation model 600 (e.g., a prediction model) of FIG. 6 according to an embodiment of the present disclosure.

The fully connected network 778 corresponds to the fully connected network 612 of the parameter estimation model 600 (e.g., a prediction model) shown in FIG. 6. The fully connected network 778 comprises a concatenation layer 780, a first dense block 782-1, a second dense block 782-2, a third dense block 782-3, a dense layer 784, and/or an output layer 786.

The first dense block 782-1, the second dense block 782-2, and the third dense block 782-3 all comprise the same architecture. As shown in relation to the first dense block 782-1, the architecture comprises a dense layer 786, an exponential linear unit layer 788 with α=1, and a dropout layer 790. The dense layer of the first dense block 782-1 and the second dense block 782-2 have output sizes of 512. The dense layer of the third dense block 782-3 has an output size of 1024. The dropout layers of the first dense block 782-1 and the second dense block 782-2 randomly set inputs to zero with a probability of 0.5. The dropout layer of the third dense block 782-3 randomly sets inputs to zero with a probability of 0.1.

In one example, the dense layer 784 has an output size corresponding to the number of parameters that the parameter estimation model is to fit. The output size is set to 6 to learn the full set of parameters, 0=[A, B, t₀, t_{d}, k_{c}, kᵣ], of the contraction-relaxation cycle model described above in relation to FIG. 3. Therefore, the output of the dense layer 784 corresponds to the output of the parameter estimation model (e.g., the output vector 616 of the parameter estimation model 600 shown in FIG. 6).

In one embodiment, the estimated parameters produced by the parameter estimation model are further refined using an optimization approach. As will be described in more detail below in relation to FIG. 9, the optimization approach utilized comprises a simplex search algorithm such as the Nelder-Mead method.

Once the contraction-relaxation cycle model has been fit to the data, the waveform can be reconstructed from the model to generate a noise filtered representation of the waveform. For example, a number of points (e.g., 100, 200, 500, 1000, etc.) are sampled from the contraction-relaxation cycle model fit to the data over the time period of the first waveform to which the contraction-relaxation cycle model is fit. Beneficially, this allows high-resolution waveform data to be generated from the contraction-relaxation cycle which helps ensure that more accurate features of the functional response of the engineered tissue are extracted. This in turn helps improve the accuracy and efficacy of downstream tasks involving the functional response features. The contraction-relaxation cycle model also reduces error as would otherwise be present in the original waveform, for example, as produced by the underlying computing device. The reduced error contraction-relation cycle model thereby operates more efficiency, reducing additional computational cycles for an underlying computing device (e.g., one or more processors and/or memory of an underly device), thus saving processor and memory utilization of the device upon which the contraction-relation cycle model is executed.

In another example, when the parameter estimation model 600 (e.g., the prediction model 600), of which the fully connected network 778 forms a part, is used for contraction type classification, the dense layer 784 has two nodes-one for each contraction type such that the output for each node corresponds to the probability that the input waveform is of the respective contraction type. When the prediction model 600 is used for parameter estimation, the dense layer 784 has an output size (i.e., number of nodes) corresponding to the number of parameters that the parameter estimation model is to fit. For single contraction type models, the output size (i.e., the number of nodes in the dense layer 784) is set to 6 to learn the full set of parameters, 0=[A, B, t₀, t_{d}, k_{c}, k_{y}], of the single contraction type contraction-relaxation cycle model described above in relation to FIG. 3. For double contraction type models, the output size (i.e., the number of nodes in the dense layer 784) is set to 11 to learn the full set of parameters, 0=[A₁, t₀_{₁}, t_{d}_{₁}, k_{c}_{₁}, kᵣ_{₁}, A₂, t₀_{₂}, t_{d}_{₂}, k_{c}_{₂}, kᵣ_{₂}, B], of the double contraction type contraction-relaxation cycle model described above in relation to FIG. 25.

The output of the dense layer 784 corresponds to the output of the parameter estimation model (e.g., the output vector 616 of the prediction model 600 shown in FIG. 6).

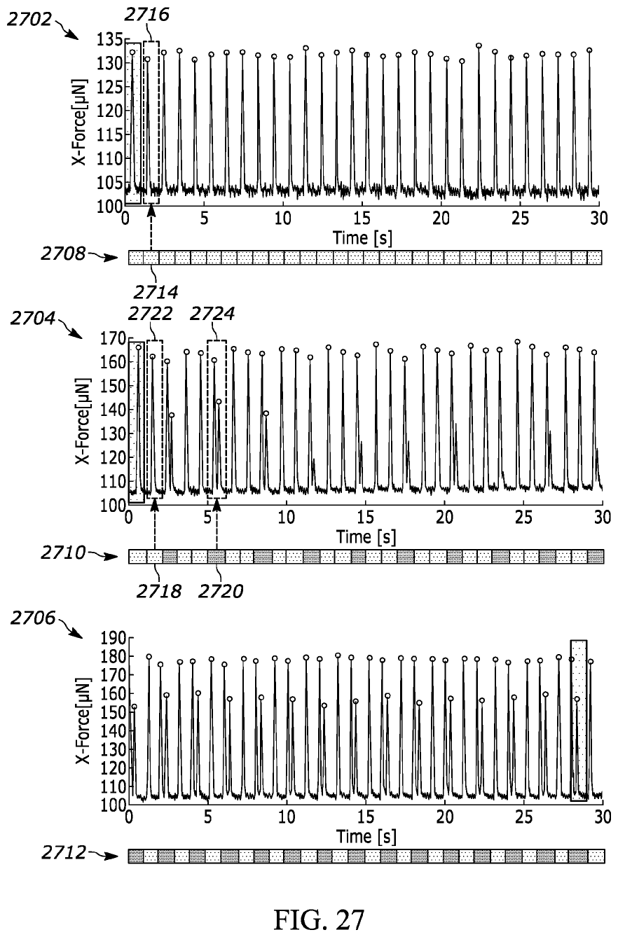

When the prediction model described above (e.g., the prediction model 600 of FIG. 6) is used for contraction type classification, the predicted contraction type is used to determine which of the two parameter estimation models to fit to the waveform (as illustrated by the system 2600 in FIG. 26). In one embodiment, the predicted contraction type provides a phenotype for the state of the engineered tissue (e.g., a disease state, a treatment state, etc.), as illustrated in FIG. 27.

The description will now turn to methods for utilizing the above described contraction-relaxation cycle model for processing of functional response waveforms.

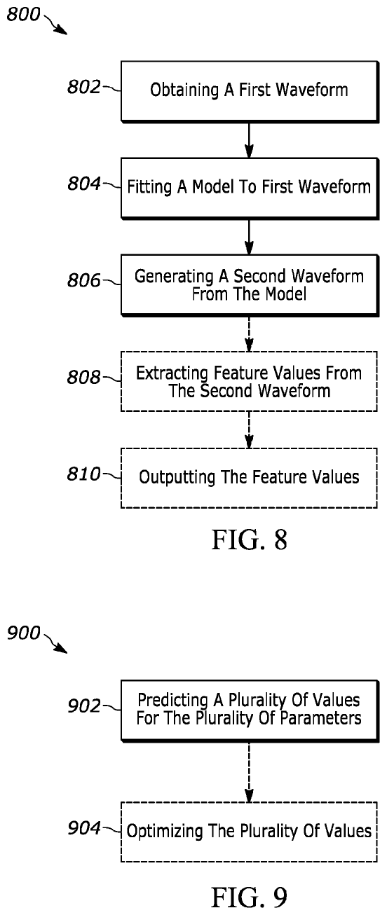

FIG. 8 shows a method 800 for processing a functional response waveform according to an aspect of the present disclosure.

The method 800 comprises the steps of obtaining 802 a first waveform, fitting 804 a model to the first waveform, and generating 806 a second waveform from the model. The method 800 further comprises the optional steps of extracting 808 feature values from the second waveform and outputting 810 the feature values. In some aspects, the extracted feature values may be used to train a machine learning model, where the machine learning model would have an increased predictive accuracy by using feature values of the second waveform, which itself has filtered error prone data (e.g., noise), leading to improved predictive accuracy. A prediction may then be generated by inputting a data set of a human tissue into the machine learning model. The prediction may define one or more characteristics of the human tissue and may correspond to at least one of the one or more features value. In this way, highlight accurate machine learning, or otherwise artificial intelligence models, may be trained or generated using feature values of a reduced error waveform (e.g., the second waveform). In one embodiment, the method 800 is performed by the model fitting unit 104-1 of the control unit 104 shown in FIG. 1.

In general, the method 800 is used to generate a noise filtered, or noise suppressed, representation of a contraction-relaxation cycle waveform (functional response waveform). In embodiments, the contraction-relaxation cycle waveform is obtained from hardware such as a bioreactor (e.g., the bioreactor 102 shown in FIG. 1) which may introduce noise into the waveform due to factors such as sensor variability, signal transmission, signal conversion, and the like. The contraction-relaxation cycle waveform thus comprises a potentially noisy representation of the functional response of an engineered tissue during a single contraction-relaxation cycle. Beneficially, the method 800 efficiently generates a noise filtered representation of the single contraction-relaxation—cycle thereby improving the accuracy of the extracted features which in turn helps improve the performance of downstream tasks which utilize such features.

At the step of obtaining 802, a first waveform comprising a contraction response and a relaxation response of an artificial tissue during a single contraction-relaxation cycle is obtained.

The first waveform (i.e., a single contraction-relaxation cycle waveform, a single cycle waveform, a twitch cycle waveform, a peak, or a functional response waveform) captures the functional response of the artificial tissue during a single contraction-relaxation cycle which comprises a contraction period (i.e., the period within which the artificial tissue contracts and generates tension) and a relaxation period (i.e., the period within which the artificial tissue returns to its normal state, or length). As such, the contraction response of the first waveform comprises a functional response of the artificial tissue during a contraction period of the contraction-relaxation cycle, and the relaxation response comprises a functional response of the artificial tissue during a relaxation period of the single contraction-relaxation cycle. The functional response is a contractile force of the artificial tissue (e.g., a contractile force as measured using data obtained from a sensor assembly 108 of a bioreactor 102 within which the artificial tissue is grown or sustained). Alternatively, the functional response is a contractile displacement, a transient calcium response, or a change in membrane potential.

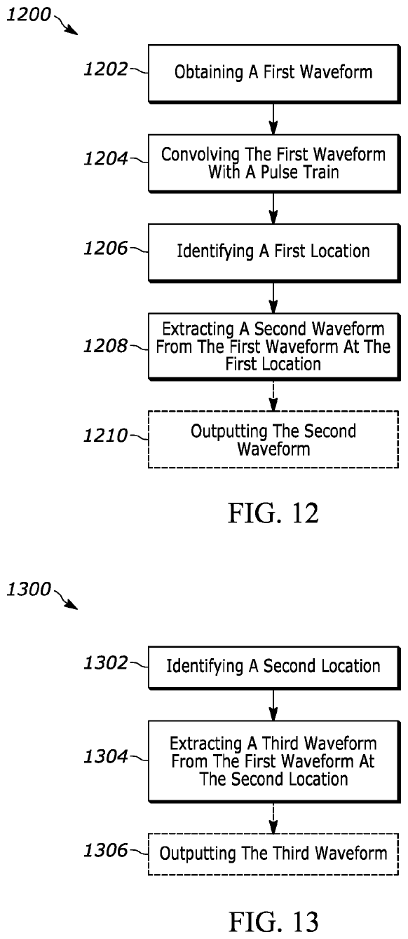

The artificial tissue, or engineered tissue, comprises engineered muscle tissue such as engineered cardiac tissue or engineered skeletal muscle tissue. In one embodiment, the first waveform is obtained from bioreactor (e.g., the bioreactor 102 shown in FIG. 1) comprising the artificial tissue. As such, the first waveform is obtained from a waveform, obtained from the bioreactor, which comprises a plurality of contraction-relaxation cycles of the artificial tissue. In one embodiment, the first waveform is obtained, or extracted, from the waveform using an extraction method such as method 1200 described in more detailed below in relation to FIG. 12.

At the step of fitting 804, a model is fit to the first waveform. The model, or contraction-relaxation cycle model, independently parameterizes growth of the contraction response and the relaxation response. As such, the model does not assume that the underlying functional response is symmetrical. This allows the model to be fit efficiently and accurately to a range of functional responses from a large variety of engineered tissue types. More efficient and accurate model fitting helps to generate more accurate features which in turn leads to better data being generated.

The model comprises a contraction function, f_{c}(t), and a relaxation function, fᵣ(t). In one embodiment, the contraction function is a rising logistic function having a positive growth rate and the relaxation function is a falling logistic function having a negative growth rate. The contraction response of the contraction-relaxation cycle is thus modeled by the rising logistic function and the relaxation response of the contraction-relaxation-cycle is modeled by the falling logistic function. The model comprises a product of the rising logistic function and the falling logistic function.

The contraction-relaxation cycle model comprises a plurality of parameters associated with the contraction response and the relaxation response. The plurality of parameters comprises a maximum value parameter, A, a rate of rise parameter, k_{c}, a rate of fall parameter, kᵣ, a y-shift parameter, B, a rising x-shift parameter, t₀, and a falling x-shift parameter, t_{d}. The relation of each of these parameters to the overall model are shown and described in more detail in relation to FIGS. 5A and 5B above.

In one embodiment, the model is fit to the first waveform using a prediction of parameter values obtained from a machine learning model (e.g., the parameter estimation model shown in FIG. 6), as described in more detail in relation to the method 900 of FIG. 9 below.

At the step of generating 806, a second waveform is generated from the model fit to the first waveform such that the second waveform comprises a noise filtered representation of the first waveform. For example, a number of points (e.g., 100, 200, 500, 1000, etc.) are sampled from the contraction-relaxation cycle model fit to the data over the time period of the first waveform to which the contraction-relaxation cycle model is fit. Beneficially, this allows high-resolution waveform data to be generated from the contraction-relaxation cycle which helps ensure that more accurate features of the functional response of the engineered tissue are extracted. This in turn helps improve the accuracy and efficacy of downstream tasks involving the functional response features.

Optionally, the second waveform may be output. In one embodiment, outputting the second waveform comprises storing, or saving, the second waveform to a persistent storage such as a non-volatile memory, a non-transitory medium, or the like. Additionally, or alternatively, outputting the second waveform comprises transmitting the second waveform via a network (e.g., a local area network, a wide area network, and the like), or displaying the waveform for review by a user.

At the optional step of extracting 808, one or more feature values are extracted from the second waveform.

The second waveform is a noise filtered representation of the first waveform (i.e., a noise filtered representation of the contraction-relaxation cycle) and, as such, enables more accurate values of features of the underlying functional response to be extracted. The one or more feature values include one or more of a twitch, or peak, amplitude value (i.e., the peak amplitude 206 shown in FIG. 2), a contraction time value (i.e., the time to peak amplitude 208 shown in FIG. 2), a maximum contraction slope value (i.e., the maximum rate of development 214 shown in FIG. 2), a relaxation time value (i.e., the time to peak decline 210 shown in FIG. 2), a maximum relaxation slope value (i.e., the maximum rate of declination 216 shown in FIG. 2), and a twitch duration value (i.e., the duration 212 shown in FIG. 2).

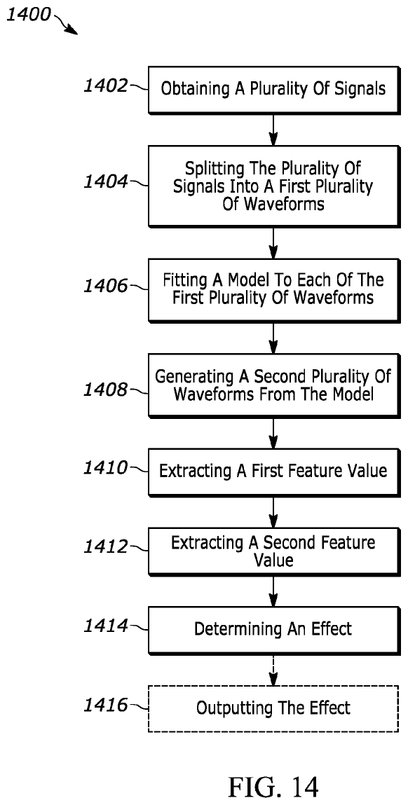

Once extracted, the one or more feature values may be used as a quantitative descriptor of the contraction-relaxation cycle and thus provide a numeric representation of the functional response of the artificial tissue during the contraction-relaxation cycle. As will be described in more detail in relation to FIG. 14, such features are used in various downstream processing tasks such as effect identification in drug discovery and development.

At the optional step of outputting 810, the one or more feature values extracted from the second waveform are output. In one embodiment, outputting the one or more feature values comprises storing, or saving, the one or more feature values to a persistent storage such as a non-volatile memory, a non-transitory medium, or the like. Additionally, or alternatively, outputting the one or more feature values comprises transmitting the one or more feature values via a network (e.g., a local area network, a wide area network, and the like), or displaying the one or more feature values for review by a user. FIG. 9 shows a method 900 for fitting a model to a functional response waveform according to an embodiment of the present disclosure.

In one embodiment, the method 900 is performed as part of the fitting 804 step of the method 800. The method 900 comprises the step of predicting 902 a plurality of values for the plurality of parameters and further comprises the optional step of optimizing 904 the plurality of values.

The steps in the method 900 are used to predict parameter values for the contraction-relaxation cycle model from the first waveform. A trained machine learning model (e.g., the parameter estimation model shown in FIG. 6) is used to predict the parameter values such that the fit model closely approximates the first waveform.

At the step of predicting 902, a plurality of values are predicted for the plurality of parameters of the model such that the model fit to the first waveform comprises the plurality of values for the plurality of parameters.

At the optional step of optimizing 904, the plurality of values determined at the predicting 902 step are optimized by minimizing an error between the first waveform and the model fit to the first waveform (i.e., using the plurality of values for the plurality of parameters of the model).

Given a plurality of values for the model, θ̅, determined at the step of predicting 902, an updated plurality of values, θ̅′, are sought at the step of optimizing 904 such that L_{θ̅′}<L_{θ̅}. Here, L_{θ̅}(⋅) is a cost, or loss, function which measures the error between the first waveform, X₁, and the model, f_{θ}, fit to the first waveform according to the set of parameter values θ. A lower value of L indicates a better fit of the model to the first waveform. In one embodiment, the cost function, L, is the root mean square error:

$${L}_{θ} = \sqrt{𝔼 ( {( {f}_{θ} - {X}_{1} )}^{2} )} . \qquad ( 5 )$$

Which in one embodiment corresponds to:

$${L}_{θ} = \sqrt{\frac{1}{N} ( {\sum }_{i - 1}^{N} {( {f}_{θ} ( i ) - {x}_{i} )}^{2} )} . \qquad ( 6 )$$

The optimization of θ is a multidimensional problem because the model involves multiple parameters. Minimizing L_{θ} therefore requires the simultaneous fitting of multiple parameters. To perform this minimization, in one embodiment the plurality of values are optimized using a simplex search algorithm such as the Nelder-Mead method. Beneficially, performing optimization after obtaining an initial prediction of the parameters helps obtain an accurate model fit whilst making better use of processing resources because the optimization process starts at a solution which is expected to be close to an optimal solution.

FIG. 10 shows a method 1000 shows a method for training a parameter estimation model using synthetic training data according to an aspect of the present disclosure.

The method 1000 comprises the steps of obtaining 1002 a plurality of waveforms, extracting 1004 a plurality of parameter sets, determining 1006 a parameter set distribution, generating 1008 a synthetic training data set, and training 1010 a prediction model on the synthetic training data set. In one embodiment, the method 1200 is performed by the control unit 104, or a sub-unit thereof, shown in FIG. 1.

In many situations, the efficacy of prediction models (e.g., machine learning models such as deep neural networks) are limited by the amount and quality of training data available. In the absence of high-volume, high-quality, training data, prediction models will often fail to produce adequate outputs. In the present disclosure, this problem would lead to inaccurate contraction-relaxation models being fit which would subsequently reduce the effectiveness and applicability of such models being used in practice for tasks such as drug discovery and development. The method 1000 seeks to address such problems by generating high-fidelity synthetic training data thereby allowing an almost limitless amount of data to be generated. This helps to improve the performance of the prediction model being trained which subsequently improves the accuracy of the models fit using the prediction model. This improvement of accuracy helps drive improvements to downstream tasks which utilize features extracted from such models.

At the step of obtaining 1002, a plurality of waveforms are obtained. The plurality of waveforms comprise functional responses of one or more artificial tissues during a single contraction-relaxation cycle.

The plurality of waveforms correspond to the real, or bootstrap, data from which the synthetic training data set will be generated. Each waveform corresponds to a time-series of values which represent the functional response (e.g., contractile force, calcium transients, etc.) of an artificial, or engineered, tissue over a single contraction-relaxation cycle. Therefore, each waveform contains a contraction period and a relaxation period and may be parameterized according to the contraction-relaxation model described above in relation to FIG. 3.

To help ensure that the synthetic training data is representative of as wide a population of contraction-relaxation cycles as possible, the plurality of waveforms are preferably obtained from a variety of artificial tissues across a range of different conditions. The artificial tissues comprise one or more engineered muscle tissues such as engineered cardiac tissue and/or engineered skeletal muscle tissue. Variety is achieved by obtaining waveforms from artificial tissues across a range of different cell lines, disease states, and treatments. Alternatively, the scope of conditions within the plurality of waveforms is restricted thereby allowing the synthetic data, and the subsequent parameter estimation model, to be finetuned to specific applications. For example, the plurality of waveforms may be restricted to vehicular treated waveforms to generate a control parameter estimation model or may be restricted to specific tissue types (e.g., engineered cardiac tissue) to generate a tissue-specific parameter estimation model. Beneficially, this helps improve the performance of the parameter estimation model when it is known which type of waveform the parameter estimation model will be used for.

At the step of extracting 1004, a plurality of parameter sets are extracted from the plurality of waveforms. A parameter set of the plurality of parameter sets characterizes a corresponding waveform of the plurality of waveforms.

A parameter set comprises the parameters of the contraction-relaxation cycle model (e.g., as described above in relation to FIG. 3). In one embodiment, a parameter set associated with a waveform comprises a maximum value parameter value (A), a shift parameter value (B), a contraction midpoint parameter value (t₀), a contraction growth rate parameter value (k_{c}), a relaxation midpoint parameter value (t_{d}), and a relaxation growth rate parameter value (kᵣ).

The plurality of parameter sets are extracted using either a supervised, unsupervised, or semi-supervised approach. According to the supervised approach, a parameter set is fit manually to each waveform. For example, a first waveform is presented to a user and the values of the parameters within the parameter set are adjusted by the user until a second waveform produced by a contraction-relaxation cycle model fit according to the parameter set closely matches the waveform. The final parameter set which results in the closely matching second waveform is then used as a parameter set within the plurality of parameter sets associated with the first waveform. According to the unsupervised approach, a parameter set is automatically fit to a waveform (e.g., using the approach described in relation to FIGS. 8 and 9 above). In one embodiment, the unsupervised approach utilizes a trained machine learning model to predict parameter set values for a waveform. According to the semi-supervised approach, the automatically determined parameter sets obtained according to the unsupervised approach are manually reviewed and refined by one or more users.

At the step of determining 1006, a parameter set distribution is determined from the plurality of parameter sets.

The plurality of parameter sets extracted at the extracting 1004 step of the method 1000 comprise a plurality of values for each parameter of the contraction-relaxation cycle model. For example, if 100 parameters sets are extracted, then there will be 100 parameter values extracted for each parameter of the contraction-relaxation cycle model. At the step of determining 1006, a distribution, or distribution of values, is determined for each parameter of the model. In one embodiment, a distribution is independently determined for each parameter. Alternatively, a multivariate distribution is determined for the plurality of parameters forming the parameter set.

Any suitable method may be used to determine the parameter set distribution such as histogram based approaches, density estimation approaches, and clustering approaches.

In one embodiment, the parameter set distribution is determined using a kernel density estimation (KDE) method which utilizes a kernel and a bandwidth parameter to estimate the parameter set distribution. In one implementation, a normal (Gaussian) kernel is used with the bandwidth selected using either cross-validation or a bandwidth selection approach such as Scott's rule or Silverman's rule.

Once the parameter set distribution has been determined, a parameter set can be obtained by sampling from this distribution (i.e., sampling from each individual distribution or sampling from the joint distribution).

At the step of generating 1008, a synthetic training data set is generated. Each element of the synthetic training data set comprises a synthetic waveform and a corresponding parameter set used to generate the synthetic waveform. The corresponding parameter set is obtained from the parameter set distribution.

The synthetic training data set is generated by repeatedly sampling a parameter set for the contraction-relaxation cycle model from the parameter set distribution (as described above) and generating a corresponding waveform for each of the sampled parameter sets.

In this way, a training data set of any size (e.g., 1000, 10000, 100000, etc. training data elements) can be generated efficiently. The synthetic data will also closely approximate real waveform data because the parameter set distribution is modelled on real world data.

Optionally, a noise component is added to each of the waveforms in the synthetic training data set. The noise component is determined via a uniform distribution which is determined from the plurality of waveforms.

At the step of training 1010, a prediction model is trained using the synthetic training data set. The prediction model is trained to estimate an output parameter set from an input waveform. In one embodiment, the prediction model corresponds to the parameter estimation model 600 described in relation to FIG. 6 above. As stated above, training the parameter estimation model 600 in one embodiment comprises using minibatch gradient descent with a batch size of 128 and an ADAM solver. The ADAM solver has an initial learning rate of 1e-3 with early stopping based on validation loss. More details regarding the training of the parameter estimation model 600 performed at the step of training 1010 is given above in relation to the description of FIG. 6.

FIG. 11 shows a method 1100 for predicting a set of parameter values for a contraction-relaxation cycle model using a synthetically trained prediction model according to an embodiment of the present disclosure.

The method 1100 comprises the steps of obtaining 1102 a first waveform and predicting 1104 a first set of parameter values. The method 1100 also comprises the optional step of outputting 1106 the first set of parameter values. In one embodiment, the method 1100 is performed by the control unit 104 shown in FIG. 1, or a sub-unit therefore such as the model fitting unit 104-1.

At the step of obtaining 1102, a first waveform is obtained. The first waveform comprises a functional response of a first artificial tissue.

At the step of predicting 1104, a first set of parameter values are predicted from the first waveform using a prediction model trained on a synthetically generated training data set (as described above in relation to FIG. 10).

At the optional step of outputting 1106, the first set of parameter values are output. In one embodiment, outputting the first set of parameter values comprises storing, or saving, the first set of parameter values to a persistent storage such as a non-volatile memory, a non-transitory medium, or the like. Additionally, or alternatively, outputting the first set of parameter values comprises transmitting the first set of parameter values via a network (e.g., a local area network, a wide area network, and the like), or displaying the first set of parameter values for review by a user.

The methods described in relation to FIGS. 8-10 above fit a contraction-relaxation cycle model to a waveform comprising a single functional response (i.e., a single contraction-relaxation cycle) of an artificial tissue. In practice, waveforms typically contain multiple contraction-relaxation cycles which may require multiple model fits. To analyze and process such waveforms, each individual contraction-relaxation cycle is extracted from the waveform prior to model fitting.

FIG. 12 shows a method 1200 for extracting a contraction-relaxation cycle waveform according to an aspect of the present disclosure.

The method 1200 comprises the steps of obtaining 1202 a first waveform, convolving 1204 the first waveform with a pulse train, identifying 1206 a first location, and extracting 1208 a second waveform from the first waveform at the first location. The method 1200 also comprises the optional step of outputting 1210 the second waveform. In one embodiment, the method 1200 is performed by the signal processing unit 104-2 of the control unit 104 shown in FIG. 1.

In general, the method 1200 extracts a single contraction-relaxation cycle waveform (i.e., a single cycle, twitch cycle, peak, or functional response) from a larger waveform comprising multiple contraction-relaxation cycles. The larger waveform may be obtained from a hardware device such as a bioreactor (i.e., the bioreactor 102 shown in FIG. 1) and will typically correspond to the functional response of an artificial tissue under certain conditions. For example, the larger waveform may comprise the contractile force response of an engineered cardiac tissue stimulated at 1 Hz over a period of 30 seconds. In this example, the larger waveform will comprise approximately 30 peaks, or 30 contraction-relaxation cycles, corresponding to the contractions of the engineered cardiac tissue in response to the electrical stimulation. The method 1200 provides an efficient and accurate mechanism for extracting each contraction-relaxation cycle from the larger waveform such that these sub-waveforms may then be used for further processing and analysis (e.g., fitting a contraction-relaxation cycle model to these waveforms and extracting relevant features as described in relation to FIG. 8 above).

At the step of obtaining 1202, a first waveform is obtained. The first waveform comprises a plurality of functional responses of an artificial tissue stimulated at a first frequency.

In one embodiment, the first waveform is obtained from a bioreactor (e.g., the bioreactor 102 shown in FIG. 1) in which the artificial, or engineered, tissue is grown/maintained. As stated previously, the artificial tissue comprises engineered muscle tissue such as engineered cardiac tissue or engineered skeletal muscle tissue. As described above in relation to FIG. 1, an electrical stimulation is applied to a cell culture during maturation and, once matured, an electrical stimulation is applied to the artificial tissue to simulate a physiological environment that is native to the artificial tissue thereby allowing the functional response of the artificial tissue to this stimulation to be measured. The first waveform obtained at the step of obtaining 1202 comprises the functional responses of the artificial tissue in response to stimulation at the first frequency. In one embodiment, the first frequency, or pacing frequency, at which the artificial tissue is stimulated is from 0.1 Hz to 20 Hz. In a further embodiment, the first frequency is from 1 Hz to 6 Hz.

Optionally, the method 1200 comprises the step of stimulating (not shown) the artificial tissue at the first frequency prior to the step of obtaining 1202 the first waveform. For example, an instruction (e.g., the instruction 126 in FIG. 1), or command, is sent to the bioreactor containing the artificial tissue (e.g., the bioreactor 102 in FIG. 1) to cause the bioreactor to stimulate the artificial tissue at the first frequency.

At the step of convolving 1204, the first waveform is convolved with a pulse-train to generate a convolved waveform. The pulse-train is generated at the first frequency.

The pulse-train corresponds to an idealized representation of the functional responses of the artificial tissue at the first frequency. As is known, a pulse-train, or pulse wave, is a waveform comprising non-sinusoidal (rectangular) pulses, or waves, of duration T with a frequency of 1/T, where T₀ is the period of the pulse-train. The duty cycle of the pulse-train is thus T/T₀. The pulse-train with which the first waveform is convolved therefore comprises a sequence of rectangular pulses with period 1/f and duration T where f is the first frequency.

Optionally, the method 1200 further comprises the step of generating (not shown) the pulse-train at the first frequency prior to performing the step of convolving 1204.

Given the first waveform, X₁, and the pulse-train, p, the convolution, y=(X₁+p), performed at the step of convolving 1204 is:

$$y [ k ] = ( {X}_{1} * p ) [ k ] = {\sum }_{n} {X}_{1} [ n - k ] p [ k ] . \qquad ( 7 )$$

At the step of identifying 1206, a first location associated with a maximum value of the convolved waveform is identified. The first location corresponds to an expected location of a first contraction-relaxation cycle.

The maximum value of the convolved waveform corresponds to the location where the first waveform and the pulse-train best align. As such, the location of the maximum value of the convolved waveform is used to identify the most likely location of a single contraction-relaxation cycle within the first waveform. The location of the maximum value of the convolved waveform also provides an anchor point from which the other contraction-relaxation cycles can be extracted from the first waveform.

At the step of extracting 1208, a second waveform is extracted from the first location of the first waveform. The second waveform comprises the first contraction-relaxation cycle and has a first duration proportional to the first frequency.

The first location identified at the identifying 1206 step corresponds to the most likely location of a single contraction-relaxation cycle within the first waveform. The second waveform extracted from the first location thus comprises this contraction-relaxation cycle. Because the first waveform corresponds to the functional response of the artificial tissue when stimulated at a predetermined, set, frequency, the duration, or length, of the second waveform is proportional to this frequency. For example, if the artificial tissue was stimulated at 1 Hz then the first duration would be 1 s, if the artificial tissue was stimulated at 2 Hz then the first duration would be 0.5 s, etc.

Thus, the second waveform corresponds to a window, or subframe, within the first waveform having a length corresponding to the first duration. In one embodiment, the second waveform is centered at the first location such that a midpoint of the second waveform is aligned, or substantially aligned, to the first location of the first waveform.

At the optional step of outputting 1210, the second waveform is output. In one embodiment, outputting the second waveform comprises storing, or saving, the second waveform to a persistent storage such as a non-volatile memory, a non-transitory medium, or the like. Additionally, or alternatively, outputting the second waveform comprises transmitting the second waveform via a network (e.g., a local area network, a wide area network, and the like), or displaying the waveform for review by a user.

In one embodiment, outputting the second waveform comprises causing the second waveform to be output to another process or method of the present disclosure. For example, the second waveform may be output to the method 800 described above such that the step of obtaining 802 comprises obtaining the second waveform from the method 1200.

FIG. 13 shows a method 1300 for extracting a further contraction-relaxation cycle from a waveform according to an embodiment of the present disclosure.

The method 1300 comprises the steps of identifying 1302 a second location, extracting 1304 a third waveform from the first waveform at the second location, and further comprises the optional step of outputting 1306 the third waveform. The method 1300 is performed after the method 1200. Particularly, the method 1300 may be performed after the step of identifying 1206 the first location and may be performed in parallel to the step of extraction 1208 the second waveform. In one embodiment, the method 1300 is performed by the signal processing unit 104-2 of the control unit 104 shown in FIG. 1.

The method 1300 is used to extract a further contraction-relaxation cycle waveform from the first waveform. Beneficially, the extraction performed at the method 1300 is efficient and highly parallel because the method 1300 leverages prior information regarding the expected locations of the contraction-relaxation cycles within the first waveform thus enabling the contraction-relaxation cycles to be extracted independently.

At the step of identifying 1302, a second location is identified based on the first location and the first frequency. The second location corresponds to an expected location of a second contraction-relaxation cycle.

The first location (identified at the identifying 1206 step of the method 1200) corresponds to the best alignment between the first waveform and the pulse-train. As such, the first location may be understood as the most likely location of a contraction-relaxation cycle within the first waveform. Because the first waveform comprises functional responses of the artificial tissue at a predetermined frequency (i.e., the first frequency), the other contraction-relaxation cycles, which are linked to the functional responses of the artificial tissue, are highly likely to be located at locations spaced from the first location. The first location can thus act as an anchor point within the first waveform from which the other contraction-relaxation cycle waveforms can be extracted.

The second location corresponds to an expected location of a second contraction-relaxation cycle and will be spaced from the first location by a distance proportional to the first frequency. Specifically, given the first location, t₁, within the first waveform, the second location, t₂, will be t₂=t₁+(a×T₀) where a is a step factor and T₀=1/f is the period of the pulse-train. Therefore, the next contraction-relaxation cycle can be identified by setting the scaling factor to a=1 and the previous contraction-relaxation cycle can be identified by setting the scaling factor to a=−1.

At the step of extracting 1304, a third waveform is extracted from the second location of the first waveform. The third waveform comprises the second contraction-relaxation cycle and has a second duration proportional to the first frequency.

The third waveform corresponds to a functional response (i.e., contraction-relaxation cycle) of the artificial tissue when stimulated at a predetermined, set, frequency. Therefore, the duration, or length, of the third waveform is proportional to this frequency. For example, if the artificial tissue was stimulated at 1 Hz then the second duration would be 1 s, if the artificial tissue was stimulated at 2 Hz then the second duration would be 0.5 s, etc. In one embodiment, the first duration and the second duration are the same.

In one embodiment, the third waveform is centered at the second location such that a midpoint of the third waveform is aligned, or substantially aligned, to the second location of the first waveform.

At the optional step of outputting 1306, the third waveform is output. In one embodiment, outputting the third waveform comprises storing, or saving, the third waveform to a persistent storage such as a non-volatile memory, a non-transitory medium, or the like.

Additionally, or alternatively, outputting the third waveform comprises transmitting the third waveform via a network (e.g., a local area network, a wide area network, and the like), or displaying the waveform for review by a user.

In one embodiment, outputting the third waveform comprises causing the third waveform to be output to another process or method of the present disclosure. For example, the third waveform may be output to the method 800 described above such that the step of obtaining 802 comprises obtaining the third waveform from the method 1300.

As stated above, the method 1300 of extracting a further contraction-relaxation cycle waveforms from the first waveform may be repeated for all contraction-relaxation cycles within the first waveform. Because the extraction performed by the method 1300 depends only on the first location and the first frequency, no further signal processing or analysis is required to identify the locations of the further contraction-relaxation cycles. The method 1300 therefore provides a fast and efficient method for extracting contraction-relaxation cycles from a waveform. These waveforms can then be processed further, e.g., by fitting a model to the waveform to generate a noise filtered representation of the waveform.

FIG. 14 shows a method 1400 for predicting a treatment effect using a contraction-relaxation cycle model according to an aspect of the present disclosure.

The method 1400 comprises the steps of obtaining 1402 a plurality of signals, splitting 1404 the plurality of signals into a first plurality of waveforms, fitting 1406 a model to each of the first plurality of waveforms, generating 1408 a second plurality of waveforms from the model, extracting 1410 a first feature value, extracting 1412 a second feature value, and determining 1414 an effect. The method 1400 also comprises the optional step of outputting 1416 the effect. In one embodiment, the method 1400 is performed by the control unit 104, or a sub-unit thereof, shown in FIG. 1.

In general, the method 1400 describes an application of the contraction-relaxation cycle model to a downstream drug discovery/development task. Specifically, the contraction-relaxation cycle model is used to generate accurate feature values efficiently from a baseline signal and a perturbation signal of an engineered tissue. Accurately extracting features from these signals allows an effect associated with the perturbation to be efficiently and accurately identified.

At the step of obtaining 1402, a plurality of signals are obtained. The plurality of signals comprise a baseline signal and a perturbation signal. The baseline signal comprises a first plurality of functional responses of an engineered tissue under reference conditions. The perturbation signal comprises a second plurality of functional responses of the engineered tissue under perturbed conditions involving a first perturbation.

The baseline signal and the perturbation signal comprise a plurality of functional responses (i.e., a plurality of contraction-relaxation cycles, or peaks) of the engineered tissue under reference and perturbation conditions. In general, reference conditions refer to conditions which provide a baseline comparison to the perturbation conditions. In embodiments, a reference condition is a condition associated with a control setup or environment. A reference condition may correspond to an engineered, or artificial, tissue in its default, natural, or unaltered state (i.e., without dosage of a drug or agent). Alternatively, a reference condition may correspond to vehicle treated engineered tissue. Perturbation conditions refer to conditions in which the engineered tissue has been perturbed in some way. Examples of perturbation conditions include the administration of a drug or compound (i.e., a perturbant), a disease state, a different cell line, or a physical perturbance applied to the engineered tissue. As such, perturbation conditions may alternatively be referred to as treatment conditions. In the case of perturbations involving a drug or compound, the conditions are further associated with an effect related to the drug or compound such as a mechanism of action or a toxicity. Given the range of different perturbation conditions, an engineered tissue may be associated with more than one perturbation condition (e.g., a diseased engineered tissue having been treated with a specific compound).

In one embodiment, the baseline signal and the perturbation signal are obtained from a bioreactor (e.g., the bioreactor 102 of FIG. 1) in which the engineered, or artificial, tissue is grown/maintained. As stated previously, the artificial tissue comprises engineered muscle tissue such as engineered cardiac tissue or engineered skeletal muscle tissue. The baseline signal and the perturbation signal are obtained at two different time points. For example, the baseline signal is obtained at a first time point, the engineered tissue is then perturbed according to the first perturbation (e.g., a first dosage of a compound is applied to the engineered tissue), and the perturbation signal is obtained at a second time point subsequent the first time point. The baseline signal and the perturbation signal comprise the functional response of the engineered tissue when stimulated at a predetermined pacing frequency (e.g., 0.1 Hz, 0.5 Hz, 1 Hz, 2 Hz, etc.). Alternatively, the baseline signal and the perturbation signal comprise the spontaneous functional response of the engineered tissue in the absence of external stimulation.

At the step of splitting 1404, the plurality of signals are split into a first plurality of waveforms. Each waveform of the first plurality of waveforms comprises a contraction response and a relaxation response of the engineered tissue during a single contraction-relaxation cycle.

The plurality of signals are split into a first plurality of waveforms using the method 1200 for extracting a contraction-relaxation cycle waveform described above. Alternatively, the plurality of signals are split by manually annotating or extracting the regions within the first plurality of waveforms which correspond to individual contraction-relaxation cycles.

The first plurality of waveforms generated at the step of splitting 1404 comprise a first subset of waveforms associated with waveforms extracted from the baseline signal and a second subset of waveforms associated with waveforms extracted from the perturbation signal. The subsequent model fitting (described below) is independent of the source of the waveform (i.e., independent of whether the waveform is a reference of perturbation waveform), but the identification of whether a waveform corresponds to reference or perturbation condition is used in the subsequent feature extraction steps.

At the step of fitting 1406, a model is fit to each waveform of the first plurality of waveforms. The model is a contraction-relaxation cycle model which independently parameterizes growth of the contraction response and the relaxation response of the engineered tissue during the single contraction-relaxation cycle of each waveform.

The step of fitting 1406 the model to each waveform corresponds to the step of fitting 804 described in more detail above in relation to FIG. 8. Consequently, at the step of fitting 1406, the step of fitting 804 is repeated for each waveform in the first plurality of waveforms. The process of fitting a model to a waveform is described in more detail above in relation to FIGS. 8 and 9.

At the step of generating 1408, a second plurality of waveforms are generated from the model fit to each waveform of the first plurality of waveforms. The second plurality of waveforms comprise a plurality of filtered baseline waveforms associated with the baseline signal and a plurality of filtered treatment waveforms associated with the treatment signal. The step of generating 1408 corresponds to repeatedly applying the step of generating 806 (described in more detail above in relation to FIG. 8) to the model fit to each waveform in the first plurality of waveforms. The process of generating a second waveform from a model fit to a first waveform is described in more detail above in relation to FIGS. 8 and 9 above.

At the step of extracting 1410, a first feature value of a first feature is extracted from the plurality of filtered baseline waveforms.

The plurality of filtered baseline waveforms comprise noise filtered, or noise suppressed, representations of the contraction-relaxation cycles in the baseline signal. Because the signals have been filtered to remove noise, features can be accurately extracted from these waveforms.

The step of extracting 1410 the first feature value comprises extraction a plurality of feature values from the plurality of filtered baseline waveforms such that the first feature value comprises the plurality of feature values, or a representation of the plurality of feature values. As such, a value of the first feature is extracted from each of the plurality of filtered baseline waveforms to determine to first feature value. In one embodiment, the first feature value comprises an average (mean, median, etc.) value of the first feature determined from the plurality of filtered baseline waveforms. In a further embodiment, the first feature value comprises a maximum value, minimum value, or distribution of values determined from the plurality of filtered baseline waveforms.

The first feature is one of a twitch, or peak, amplitude (i.e., the peak amplitude 206 shown in FIG. 2), a contraction time (i.e., the time to peak amplitude 208 shown in FIG. 2), a maximum contraction slope (i.e., the maximum rate of development 214 shown in FIG. 2), a relaxation time (i.e., the time to peak decline 210 shown in FIG. 2), a maximum relaxation slope (i.e., the maximum rate of declination 216 shown in FIG. 2), or a twitch duration (i.e., the duration 212 shown in FIG. 2).

At the step of extracting 1412, a second feature value of the first feature is extracted from the plurality of filtered perturbation waveforms.

The step of extracting 1412 the second feature value comprises extraction a plurality of feature values from the plurality of filtered perturbation waveforms such that the second feature value comprises the plurality of feature values, or a representation of the plurality of feature values. As such, a value of the first feature is extracted from each of the plurality of filtered perturbation waveforms to determine to second feature value. In one embodiment, the second feature value comprises an average (mean, median, etc.) value of the first feature determined from the plurality of filtered perturbation waveforms. In a further embodiment, the second feature value comprises a maximum value, minimum value, or distribution of values determined from the plurality of filtered perturbation waveforms.

At the step of determining 1414, an effect associated with the first perturbation is determined based on a comparison of the first feature value and the second feature value.

The first feature value is a quantitative descriptor of the functional response of the engineered tissue under reference conditions. The second feature value is a quantitative descriptor of the functional response of the engineered tissue under perturbation conditions involving the first perturbation. As such, a comparison of the first and second feature values reveals any change in functional response of the engineered tissue, or effect, occurring as a result of the first perturbation.

For example, the first perturbation may correspond to an application of a compound having an unknown physiological effect. A comparison of the first feature value-corresponding to the peak amplitude of a contractile force waveform of the engineered tissue under reference conditions—and the second feature value—corresponding to the peak amplitude of a contractile force waveform of the engineered tissue under perturbation conditions involving an application of the compound—reveals an increase in average peak amplitude. Consequently, it can be inferred that the compound has an effect associated with increasing the contractile force of the engineered tissue during contraction-relaxation cycles. Beneficially, because the feature values are determined from noise filtered waveforms, effects arising due to the difference between the feature values can be more accurately identified leading to improved processing and potentially improved patient outcomes.

### Systems and Methods for Detecting Spontaneous Tissue Contractions.

A functional response waveform comprises a time-series of values corresponding to measurements of a tissue's functional responses over a time period. As such, functional response waveforms encode the change in functional response (e.g., contractile force, displacement, etc.) of a tissue over a set period of time. In some settings, this change is evoked as a result of an external stimulation applied to the tissue. For example, an electrical stimulation may be periodically applied to a tissue, such as an engineered muscle tissue, at a predetermined pacing frequency (e.g., 0.5 Hz, 1 Hz, 2 Hz, etc.). Given such external stimulation, the functional response waveforms will typically exhibit periodic behavior which can be exploited when identifying and extracting single responses for feature extraction and downstream analysis tasks. In the absence of such stimulation, or when a tissue is exhibiting abnormal contractile behavior, such periodicity may not be present within the waveform. Identifying and extracting single responses from such “spontaneous” contractile response waveforms is thus more challenging. The present disclosure describes systems and methods for efficiently and accurately identifying spontaneous contractions within functional response waveforms which may not exhibit periodic behavior. This enables waveforms which encode spontaneous tissue behavior to be processed and analyzed thereby opening the possibility of such waveforms to be used in a range of downstream tasks such as drug discovery and drug development.

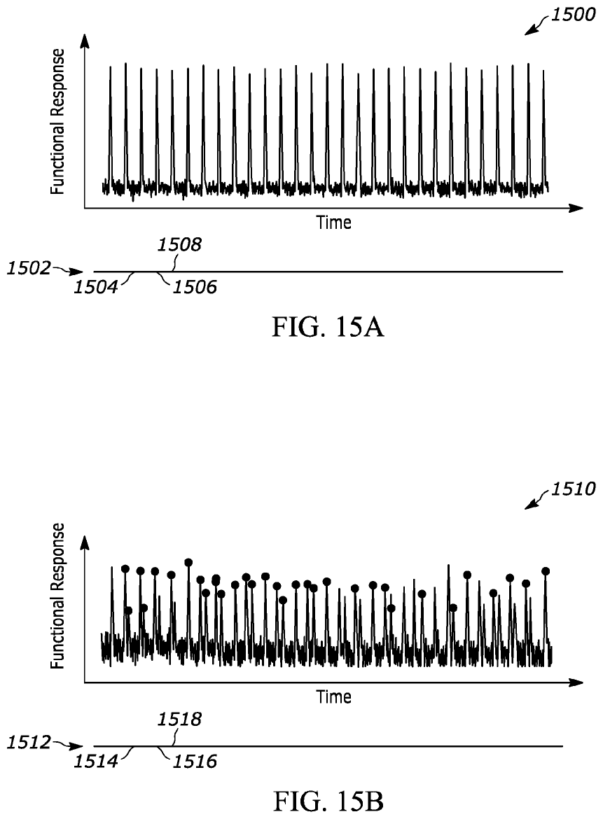

As stated above, in many settings, the contraction-relaxation cycles within a waveform occur due to external stimulation such as an electrical stimulation applied to the tissue at a predetermined pacing frequency (e.g., 1 Hz, 2 Hz, etc.). The regularity of the functional response allows the individual contraction-relaxation cycles to be identified and processed either through identification of repeating patterns or through the incorporation of a priori knowledge of the pacing frequency. This is illustrated in FIG. 15A as described below.

FIG. 15A shows the functional response of a tissue exhibiting periodic contractile behavior.

FIG. 15A shows a functional response waveform 1500 and a timeline 1502 of peak locations which include a first point 1504, a second point 1506, and a third point 1508. The functional response waveform 1500 comprises the functional response (e.g., the contractile force) of a tissue over a time period. A single contractile response (a single peak or contraction) is indicated within the functional response waveform 1500 by a peak—i.e., a local maximum within the functional response waveform 1500. The timeline 1502 of peak locations illustrates the periodicity or regularity of these contractile responses since the points within the timeline 1502—which correspond to the location of peaks or contractions within the functional response waveform 1500—are spaced at approximately regular intervals along the timeline 1502. For example, the interval between the contraction associated with the first point 1504 and the contraction associated with the second point 1506 is substantially the same as the interval between the contraction associated with the second point 1506 and the contraction associated with the third point 1508. As such, the periodicity of the contractions within the functional response waveform 1500 indicate that the tissue exhibits periodic, or regular, contractile behavior.

As stated previously, for waveforms which exhibit periodic behavior, the periodicity can be exploited to help downstream tasks such as contractile response extraction (i.e., extracting a single contraction-relaxation cycle from a waveform). For example, the periodicity, once known or learnt, can be used to determine the expected spacing between single contractions thereby providing a prior for identifying the relative locations of the contractile responses. However, some tissues may exhibit spontaneous, as opposed to periodic, contractile behavior, as illustrated in FIG. 15B.

FIG. 15B shows the functional response of a tissue exhibiting spontaneous contractile behavior.

FIG. 15B shows a functional response waveform 1510 and a timeline 1512 of peak locations which include a first point 1514, a second point 1516, and a third point 1518. The functional response waveform 1510 comprises the functional response (e.g., the contractile force) of a tissue over a time period. As in FIG. 15A, a single contractile response (a single peak or contraction) is indicated within the functional response waveform 1510 by a peak. The timeline 1512 of peak locations illustrates the spontaneity (i.e., lack of periodicity or regularity) of these contractile responses since the points within the timeline 1512—which correspond to the location of peaks or contractions—are irregularly spaced along the timeline 1512. For example, the interval between the contraction associated with the first point 1514 and the contraction associated with the second point 1516 is substantially different to the interval between the contraction associated with the second point 1516 and the contraction associated with the third point 1518. As such, the lack of periodicity of the contractions within the functional response waveform 1510 indicate that the tissue exhibits spontaneous contractile behavior. Alternatively, spontaneous contractile behavior exhibited by a tissue may be periodic but is considered spontaneous because the contractile response of the tissue does not occur as a result of a stimulation of the tissue (e.g., by means of electronic stimulation as described above in relation to FIG. 1). In either case, there is no a priori information which can be used to identify and extract individual contractions from the functional response waveform.

The irregularity of the contractile behavior of the tissue seen in FIG. 15B could be due to several factors. For example, in engineered tissue, the irregularity could be due to an external stimulus no longer being applied to the engineered tissue. Alternatively, the irregularity or spontaneity could be due to one or more conditions of the tissue such as conditions induced due to a disease state, a compound or drug applied to the tissue, or the like.

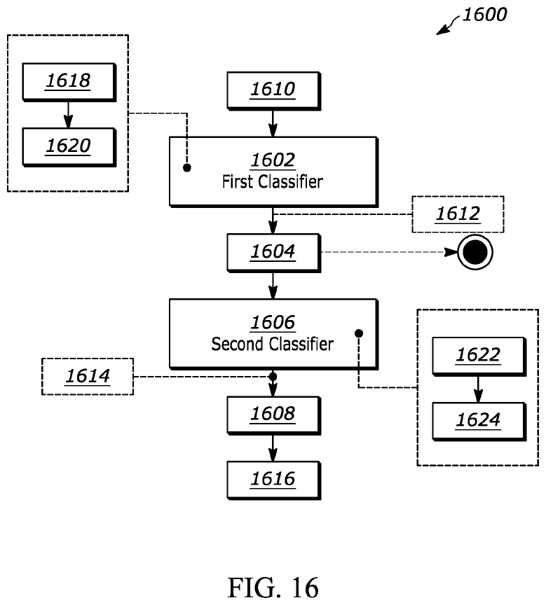

In the absence of stimulation applied to the tissue to evoke contractions, the prior knowledge of the periodicity- or the fundamental characteristics of the regular behavior-cannot be exploited to identify individual contractile responses (i.e., the frequency at which the tissue is stimulated cannot be used to identify individual contraction-relaxation cycles or individual “peaks” within the waveform). According to an aspect of the present disclosure, different approaches may be employed to determine the spontaneous behavior of tissue (such as engineered or artificial tissue) such as that shown in FIG. 16.

FIG. 16 shows a system 1600 for determining the spontaneous behavior of engineered tissue according to an aspect of the present disclosure.

The system 1600 comprises a periodicity classifier 1602, a decisioning unit 1604, a spontaneous contraction classifier 1606, and a profile generator 1608. A waveform 1610 comprising a functional response of an engineered tissue over a time period is provided as input to the periodicity classifier 1602. The periodicity classifier 1602 generates a classification score 1612 based on the waveform 1610. The decisioning unit 1604 determines if the classification score 1612 is indicative of any periodic contractions being present within the waveform 1610. When the classification score 1612 is indicative of no periodic contractions being present within the waveform 1610, the waveform 1610 is provided as input to the spontaneous contraction classifier 1606. The spontaneous contraction classifier 1606 generates a classification score 1614 based on the waveform 1610. The profile generator 1608 generates a behavior profile 1616 for the engineered tissue based on the classification score 1614. In one embodiment, the periodicity classifier 1602 comprises a spectral transformation process 1618 and a prediction model 1620. In one embodiment, the spontaneous contraction classifier 1606 comprises a transformation process 1622 and a thresholding operation 1624.

The system 1600 corresponds to a hierarchical approach to determining the spontaneous behavior of an engineered tissue. By determining the spontaneous behavior of an engineered tissue—as encoded within a functional response waveform—the behavior and contractile location data can be used to perform a number of downstream analysis tasks (e.g., feature extraction, assay development, etc.). A hierarchy of classifiers (i.e., the periodicity classifier 1602 and the spontaneous contraction classifier 1606) are used to identify the global contractile behavior of the engineered tissue and subsequently the local contractile behavior of the engineered tissue. By employing a hierarchy of classifiers, more efficient use of computing resources is made because the global classifier (i.e., the periodicity classifier 1602) initially filters out waveforms which are known to exhibit periodic, or regular, behavior. Consequently, only waveforms which are predicted not to have periodic, or regular, behavior are fed to the local classifier (i.e., the spontaneous classifier 1606). Moreover, this means that the local classifier (i.e., the spontaneous classifier 1606) can be tailored to waveforms which may exhibit spontaneous behavior thereby providing improved identification of the spontaneous behavior (and the locations of spontaneous contractions).

The waveform 1610 comprises a functional response of an engineered, or artificial, tissue over a time period (e.g., over 10 seconds, 20 seconds, 30 seconds, etc.). As stated above in relation to FIG. 1, the functional response can comprise a change in contractile force of the engineered tissue over the time period, or a displacement, or contractile displacement, of the engineered tissue over the time period. Although not shown in FIG. 16, the waveform 1610 is obtained, either directly or indirectly, from a bioreactor such as the bioreactor 102 shown in FIG. 1. In one embodiment, the engineered tissue comprises an artificial muscle tissue such as an artificial cardiac or skeletal muscle tissue.

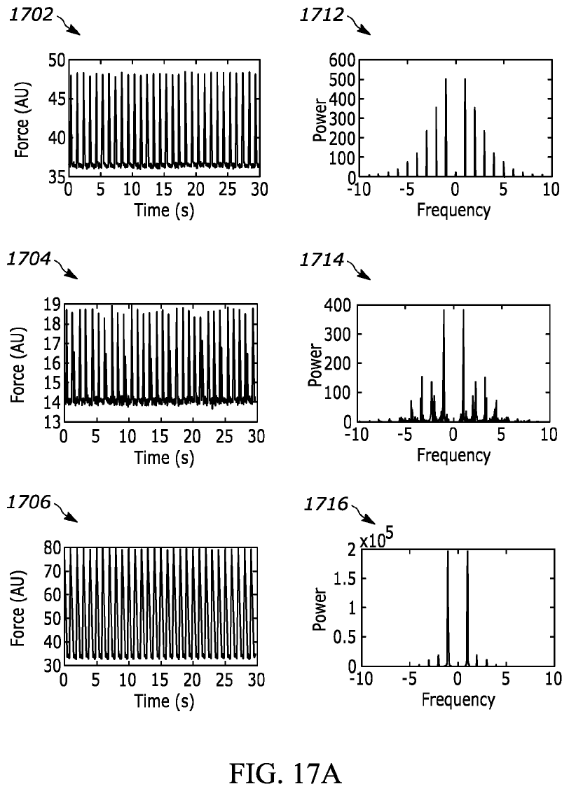

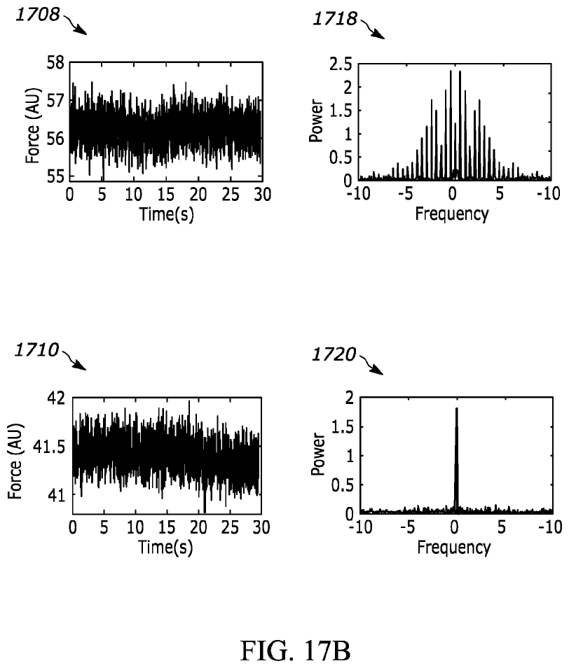

The periodicity classifier 1602 (alternatively referred to as the first classifier, frequency-based global classifier, or global classifier) is used to predict a global characteristic of the waveform 1610. Particularly, the periodicity classifier 1602 is configured to determine whether the waveform 1610 comprises any periodic contractions such as those described in relation to FIG. 15A above. The periodicity classifier 1602 comprises any suitable prediction model which is able to predict, from a time-series input, whether the time-series input contains regular, or periodic, responses (peaks or contractions). In one embodiment, the periodicity classifier 1602 comprises the spectral transformation process 1618 and the prediction model 1620. The spectral transformation process 1618 applies a spectral transformation, such as a Fourier transform or the like, to the waveform 1610 to generate a spectral waveform. The spectral waveform comprises a frequency-based representation of the waveform 1610. The spectral waveform is then used by the prediction model 1620 to determine the classification score 1612 (i.e., the presence or absence of any regular, periodic, contractions within the waveform 1610). Beneficially, the spectral waveform encodes important features of the contractile behavior of a tissue which help improve the classification performance of the prediction model 1620. This is illustrated by the example spectral responses shown in FIGS. 17A and 17B.

FIGS. 17A and 17B show spectral responses of different contractile responses according to embodiments of the present disclosure.

The spectral responses are obtained from the functional response waveforms using a spectral transformation process involving a Fourier transform. Each waveform is associated with an engineered or artificial tissue which exhibits a different contractile response.

The first functional response waveform 1702 is obtained from an engineered tissue exhibiting normal contractile behavior. That is, the contractile response encoded in the first spectral response 1702 follows a periodic (regular) pattern of contraction-relaxation cycles. As can be seen, the power is strongest at 1 Hz (i.e., the pacing frequency) and diminishes with increasing harmonics. The second functional response waveform 1704 is obtained from an engineered tissue exhibiting an abnormal contractile response. That is, some double contractions (or double beats) are present. This is seen in the change in harmonic power in the second spectral response 1714. The third functional response waveform 1706 is obtained from an engineered tissue exhibiting a contractile response having an increased force. That is, the power in the third spectral response 1716 shifts to the primary frequency (1 Hz) from the harmonics. The fourth functional response waveform 1708 is obtained from a tissue exhibiting a spectral response having a decreased contractile force. That is, the power in the fourth spectral response 1708 shifts away from the primary frequency (1 Hz) to the harmonics (1718). The fifth functional response waveform 1710 is obtained from a tissue exhibiting no periodic, or regular, contractile response. That is, the power in the fifth spectral response 1710 is strongest at 0 Hz (1720).

As such, the use of spectral waveforms provide a compact and descriptive representation of the contractile behavior of a tissue which can help improve the discriminative performance of a classifier, or prediction model, tasked with identifying different contractile behaviors.

Referring once again to FIG. 16, the prediction model 1620 uses the spectral waveform (i.e., the frequency-based representation of the waveform 1610) to determine the classification score 1612. The classification score 1612 represents the contractile behavior of the tissue associated with the waveform 1610 and indicates whether the waveform 1610 contains regular, or periodic, contractions. As such, the classification score 1612 is a binary classification which takes one score or value (e.g., “+1”) to indicate that the waveform 1610 comprises periodic contractions and another score or value (e.g., “0” or “−1”) to indicate that the waveform 1610 comprises no periodic contractions.

Alternatively, the prediction model 1620 is a multi-class classifier which predicts a contractile behavior type from the spectral waveform. For example, the prediction model 1620 can be trained to assign a spectral waveform to one of the contractile response types illustrated in FIGS. 17A and 17B (e.g., a classification score of “0” to indicate no periodic contractions, a classification score of “1” to indicate normal periodic contractions, a classification score of “2” to indicate abnormal contractile behavior, etc.).

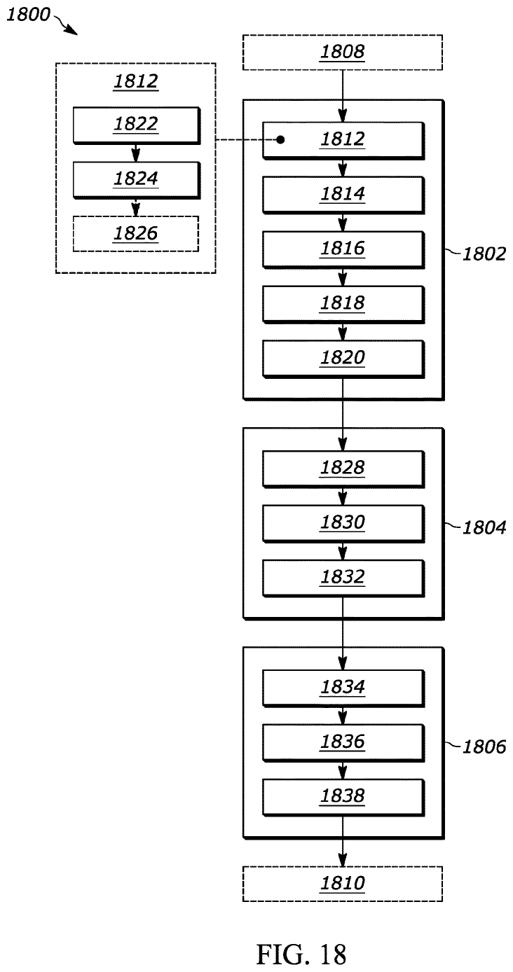

The prediction model 1620 is a trained machine learning model such as a trained neural network, support vector machine, Random Forest, or the like. In one embodiment, the prediction model 1620 is a convolutional neural network such as that shown in FIG. 18.

FIG. 18 shows a convolutional neural network 1800 for predicting contractile behavior according to an embodiment of the present disclosure.

The neural network 1800 comprises a convolutional network 1802, a long short-term memory (LSTM) network 1804, and a fully connected network 1806. An input vector 1808 is received by the convolutional network 1802 and an output vector 1810 is produced by the fully connected network 1806. The convolutional network 1802 comprises a first block 1812, a second block 1814, a third block 1816, a fourth block 1818, and a fifth block 1820. Each block comprises a 2-dimensional (2D) convolution layer, a batch normalization layer, and optionally comprises a rectified linear unit layer. This is illustrated in FIG. 18 by the 2D convolution layer 1822, the batch normalization layer 1824 and the rectified linear unit layer 1826 of the first block 1812. The LSTM network 1804 comprises a flattening layer 1828, a bidirectional LSTM layer 1830, and a dropout layer 1832. The fully connected network 1806 comprises a fully connected layer 1834, a dropout layer 1836, and a softmax layer 1838.

The input vector 1808 comprises a sequence of values corresponding to a spectral waveform (such as the spectral waveforms shown in FIGS. 17A and 17B).

The convolutional network 1802 comprises a sequence of blocks having similar architecture. The first block 1812 comprises the 2D convolution layer 1822 having 4 filters of size 3, the batch normalization layer 1824 (which normalizes the inputs via re-centering and re-scaling), and the rectified linear unit layer 1826. The second block 1814 and the third block 1816 comprise the same architecture: a 2D convolution layer having 7 filters of size 3, a batch normalization layer, and a rectified linear unit layer. The fourth block 1818 comprises a 2D convolution layer having 16 filters of size 3, a batch normalization layer, and a rectified linear unit layer. The fifth block 1820 comprises a 2D convolution layer having 32 filters of size 3 and a batch normalization layer (i.e., the fifth block 1820 does not include the optional rectified linear unit layer).

The LSTM network 1830 comprises the flattening layer 1828 which collapses the spatial dimensions of the output of the fifth block 1820 into a single dimension, the bidirectional LSTM layer 1830 which comprises 8 hidden units, and the dropout layer 1832 which helps to reduce overfitting by randomly setting inputs to zero with a probability of 0.3.

The fully connected network 1806 comprises the fully connected layer 1834 with an output size set to the number of contractile behaviors to predict (i.e., 2 layers are used when predicting periodic contractions and no periodic contractions), the dropout layer 1836 which randomly sets inputs to zero with a probability of 0.05, and the softmax layer 1838 applies a softmax function to the output of the dropout layer 1836.

The output vector 1810 corresponds to a probability vector having a size equal to the number of contractile behaviors being predicted. The output vector 1810 is therefore used to determine a classification score (i.e., the classification score 412 described in relation to FIG. 16 above). For example, via threshold, identifying the maximum, etc.

In one implementation, the neural network 1800 is trained using a synthetic data set comprising 40,000 training samples and 10,000 validation samples. Each element in the synthetic training data set is created by generating a synthetic waveform comprising either periodic or non-periodic contractions, and then applying a spectral transformation (Fourier transform) to generate a spectral waveform. The synthetic data set comprises 25,000 periodic waveforms and 25,000 non-periodic waveforms. Each waveform is associated with a corresponding label indicating whether the waveform is a periodic or non-periodic. The neural network 1800 is trained using minibatch gradient descent with a batch size of 128 and an ADAM solver. The ADAM solver has an initial learning rate of 1e-3 with early stopping based on validation loss.

Referring once again to FIG. 16, the decisioning unit 1604 uses the classification score 1612 obtained from the periodicity classifier 1602 to determine whether the waveform 1610 contains periodic contractions and thus whether the second classifier (i.e., the spontaneous contraction classifier 1606) should be invoked. The decisioning unit 1604 uses a decisioning rule to determine whether to pass the waveform 1610 to the spontaneous contraction classifier 1606 or perform no further action (as indicated by the black circle in FIG. 16). The decisioning rule determines if the classification score 1612 indicates that no periodic contraction is present within the waveform 1610 (e.g., if the classification score 1612 takes a value, such as “0”, which indicates that the periodic classifier 1602 has classified the waveform 1610 as not comprising any periodic contractions). If no periodic contractions are present, then the waveform 1610 is input to the spontaneous contraction classifier 1606.

The spontaneous contraction classifier 1606 (alternatively referred to as a local classifier, a second classifier, or a peak detector) is used to generate the classification score 1614 which indicates whether the waveform 1610 comprises any spontaneous contractions of the engineered tissue over the time period. It may be that the no contractile response of the tissue is observed within the waveform 1610 (i.e., the tissue performed no evoked or spontaneous contractions). As such, both the classification score 1612 and the classification score 1614 would indicate that no contractions—either evoked or spontaneous—are present within the waveform 1610. Alternatively, the waveform 1610 may comprise no evoked contractions but one or more spontaneous contractions (as illustrated in FIG. 15B). The spontaneous contraction classifier 1606 uses the waveform 1610 to determine which of these two behaviors are exhibited by the tissue within the waveform 1610. As such, the spontaneous contraction classifier 1606 is any suitable prediction model or trained machine learning model such as a trained neural network, support vector machine, Random Forest, or the like.

In one embodiment, the spontaneous contraction classifier 1606 comprises the transformation process 1622 and the thresholding operation 1624. In general, the transformation process 1622 generates a transformed waveform from the waveform 1610 and the thresholding operation 1624 is then applied to the transformed waveform. The transformation process 1622 enhances the peaks within the waveform 1610 whilst simultaneously reducing noise. This helps improve the performance of the thresholding operation 1624 which subsequently identifies any peaks which exceed one or more thresholds. If there are any peaks (threshold exceedances) within the transformed waveform which exceed the one or more thresholds, then the waveform 1610 comprises spontaneous contractions of the engineered tissue; otherwise, if there are no threshold exceedances then the waveform 1610 does not comprise any spontaneous contractions of the engineered tissue.

The transformation process 1622 comprises any suitable signal processing operation which can enhance the peaks within the waveform 1610 such as peak sharpening or peak filtering. In one embodiment, the transformation process 1622 comprises a Pan-Tompkins algorithm. In a further embodiment, the transformation process 1622 comprises a modified Pan-Tompkins algorithm. The Pan-Tompkins algorithm was developed to detect the QRS complexes of electrocardiogram (ECG) signals (i.e., the Q wave, R wave, and S wave). The Pan-Tompkins algorithm comprises a sequence of filters which are applied to a waveform to enhance the frequency content (i.e., peaks) of the waveform whilst also removing noise. In general, the Pan-Tompkins algorithm comprises a noise reduction process and a subsequent enhancement process. The noise reduction process applies a band-pass filter (i.e., a low-pass filter followed by a high-pass filter) to the input waveform. The enhancement process comprises a derivative operation, a squaring filter, and an integration filter. The output of the noise reduction process is provided to the derivative operation which provides slope information. The squaring operation enhances the peaks of the output of the derivative operation and the integration filter applies a moving average to the output of the squaring operation. As will be described in more detail below, the modified Pan-Tompkins algorithm of the present disclosure incorporates a stationary waveform transform into the noise reduction process and replaces the squaring filter with a rectification operation. As such, the modified Pan-Tompkins algorithm generates a transformed waveform with better noise reduction characteristics and improved peak enhancement. This helps improve the identification of spontaneous contractions which in turn helps improve the performance of downstream tasks which utilize such information for tasks such as drug discovery and development.

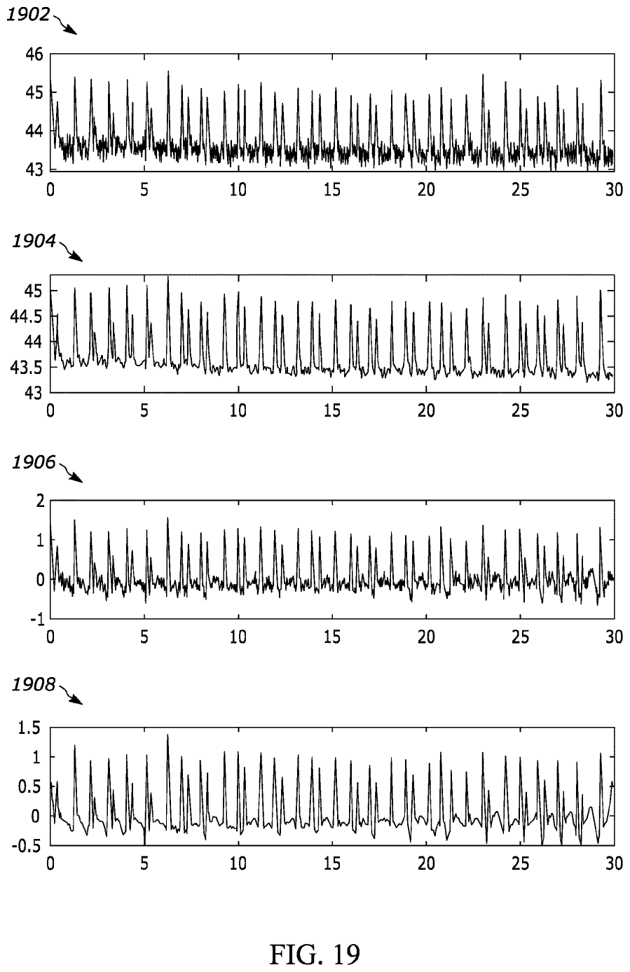

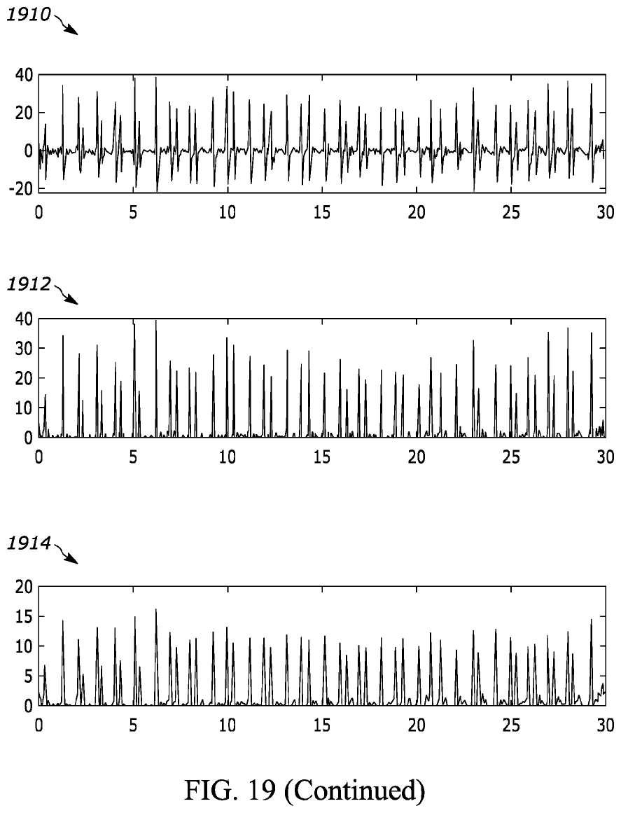

FIG. 19 illustrates the step-wise results of performing a modified Pan-Tompkins algorithm according to an embodiment of the present disclosure.

FIG. 19 shows the result of applying the steps of the modified Pan-Tompkins algorithm of the present disclosure to a waveform 1902. FIG. 19 shows a low-pass filtered waveform 1904, a high-pass filtered waveform 1906, a wavelet transformed waveform 1908, a differentiated waveform 1910, a rectified waveform 1912, and an integrated waveform 1914. Although the modified Pan-Tompkins algorithm is described as performing each of the steps described below, the skilled person will appreciate that, in some implementations, steps can be combined and/or omitted. For example, the algorithm can comprise performing a stationary wavelet transformation and a rectification operation; or a stationary wavelet transformation, a differentiation operation, and a rectification operation. In some embodiments, a normalization operation is applied to the output of the modified Pan-Tompkins algorithm to scale the waveform to a consistent range of values along the y-axis.

The waveform 1902 corresponds to the waveform 1610 shown in FIG. 16 and comprises a functional response (e.g., force) of an engineered tissue over a time period. The result of applying the noise reduction process of the modified Pan-Tompkins algorithm is illustrated in FIG. 19 by the low-pass filtered waveform 1904, the high-pass filtered waveform 1906, and the wavelet transformed waveform 1908. The low-pass filtered waveform 1904 corresponds to the result of applying a low-pass filter to the waveform 1902. The low-pass filter passes portions of the waveform 1902 with a frequency lower than a predetermined cutoff frequency and attenuates portions of the waveform above the predetermined cutoff frequency. Thus, the low-pass filter helps removes major wire misfits from the waveform 1902. In one implementation, the low-pass filter comprises a 2-dimensional Gaussian kernel with standard deviation of 2. The high-pass filtered waveform 1906 corresponds to the result of applying a high-pass filter to the low-pass filtered waveform 1904. The high-pass filtered waveform 1906 therefore corresponds to a band-pass filtered waveform since it is the result of both a low-pass and a high-pass filtering of the waveform 1902. The high-pass filter passes portions of the low-pass filtered waveform 1904 with a frequency above a predetermined cutoff frequency and attenuates portions of the waveform below the predetermined cutoff frequency. Thus, the high-pass filter helps perform baseline alignment whilst removing drift. In one implementation, the high-pass filter comprises an elliptical filter with order of 8, ripple of 0.5 dB, attenuation of 40 dB, and edge frequencies of 1 and 20.

The wavelet transformed waveform 1908 is the result of applying a stationary wavelet transform to the high-pass filtered waveform 1906. As stated above, the modified Pan-Tompkins algorithm of the present disclosure performs a stationary wavelet transform as an additional step of the noise reduction process of the Pan-Tompkins algorithm.

Beneficially, the stationary wavelet transform is shift invariant and helps reduce the noise in waveform whilst retaining important transitional features (changes) within the waveform which may be needed for downstream tasks such as feature extraction. The stationary wavelet transform is an extension of a wavelet transform where the wavelet coefficients are not decimated at every stage. In one implementation, the wavelet transform comprises a discrete stationary wavelet transform (1D) using 5 levels of decomposition and a Daubechies 4 (db4) wavelet.

The result of performing the enhancement process of the modified Pan-Tompkins algorithm is shown in FIG. 19 by the differentiated waveform 1910, the rectified waveform 1912, and the integrated waveform 1914. The differentiated waveform 1910 is the result of performing a differentiation operation to the wavelet transformed waveform 1908. The differentiation operation is used to highlight rapid changes (i.e., contractions). The numerical gradient is computed with uniform spacing between points in all directions. In one implementation, the differentiation operation corresponds to a five-point derivative with the transfer function H(z)=(1/8T) (−z²−2z⁻¹+2z¹+z²) where T is the sampling period. In place of a squaring operation, the modified Pan-Tompkins algorithm of the present disclosure applies a rectification operation to the differentiated waveform 1910 to generate the rectified waveform 1912. The rectification operation clips the differentiated waveform 1910 such that any portions of the differentiated waveform 1910 which are negative are removed. The rectification operation therefore enhances dominant peaks within the waveform. The integrated waveform 1914 corresponds to the result of applying a moving window integration operation to the rectified waveform 1912. The moving window integration operation applies a moving average filter (i.e., a sliding window filtering operation) to the rectified waveform 1912. The moving window integration operation therefore removes short-duration artefacts from the rectified waveform 1912. In one implementation, the moving average filter comprises a moving mean filter calculated over a sliding window with length=[0.1×n] where n is the length of the waveform 1610.

As can be seen from the example in FIG. 19, the output of the modified Pan-Tompkins algorithm (the integrated waveform 1914) comprises a noise reduced version of the input waveform (the waveform 1902) with enhanced representations of the contraction-relaxation cycles. By reducing the noise within the waveform and simultaneously enhancing the peaks (i.e., the contraction-relaxation cycles), the identification of spontaneous contractions within the transformed waveform is improved. This in turn helps improve the performance of downstream tasks which utilize such information for tasks such as drug discovery and development.

Referring once again to FIG. 16, the thresholding operation 1624 identifies any peaks, or threshold exceedances, within the transformed waveform produced by the transformation process 1622. The presence of peaks within the transformed waveform indicates that the waveform 1610 comprises spontaneous contractions. That is, the classification score 1614 would indicate that the waveform 1610 comprises one or more spontaneous contractions if a portion of the transformed waveform meets the thresholding criteria defined by the thresholding operation 1624. Similarly, the absence of threshold exceedances within the transformed waveform indicates that the waveform 1610 does not contain any spontaneous contractions (or evoked contractions by virtue of the hierarchical classification system employed by the system 1600). That is, the classification score 1614 would indicate that the waveform 1610 does not comprise any spontaneous contractions if no portion of the transformed waveform meets the thresholding criteria defined by the thresholding operation 1624.

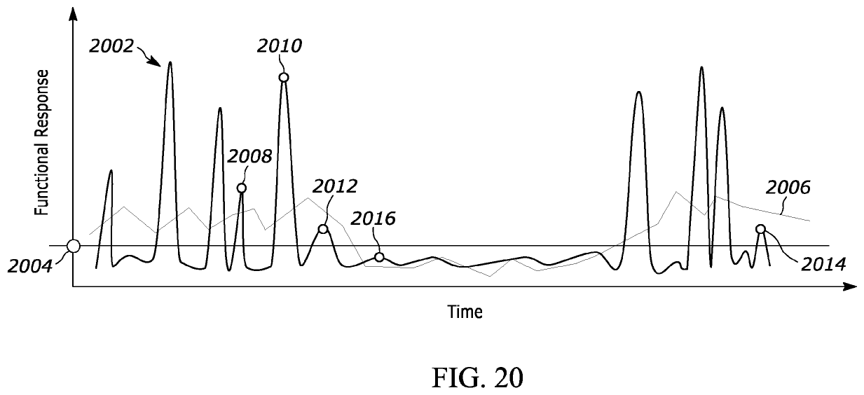

The thresholding operation 1624 involves one or more adaptive thresholds. An adaptive threshold is a threshold based on one or more characteristics, or properties, of the signal (waveform) to which the adaptive threshold is applied. As such, an adaptive threshold will vary depending on the statistical properties of the waveform. In one embodiment, two adaptive thresholds are applied—a uniform adaptive threshold and a dynamic adaptive threshold. The uniform adaptive threshold remains constant over the time period of the waveform 1610 whilst the dynamic adaptive threshold varies over the time period. This is illustrated in FIG. 20.

FIG. 20 illustrates adaptive thresholding of a waveform according to an embodiment of the present disclosure.

FIG. 20 shows a plot of a waveform 2002 (e.g., the transformed representation of the waveform 1610 obtained from the transformation process 1622 shown in FIG. 16) along with a uniform adaptive threshold 2004 and a dynamic adaptive threshold 2006. FIG. 20 further shows a first point 2008, a second point 2010, a third point 2012, a fourth point 2014, and a fifth point 2016, all of which are points on the waveform 2002. The first point 2008 and the second point 2010 exceed both the uniform adaptive threshold 2004 and the dynamic adaptive threshold 2006. The third point 2012 and the fourth point 2014 exceed the uniform adaptive threshold 2004 but not the dynamic adaptive threshold 2006. The fifth point 2016 exceeds the dynamic adaptive threshold 2006 but not the uniform adaptive threshold 2004.

A threshold operation (such as the thresholding operation 1624 shown in FIG. 16) determines one, or both, of the uniform adaptive threshold 2004 and the dynamic adaptive threshold 2006 for the waveform 2002 and utilizes them to identify threshold exceedances (local maxima). These threshold exceedances correspond to spontaneous contractions encoded within the waveform 2002. For example, if the thresholding operation requires both thresholds to be exceeded for a spontaneous contraction to be identified then of the example points highlighted in 20 FIG. 20, the first point 20020 and the second point 2010 on the waveform 2002 would be identified as the location of spontaneous contractions.

The uniform adaptive threshold 2004 is constant over the time period of the waveform 2002 and represents a liberal threshold (i.e., more points within the waveform 2002 will be identified as potential spontaneous contractions than compared to a more conservative threshold). The uniform adaptive threshold 2004 is determined based on a statistical property of the waveform 2002. The statistical property is calculated over the entire time period, or substantially the entire time period, of the waveform 2002. The statistical property includes one or more of: the mean of the waveform 2002; the median of the waveform 2002; and the mean of the maximum and minimum values of the waveform 2002. For example, the uniform adaptive threshold may be set as the mean value of the waveform. In one embodiment, the statistical property is weighted according to a weighting factor α=(0,1]. Thus, the weighting factor is used to control the liberality of the threshold. In one implementation, the weighting factor is chosen according to a manual tuning process whereby a user selects a suitable value for a over a range of waveforms (i.e., a “hold-out set” of waveforms) during a training or calibration phase. The value is then fixed when used in a system such as the system 1600 shown in FIG. 16.

The dynamic adaptive threshold 2006 varies over the time period of the waveform 2002 and represents a conservative threshold (i.e., fewer points within the waveform 2002 will be identified as potential spontaneous contractions than compared to a more liberal threshold). Unlike the uniform adaptive threshold 2004, the dynamic adaptive threshold 2006 is determined over sub-regions of the waveform 2002 such that the dynamic adaptive threshold 2006 varies over the time period. In one embodiment, the dynamic adaptive threshold 2006 is calculated for each time point within the waveform 2002. A window is identified around each time point of the waveform 2002 (e.g., a window containing 3 points, 5 points, 10 points, etc. or a window of 0.5 s, 1 s, 2 s, etc.) and a local threshold for that time point is determined using the portion of the waveform within the window. The local threshold is determined using statistical properties of the portion of the waveform in the same manner as described in relation to the uniform adaptive threshold 2004 (e.g., the mean, median, etc.). As for the uniform adaptive threshold 2004, in one embodiment, the statistical property is weighted according to a weighting factor α=(0,1]. In one implementation, the weighting factor is chosen according to a manual tuning process whereby a user selects a suitable value for a over a range of waveforms (i.e., a “hold-out set” of waveforms) during a training or calibration phase. The value is then fixed when used in a system such as the system 1600 shown in FIG. 16.

Utilizing both the uniform adaptive threshold 2004 and the dynamic adaptive threshold 2006 helps improve the accuracy of the identification of individual spontaneous contractions within the waveform 2002 by reducing the number of false positives (e.g., the fourth point 2014 and the fifth point 2016). This improvement in accuracy can in turn lead to improvements in performance of downstream tasks such as contraction extraction, feature extraction, and uses within drug discovery and development tasks.

Referring once again to FIG. 16, based on the presence or absence of any spontaneous contractions within a waveform, spontaneous contraction classifier 1606 determines the classification score 1614. For example, the classification score may be a binary value indicating the presence (e.g., +1) or absence (e.g., −1) of any spontaneous contractions within the waveform 1610. Alternatively, the classification score may be a vector of spontaneous contraction locations within the waveform 1610 such that an empty vector indicates no spontaneous contractions being identified within the waveform 1610.

The profile generator 1608 generates the behavior profile 1616 for the engineered tissue based on the classification score 1614. In one embodiment, the behavior profile 1616 comprises an indication (e.g., a binary identifier) of whether the waveform 1610 comprises any spontaneous contractions. Additionally, or alternatively, the behavior profile 1616 comprises a summary of the spontaneous contractions within the waveform 1610 such as the number of spontaneous contractions, the average amplitude of the spontaneous contractions, etc. Additionally, or alternatively, the behavior profile 1616 comprises one or more spontaneous contractions (or locations thereof within the waveform 1610) within the waveform 1610. The behavior profile 1616 can then be output for further processing. For example, the behavior profile 1616 can be assigned to the waveform 1610 as a label or as locations of spontaneous contractions in the waveform 1610.

In one embodiment, the behavior profile 1616 is used to extract one or more feature related to the spontaneous contractions from the waveform 1610. For example, features such as those described in relation to FIG. 2 above are extracted around each spontaneous contraction location within the waveform 1610. These features then form a feature vector which serves as a descriptor of the (spontaneous) contractile response of the tissue. This feature vector, or a transformation or summary thereof, can then be used to perform downstream drug discovery and development tasks such as estimating the effect of a compound, or the identification of a disease state.

In another embodiment, the behavior profile 1616 is used as a feature vector to describe the condition or state of the engineered tissue from which the waveform 1610 is generated. That is, the behavior profile 1616, or summary statistics related to the behavior profile—such as the number of spontaneous contractions, average contractile force, etc.—can be used as a phenotype of the engineered tissue. Measuring the changes in a behavior profile (determined using the system 1600) of an engineered tissue when under control conditions and when under perturbed conditions (e.g., treatment conditions involving a drug or compound, disease state, etc.) can be used to identify an effect associated with the perturbed conditions.

The description will now turn to methods for utilizing components, processes, and operations of the above described systems to determine the spontaneous behavior of engineered tissue.

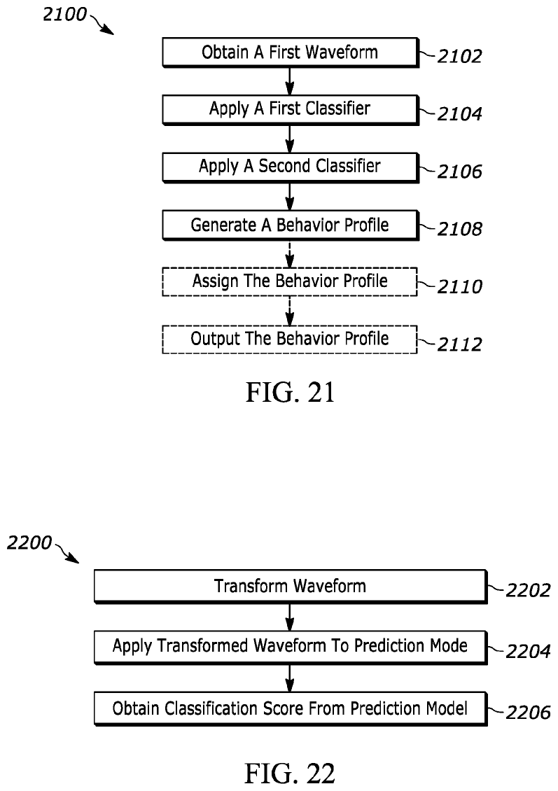

FIG. 21 shows a method 2100 for determining the spontaneous behavior of engineered tissue according to an aspect of the present disclosure.

The method 2100 comprises the steps of obtaining 2102 a first waveform, applying 2104 a first classifier, applying 2106 a second classifier, and generating 2108 a behavior profile. The method 2100 further comprises the optional steps of assigning 2110 the behavior profile and outputting 2112 the behavior profile. In one embodiment, the method 2100 is performed by the signal processing unit 112, or another sub-unit, of the control unit 104 shown in FIG. 1.

At the step of obtaining 2102, a first waveform is obtained (e.g., the waveform 410 shown in FIG. 16). The first waveform comprises a functional response of an engineered tissue over a time period. In one embodiment, the first waveform is obtained directly from a bioreactor within which the engineered tissue is held (e.g., the bioreactor 102 shown in FIG. 1). Alternatively, the first waveform is obtained via an intermediate unit which converts observed measurements obtained from the bioreactor into a functional response waveform. The functional response of the engineered tissue measured within the first waveform is a contractile force, a displacement, a calcium transient response, or the like.

The engineered tissue corresponds to any myopropulsive, or muscle, tissue. The muscle tissue is grown within a device of a bioreactor (e.g., the device 106 of the bioreactor 102 shown in FIG. 1) from cells seeded therein, such as induced pluripotent stem cells (iPSC). In one embodiment, the engineered tissue is an engineered cardiac tissue. More particularly, the engineered tissue may be engineered human cardiac tissue grown from human iPSC-derived cardiomyocytes and ventricular cardiac fibroblasts. Alternatively, the engineered tissue is engineered skeletal muscle tissue.

At the step of applying 2104, a first classifier is applied to the first waveform thereby generating a first classification score (e.g., the periodicity classifier 1602 applied to the waveform 1610 shown in FIG. 16 to generate the classification score 1612). The first classification score is indicative of whether the waveform comprises periodic contractions of the engineered tissue over the time period. As such, the first classifier is alternatively referred to as a periodicity classifier, frequency-based global classifier, or global classifier.

The first classifier predicts a global characteristic of the waveform obtained at the step of obtaining 2102: whether the waveform comprises any periodic (regular or evoked) contractions such as those described in relation to FIG. 15A above. The first classifier comprises any suitable prediction model which is able to predict, from a time-series input, whether the time-series input contains regular, or periodic, responses (peaks or contractions).

The first classifier comprises any suitable prediction model which is able to predict, from a time-series input, whether the time-series input contains regular, or periodic, responses (peaks or contractions). As described in more detail in relation to FIG. 22 below, in one embodiment, the first classifier applied at the step of applying 2104 comprises a spectral transformation process and a prediction model (e.g., the spectral transformation process 1618 and the prediction model 1620 of the periodicity classifier 1602 shown in FIG. 16).

At the step of applying 2106, a second classifier is applied to the waveform to generate a second classification score. The second classifier is applied when the first classification score is indicative of no periodic contractions being present within the waveform (e.g., the spontaneous contraction classifier 1606 is applied to the waveform 1610 shown in FIG. 16 when the classification score 412 indicates that no evoked or periodic contractions are present in the waveform 1610). As such, in one embodiment the method 2100 further comprises, prior to the step of applying 2106, the step of determining whether the waveform comprises one or more periodic contractions based on the classification score obtained at the step of applying 2104. The step of applying 2106 the second classifier is performed if no periodic contractions are identified as being present in the waveform (otherwise, the method 2100 terminates e.g., by returning a suitable indication or notification that the waveform is periodic). The second classification score determined by the second classifier is indicative of whether the waveform comprises spontaneous contractions of the engineered tissue over the time period. The second classifier is alternatively referred to as a spontaneous contraction classifier, a local classifier, or a peak detection.

The second classification score generated by the second classifier indicates whether the waveform obtained at the obtaining 2102 step comprises any spontaneous contractions of the engineered tissue over the time period. It may be that the no contractile response of the tissue is observed within the waveform (i.e., the tissue exhibited no evoked or spontaneous contractions). In such a setting, both the first classification score and the second classification score would indicate that no contractions—either evoked or spontaneous—are present within the waveform. Alternatively, the waveform may comprise no evoked contractions but one or more spontaneous contractions (as illustrated in FIG. 15B). The second classifier uses the waveform obtained at the obtaining 2102 step to determine which of these two behaviors are exhibited by the tissue within the waveform. The second classification score may be a binary value indicating the presence (e.g., +1) or absence (e.g., −1) of any spontaneous contractions within the waveform. Alternatively, the second classification score may be a vector of spontaneous contraction locations within the waveform such that an empty vector indicates no spontaneous contractions being identified within the waveform.

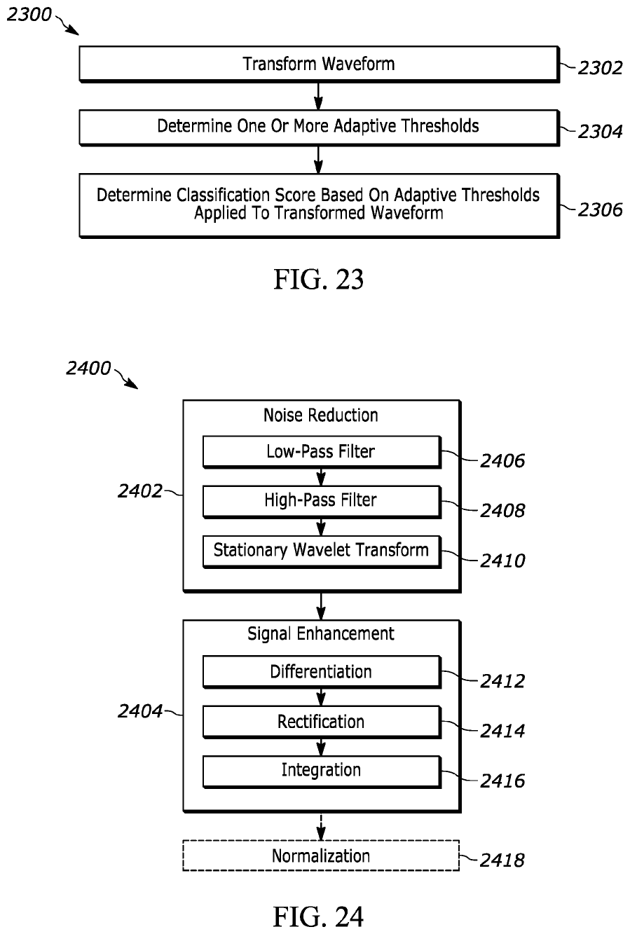

As described in more detail in relation to FIG. 23 below, in one embodiment, the second classifier comprises a transformation process and a thresholding operation (e.g., the transformation process 1622 and the thresholding operation 1624 of the spontaneous contraction classifier 1606 shown in FIG. 16).

At the step of generating 2108, a behavior profile is generated for the engineered tissue during the time period based on the second classification score. In one embodiment, the behavior profile comprises an indication (e.g., a binary identifier) of whether the waveform obtained at the obtaining 2102 step comprises any spontaneous contractions. Additionally, or alternatively, the behavior profile comprises one or more spontaneous contractions associated with one or more portions of the waveform associated with spontaneous contraction locations.

At the optional step of assigning 2110, the behavior profile is assigned to the waveform obtained at the obtaining 2102 step. For example, the behavior profile is assigned as a label for the waveform so that the waveform and the label can be used for further processing or classification (e.g., within a drug discovery or development system). Additionally, or alternatively, when the behavior profile includes the locations of spontaneous contractions within the waveform, the spontaneous contractions can be assigned to the waveform by identifying the location of spontaneous contractions within the waveform (e.g., using metadata, a location vector, or the like).

At the optional step of outputting 2112, the behavior profile is output. In one embodiment, outputting the behavior profile comprises storing, or saving, the behavior profile to a persistent storage such as a non-volatile memory, a non-transitory medium, or the like. Additionally, or alternatively, outputting the behavior profile comprises transmitting the behavior profile via a network (e.g., a local area network, a wide area network, and the like), or displaying the behavior profile for review by a user. In one embodiment, the behavior profile is output in conjunction with the waveform.

FIG. 22 shows a method 2200 for obtaining a classification score from a waveform according to an embodiment of the present disclosure.

In one embodiment, the method 2200 is performed at the step of applying 2104 a first classifier to generate a first classification score in the method 2100. As such, the method 2200 comprises the steps performed by a frequency-based global classifier (periodicity classifier or global classifier) according to one embodiment (e.g., the periodicity classifier 1602 shown in FIG. 16). The method 2200 comprises the steps of transforming 2202 a waveform to generate a transformed waveform, applying 2204 the transformed waveform to a prediction model, and obtaining 2206 a classification score from the prediction model.

At the step of transforming 2202, a spectral transformation process is applied to the waveform (i.e., the waveform obtained at the obtaining 2102 step of the method 2100) to generate a spectral waveform (e.g., the spectral transformation process 418 applied to the waveform 1610 as part of the periodicity classifier 1602 in FIG. 16). The spectral transformation process is any suitable spectral transformation, such as a Fourier transform, which produces a frequency-based representation of the waveform. Example spectral waveforms are shown in FIGS. 17A and 17B.

At the step of applying 2204, the spectral waveform generated at the step of transforming 2202 is input to a prediction model. The prediction model is a trained machine learning model such as a trained neural network, support vector machine, Random Forest, or the like. In one embodiment, the prediction mode is a convolutional neural network such as that shown in FIG. 18 and described in more detail above.

At the step of obtaining 2206, a classification score is obtained from the prediction model based on the spectral waveform. The classification score represents the contractile behavior of the tissue associated with the waveform (i.e., associated with the spectral waveform) and indicates whether the waveform contains regular, or periodic, contractions. In one embodiment, the classification score is a binary classification which takes one score or value (e.g., “+1”) to indicate that the waveform comprises periodic contractions and another score or value (e.g., “0” or “−1”) to indicate that the waveform comprises no periodic contractions.

FIG. 23 shows a method 2300 for obtaining a classification score from a waveform according to an embodiment of the present disclosure.

In one embodiment, the method 2300 is performed at the step of applying 906 the second classifier to generate the second classification score in the method 900. As such, the method 2300 comprises the steps performed by a local classifier (spontaneous contraction classifier or peak detector) according to one embodiment (e.g., the spontaneous contraction classifier 1606 shown in FIG. 16). The method 2300 comprises the steps of transforming 2302 a waveform to generate a transformed waveform, determining 2304 one or more adaptive thresholds, and determining 2306 a classification score based on the one or more adaptive thresholds applied to the transformed waveform.

At the step of transforming 2302, a transformation process is applied to a waveform (i.e., the waveform obtained at the obtaining 2102 step of the method 2100 shown in FIG. 21) to generate a transformed waveform (e.g., the transformation process 1622 applied to the waveform 1610 as part of the spontaneous contraction classifier 1606 shown in FIG. 16). The transformation process comprises any suitable signal processing approach which enhances the peaks within the waveform whilst simultaneously reducing noise. In one embodiment, the transformation process comprises a Pan-Tompkins algorithms. In a further embodiment, the transformation process comprises a modified Pan-Tompkins algorithm as described in more detail in relation to FIG. 24 below.

At the step of determining 2304, one or more adaptive thresholds are determined based on the transformed waveform. As such, values for the one or more adaptive thresholds depend on the transformed waveform. The adaptive thresholds comprise one or more of a uniform adaptive threshold and a dynamic adaptive threshold. The uniform adaptive threshold is constant over the time period of the waveform whilst the dynamic adaptive threshold varies over the time period. Example uniform and dynamic adaptive thresholds shown in FIG. 20 and described above.

At the step of determining 2306, a classification score is determined based on the adaptive thresholds applied to the transformed waveform. As such, a thresholding operation involving the adaptive threshold(s) determined at the step of determining 2304 is performed at the step of determining 2306 (e.g., the thresholding operation 1624 of the spontaneous contraction classifier 1606 shown in FIG. 16). The second classification score determined at the step of determining 2306 is indicative of the waveform comprising spontaneous contractions when one or more portions of the transformed waveform exceed at least one of the one or more adaptive thresholds. Alternatively, the second classification score is indicative of the waveform comprising spontaneous contractions when one or more portions of the transformed waveform exceed both the uniform and the dynamic adaptive threshold. For example, if no portion of the transformed waveform exceeds the adaptive threshold(s) then a classification score of “0” is determined; but if at least one portion of the transformed waveform exceeds the adaptive threshold(s) then a classification score of “+1” is determined (i.e., a classification score indicative of one or more spontaneous contractions being present within the waveform). Alternatively, the classification score comprises a vector of locations within the waveform corresponding to identified spontaneous contraction (such that an empty vector indicates no spontaneous contractions having been identified).

FIG. 24 shows a modified Pans-Tompkins method 2400 according to an embodiment of the present disclosure.

The modified Pans-Tompkins method 2400 comprises a noise reduction process 2402 and a signal enhancement process 2404. The noise reduction process 2402 comprises the steps of filtering 2406 using a low-pass filter, filtering 2408 using a high-pass filter, and transforming 2410 using a stationary wavelet transform. The signal enhancement process 2404 comprises the steps of differentiating 2412, rectifying 2414, and integrating 2416. The modified Pan-Tompkins method 2400 further comprises the optional step of normalizing 2418 the output of the signal enhancement process 2404. In one embodiment, the modified Pan-Tompkins method 2400 is performed at the step of transforming 2302 the waveform in the method 2300.

Although the modified Pan-Tompkins algorithm is described as performing each of the steps described below, the skilled person will appreciate that, in some implementations, steps can be combined and/or omitted. For example, the algorithm can comprise performing a stationary wavelet transformation and a rectification operation; or a stationary wavelet transformation, a differentiation operation, and a rectification operation.

The noise reduction process 2402 of the modified Pan-Tompkins method 2400 generates a noise filtered representation of a waveform. The noise reduction process 2402 comprises applying a band-pass filter to a waveform (e.g., the steps of filtering 2406 and filtering 2408) and, in contrast to the standard Pan-Tompkins algorithm, subsequently transforming 2410 the output of the band-pass filter using a stationary wavelet transform.

At the step of filtering 2406, a low-pass filter is applied to the waveform to generate a low-pass filtered waveform. The low-pass filter helps removes major wire misfits from a waveform. An example low-pass filtered waveform is shown by the low-pass filtered waveform 1904 in FIG. 19 which corresponds to the result of applying a low-pass filter to the waveform 1902. In one implementation, the low-pass filter comprises a 2-dimensional Gaussian kernel with standard deviation of 2.

At the step of filtering 2408, a high-pass filter is applied to the low-pass filtered waveform (generated at the step of filtering 2406) to generate a high-pass filtered waveform. The high-pass filter helps perform baseline alignment whilst removing drift. An example high-pass filtered waveform is shown by the high-pass filtered waveform 1906 in FIG. 19 which corresponds to the result of applying a high-pass filter to the low-pass filtered waveform 1904. In one implementation, the high-pass filter comprises an elliptical filter with order of 8, ripple of 0.5 dB, attenuation of 40 dB, and edge frequencies of 1 and 20.

At the step of transforming 2410, a stationary wavelet transform is applied to the high-pass filtered waveform (generated at the step of filtering 2408) to generate a transformed waveform. The stationary wavelet transform helps improve the denoising performed at the noise reduction process 2402 thereby improving the classification performance of the spontaneous contraction classifier. An example transformed waveform is shown by the wavelet transformed waveform 1908 in FIG. 19 which corresponds to the result of applying a stationary wavelet transform to the high-pass filtered waveform 1906. In one implementation, the wavelet transform comprises a discrete stationary wavelet transform (1D) using 5 levels of decomposition and a Daubechies 4 (db4) wavelet.

The signal enhancement process 2404 of the modified Pan-Tompkins method 2400 generates an enhanced representation of the noise filtered waveform produced by the noise reduction process 2402. In contrast to the standard Pan-Tompkins algorithm, the squaring operation is replaced by the step of rectifying 2414.

At the step of differentiating 2412, the noise reduced waveform obtained from the noise reduction process 2402 is differentiated using a differentiation operation to generate a differentiated waveform. The differentiation operation is used to highlight rapid changes (i.e., contractions). An example of a differentiated waveform is shown by the differentiated waveform 710 in FIG. 19 which corresponds to the result of differentiating the wavelet transformed waveform 1908. In one embodiment, the differentiation operation corresponds to a five-point derivative with the transfer function H(z)=(1/8T) (−z²−2z⁻¹+2z¹+z²) where T is the sampling period.

At the step of rectifying 2414, the differentiated waveform is rectified thereby generating a rectified waveform. That is, at the step of rectifying 2414, the differentiated waveform is clipped such that any portions of the differentiated waveform which are negative are removed. The rectification operation therefore enhances dominant peaks within the waveform. An example of a rectified waveform is shown by the rectified waveform 1912 in FIG. 19 which corresponds to the result of rectifying the differentiated waveform 1910.

At the step of integrating 2416, a moving window integration operation is applied to the rectified waveform to generate an integrated waveform. The moving window integration operation removes short-duration artefacts from the rectified waveform. An example of an integrated waveform is shown by the integrated waveform 1914 in FIG. 19 which corresponds to the result of applying a moving window integration operation to the rectified waveform 1912. In one implementation, the moving average filter comprises a moving mean filter calculated over a sliding window with length=[0.1×n] where n is the length of the waveform 1610.

At the optional step of normalizing 2418, the output of the signal enhancement process 2404 (e.g., the integrated waveform produced at the step of integrating 2416) is normalized to scale the values of the waveform to a set range along the y-axis (e.g., between 0 and 1).

### Systems and Methods for Contraction Type Classification of Engineered Tissue.

The present disclosure presents systems and methods for classifying and processing of functional response waveforms to enable the efficient and effective extraction of features across a range of contraction types.

FIG. 25 illustrates a double contraction type contraction-relaxation cycle model according to an embodiment of the present disclosure.

FIG. 25 shows a single contraction model 2508, as described in detail above in relation to FIG. 3 and a double contraction model 2510 (alternatively referred to as a double contraction type contraction-relaxation cycle model, a double type model, or double model). The double contraction model 2510, f_{D}(t), comprises a combination of two single contraction models fₘ_{₁}(t) and fₘ_{₂}(t). Each of the single contraction models are defined by their own parameter sets, θ₁=[A₁, B, t₀_{₁}, t_{d}_{₁}, k_{c}_{₁}, kᵣ_{₁}] and θ₂=[A₂, B, t₀_{₂}, t_{d}_{₂}, k_{c}_{₂}, kᵣ_{₂}] such that the overall double contraction model 2510 is parameterized by the parameter set θ=[A₁, t₀_{₁}, t_{d}_{₁}, k_{c}_{₁}, kᵣ_{₁}, A₂, t₀_{₂}, t_{d}_{₂}, k_{c}_{₂}, kᵣ_{₂}, B]. In this model, the y-axis shift B is shared across both of the single models, though in some embodiments B is allowed to vary across models. By modelling functional responses having a double contraction type using the double contraction model 2510, the individual components of the contractile response can be modelled and thus vary independently. This allows double contraction type functional responses to be modelled efficiently and effectively thus improving the performance of downstream tasks which utilize such models.

FIG. 26 shows a system 2600 for processing of functional response waveforms having different contraction types according to an embodiment of the present disclosure.

The system 2600 comprises a contraction type classifier 2602, a single contraction model 2604, a double contraction model 2606, and a waveform generator 2608. The system 2600 receives an input waveform 2610 and produces an output waveform 2612 which comprises a noise filtered representation of the input waveform 2610. The input waveform 2610 comprises at least one contraction response and at least one relaxation response of an engineered tissue. The input waveform 2610 has a length corresponding to an expected contraction-relaxation cycle of an engineered tissue and comprises either a single contraction of the engineered tissue (i.e., the input waveform is a single contraction type) or a double contraction of the engineered tissue (i.e., the input waveform is a double contraction type). To identify the relevant contraction type, the input waveform 2610 is classified by the contraction type classifier 2602 into a predicted contraction type 2614 (alternatively referred to as a predicted contraction-relaxation cycle type, predicted type, or predicted cycle type). Based on the predicted contraction type 2614, either the single contraction model 2604 or the double contraction model 2606 is fit to the input waveform 2610 resulting in either a fit single model 2616 or a fit double model 2618. The waveform generator 2608 utilizes the fit model (i.e., the fit single model 2616 or the fit double model 2618) to generate an output waveform 2612 which comprises a noise filtered representation of the input waveform 2610. For example, the waveform generator 2608 samples a number of points (e.g., 100, 200, 500, 1000, etc.)

from the fit model (i.e., the fit single model 2616 or the fit double model 2618) over the time period of the input waveform 2610 to generate the output waveform 2612. Beneficially, this allows high-resolution waveform data to be generated which helps ensure that more accurate features of the functional response of the engineered tissue are extracted. This in turn helps improve the accuracy and efficacy of downstream tasks involving the functional response features.

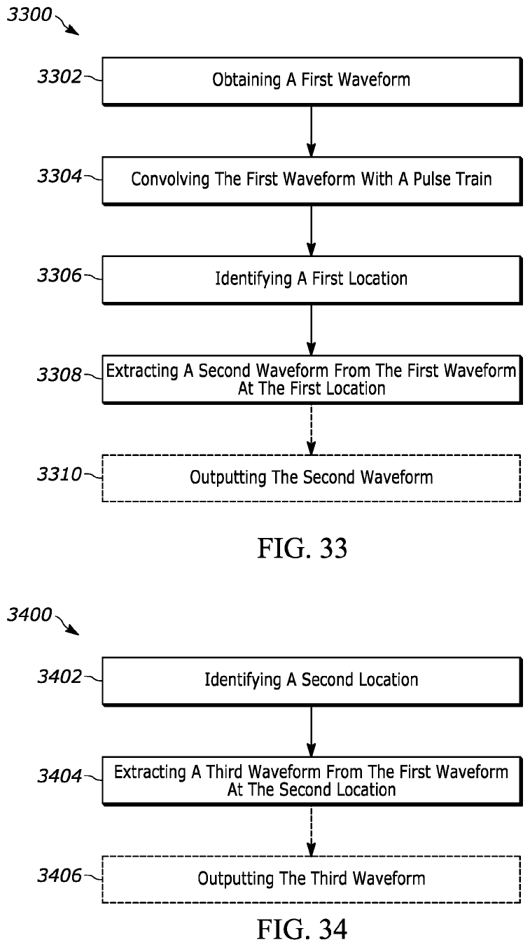

In one embodiment, the input waveform 2610 is extracted from a larger waveform of functional responses using a method such as that described in relation to FIG. 33 below.

The fit single model 2616 and the fit double model 2618 comprise parameter values for the relevant contraction-relaxation cycle model. In one embodiment, the parameter values in the fit single model 2616 or the fit double model 2618 are further refined using an optimization approach. As will be described in more detail below in relation to FIG. 29, the optimization approach utilized comprises a simplex search algorithm such as the Nelder-Mead method.

In one embodiment, one or more feature values are extracted from the output waveform 2612 and the one or more feature values are outputted. Examples of the one or more feature values that are extracted from the output waveform 2612 include one or more of a twitch or peak amplitude value (i.e., the peak amplitude 206 shown in FIG. 2), a contraction time value (i.e., the time to peak amplitude 208 shown in FIG. 2), a maximum contraction slope value (i.e., the maximum rate of development 214 shown in FIG. 2), a relaxation time value (i.e., the time to peak decline 210 shown in FIG. 2), a maximum relaxation slope value (i.e., the maximum rate of declination 216), and a twitch duration value (i.e., the duration 212). For waveforms corresponding to a double contraction type, the features can comprise two values for each feature extraction. For example, a first peak amplitude value associated with the first peak within the double contraction type waveform and a second peak amplitude value associated with the second peak within the double contraction type waveform.

The single or double contraction-relaxation model can be fit using any suitable parameter fitting technique such as least squares based fitting, or machine learning based fitting such as that shown in FIG. 6.

FIG. 27 shows contraction type classifications for three waveforms according to an embodiment of the present disclosure.

FIG. 27 shows a baseline waveform 2702, a first perturbed waveform 2704, and a second perturbed waveform 2706. The waveforms comprise functional responses (contractile force) of an artificial tissue under baseline conditions and two perturbed conditions. Examples of perturbed conditions are treatment of a tissue with a drug or compound, different cell lines, different disease states, physical perturbation of the artificial tissue, changes made to the platform or bioreactor containing the artificial tissue, and the like. The baseline waveform 2702, the first perturbed waveform 2704, and the second perturbed waveform 2706 are associated with a baseline classification vector 2708, a first perturbed classification vector 2710, and a second perturbed classification vector 2712 respectively. The baseline classification vector 2708 comprises a plurality of classification values associated with functional response waveforms within the baseline waveform 2702, such as the first classification value 2714 which comprises a contraction type classification for an associated functional response waveform 2716 within the baseline waveform 2702. The first perturbed classification vector 2710 comprises a plurality of classification values associated with functional response waveforms within the first perturbed waveform 2704, such as the second classification value 2718 and the third classification value 2720. The second classification value 2718 and the third classification value 2720 comprise contraction type classifications for associated functional response waveforms 2722, 2724 within the first perturbed waveform 2704. The second perturbed classification vector 2712 comprises a plurality of classification values associated with functional response waveforms within the second perturbed waveform 2706.

The classification vectors (e.g., the baseline classification vector 2708) are obtained by extracting a plurality of functional response waveforms from each waveform (as described in more detail below in relation to FIG. 33) and then determining a contraction type for each functional response waveform using a prediction model (e.g., the prediction model 600 described above in relation to FIGS. 6 and 7). The contraction types are either a single contraction type or a double contraction type. As shown in FIG. 27, the first classification value 2714 and the second classification value 2718 are single contraction types (as indicated by the light gray shading) because the associated functional response waveforms 2716, 2722 are determined by the prediction model to contain single contractions. In contrast, the third classification value 2720 is a double contraction type (as indicated by the dark gray shading) because the associated functional response waveform 2724 is determined by the prediction model to contain a double contraction.

As stated above, the waveforms comprise functional responses of an artificial tissue under baseline conditions and two perturbed conditions. As such, any changes in the distribution of contraction types within the classification vectors indicate a change in behavior of the artificial tissue occurring as a result of a perturbation of the artificial tissue. In the example shown in FIG. 27, the baseline waveform 2702 comprises the functional responses of the artificial tissue under reference, or control, conditions, the first perturbed waveform 2704 comprises the functional responses of the artificial tissue under a first dose of isoproterenol, and the second perturbed waveform 2706 comprises the functional responses of the artificial tissue under a second dose of isoproterenol. As can be seen by comparing the first perturbed classification vector 2710 and the second perturbed classification vector 2712 to the baseline classification vector 2708 the contractile behavior of the artificial tissue changes under increasing doses of isoproterenol. As shown most clearly in the second perturbed classification vector 2712, isoproterenol induces regular double contractions of the artificial tissue which are not present in the behavior of the artificial tissue under control conditions.

The description will now turn to methods for utilizing the above described systems and models for classifying and processing of functional response waveforms.

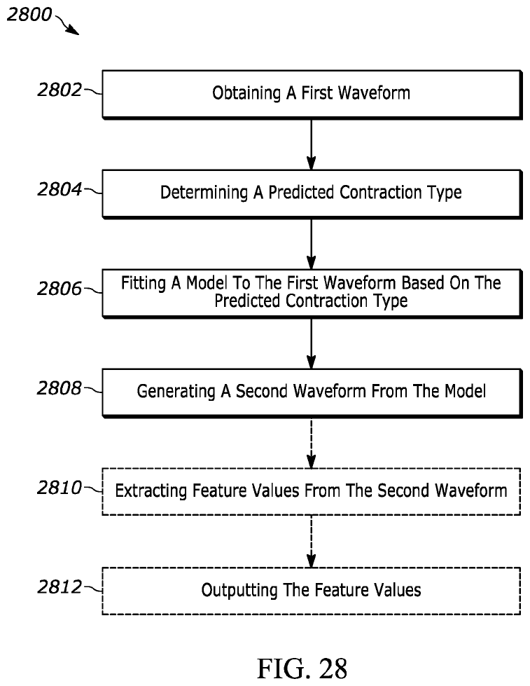

FIG. 28 shows a method 2800 for processing a functional response waveform according to an aspect of the present disclosure.

The method 2800 comprises the steps of obtaining 2802 a first waveform, determining 2804 a predicted contraction type, fitting 2806 a model to the first waveform based on the predicted contraction type, and generating 2808 a second waveform from the model. The method 2800 further comprises the optional steps of extracting 2810 feature values from the second waveform and outputting 2812 the feature values. In one embodiment, the method 2800 is performed by the model fitting unit 104-1 of the control unit 104 shown in FIG. 1.

In general, the method 2800 is used to generate a noise filtered, or noise suppressed, representation of a contraction-relaxation cycle waveform (functional response waveform) which may comprise either a single contraction type response or a double contraction type response. In embodiments, the contraction-relaxation cycle waveform is obtained from hardware such as a bioreactor (e.g., the bioreactor 102 shown in FIG. 1) which may introduce noise into the waveform due to factors such as sensor variability, signal transmission, signal conversion, and the like. The contraction-relaxation cycle waveform thus comprises a potentially noisy representation of the functional response of an engineered tissue during a single contraction-relaxation cycle. Beneficially, the method 2800 efficiently classifies the contraction type of the waveform and efficiently generates a noise filtered representation of the contraction-relaxation cycle thereby improving the accuracy of the extracted features which in turn helps improve the performance of downstream tasks which utilize such features.

At the step of obtaining 2802, a first waveform is obtained. The first waveform comprises at least one contraction response and at least one relaxation response of an engineered, or artificial, tissue. The first waveform has a predetermined length corresponding to an expected length of a contraction-relaxation cycle of the engineered tissue.

The first waveform (or first functional response waveform) captures the functional response of the artificial tissue. The functional response is a contractile force of the artificial tissue (e.g., a contractile force as measured using data obtained from a sensor assembly 108 of a bioreactor 102 within which the artificial tissue is grown or sustained). Alternatively, the functional response is a contractile displacement, a transient calcium response, or a change in membrane potential.

The artificial tissue, or engineered tissue, comprises engineered muscle tissue such as engineered cardiac tissue or engineered skeletal muscle tissue. In one embodiment, the first waveform is obtained from bioreactor (e.g., the bioreactor 102 shown in FIG. 1) containing the artificial tissue. As such, the first waveform is obtained from a waveform, obtained from the bioreactor, which comprises a plurality of single or double contraction-relaxation cycles of the artificial tissue. In one embodiment, the first waveform is obtained, or extracted, from the waveform using an extraction method such as method 3300 described in more detailed below in relation to FIG. 33.

The predetermined length of the first waveform, which corresponds to the expected length of the contraction-relaxation cycle, is from 0.05 s to 10 s and more particularly is from 0.15 s to 1 s. In one embodiment, the predetermined length is proportional to a frequency at which the engineered tissue is stimulated when the first waveform is recorded. For example, if the engineered tissue is stimulated at 1 Hz then the predetermined length is 1 s because the expected length of a contraction-relaxation cycle of the engineered tissue will be 1 s.

At the step of determining 2804, a predicted contraction type of a plurality of contraction types is determined for the first waveform. The plurality of contraction types includes a single contraction type and a double contraction type.

The predicted contraction type is determined by a classifier based on the first waveform input to the classifier. As described in more detail above in relation to FIGS. 6 and 7A-7D, the classifier comprises a trained machine learning model such as a trained neural network. In one embodiment, the classifier comprises a convolutional neural network, a dilated convolutional neural network, and a long short-term memory network. Further architectural details of the classifier are given in relation to FIGS. 6 and 7A-7D above.

At the step of fitting 2806, a model is fit to the first waveform based on the predicted contraction type. The model parameterizes growth of the at least one contraction response of the engineered tissue independently of growth of the at least one relaxation response of the engineered tissue. As such, the model does not assume that the underlying functional response is symmetrical. This allows the model to be fit efficiently and accurately to a range of functional responses from a large variety of engineered tissue types. More efficient and accurate model fitting helps to generate more accurate features which in turn leads to better data being generated.

The model fit to the first waveform is chosen based on the predicted contraction type. If the predicted contraction type is a single contraction type, then a single contraction type model is fit (as illustrated and described in relation to FIG. 3 above). If the predicted contraction type is a double contraction type, then a double contraction type model is fit (as illustrated and described in relation to FIG. 25 above).

The single contraction type model comprises a first contraction function, f_{c}(t), and a first relaxation function, fᵣ(t). The double contraction type model comprises a first contraction function and a first relaxation function as well as a second contraction function and a second relaxation function. In one embodiment, the contraction functions are rising logistic functions having a positive growth rate and the relaxation functions are falling logistic functions having a negative growth rate. The contraction responses of the single or double contraction-relaxation cycle are thus modeled by the rising logistic functions and the relaxation responses of the single or double contraction-relaxation cycle is modeled by the falling logistic functions. The single contraction type model comprises a product of the first rising logistic function and the first falling logistic function. The double contraction type model comprises the combination (i.e., additive combination) of two single contraction type models.

The contraction-relaxation cycle models comprise a plurality of parameters associated with the at least one contraction response and the at least one relaxation response. The plurality of parameters comprises at least on maximum value parameter, A, at least one rate of rise parameter, k_{c}, at least one rate of fall parameter, kᵣ, a y-shift parameter, B, at least one rising x-shift parameter, t₀, and at least one falling x-shift parameter, t_{d}. The relation of each of these parameters to the overall single or double contraction-relaxation cycle models are shown and described in more detail in relation to FIGS. 3 and 25 above.

In one embodiment, the model is fit to the first waveform using a prediction of parameter values obtained from a machine learning model (e.g., the prediction model 600 shown in FIG. 6), as described in more detail in relation to the method 2900 of FIG. 29 below.

At the step of generating 2808, a second waveform is generated from the model fit to the first waveform such that the second waveform comprises a noise filtered representation of the first waveform. For example, a number of points (e.g., 100, 200, 500, 1000, etc.) are sampled from the model fit at the fitting 2806 step over the time period of the first waveform. Beneficially, this allows high-resolution waveform data to be generated from the contraction-relaxation cycle which helps ensure that more accurate features of the functional response of the engineered tissue are extracted. This in turn helps improve the accuracy and efficacy of downstream tasks involving the functional response features.

Optionally, the second waveform may be output. In one embodiment, outputting the second waveform comprises storing, or saving, the second waveform to a persistent storage such as a non-volatile memory, a non-transitory medium, or the like. Additionally, or alternatively, outputting the second waveform comprises transmitting the second waveform via a network (e.g., a local area network, a wide area network, and the like), or displaying the waveform for review by a user.

At the optional step of extracting 2810, one or more feature values are extracted from the second waveform.

The second waveform is a noise filtered representation of the first waveform (i.e., a noise filtered representation of the single or double contraction-relaxation cycle) and, as such, enables more accurate values of features of the underlying functional response to be extracted. The one or more feature values include one or more of at least one contraction-relaxation cycle, or peak, amplitude value (i.e., the peak amplitude 206 shown in FIG. 2), a contraction time value (i.e., the time to peak amplitude 208 shown in FIG. 2), a maximum contraction slope value (i.e., the maximum rate of development 214 shown in FIG. 2), a relaxation time value (i.e., the time to peak decline 210 shown in FIG. 2), a maximum relaxation slope value (i.e., the maximum rate of declination 216 shown in FIG. 2), and a contraction-relaxation cycle duration value (i.e., the duration 212 shown in FIG. 2).

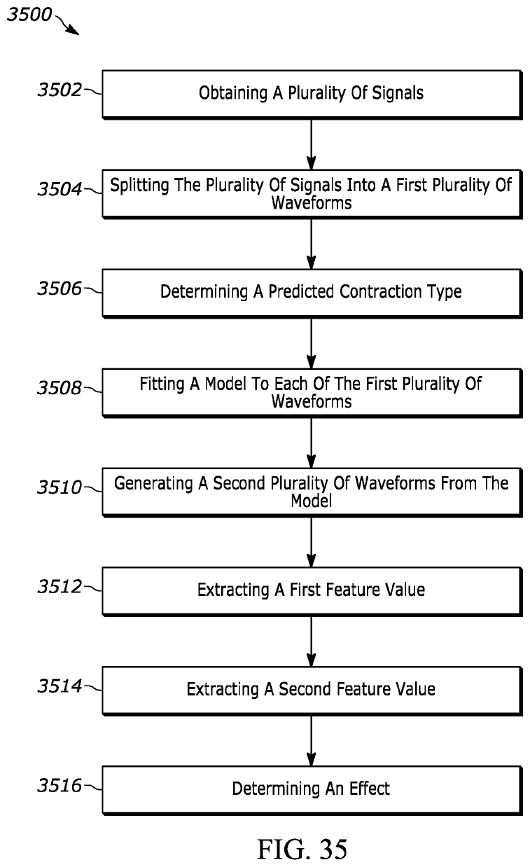

Once extracted, the one or more feature values may be used as a quantitative descriptor of the single or double contraction-relaxation cycle and thus provide a numeric representation of the functional response of the artificial tissue. As will be described in more detail in relation to FIG. 35, such features are used in various downstream processing tasks such as effect identification in drug discovery and development. At the optional step of outputting 2812, the one or more feature values extracted from the second waveform are output. In one embodiment, outputting the one or more feature values comprises storing, or saving, the one or more feature values to a persistent storage such as a non-volatile memory, a non-transitory medium, or the like. Additionally, or alternatively, outputting the one or more feature values comprises transmitting the one or more feature values via a network (e.g., a local area network, a wide area network, and the like), or displaying the one or more feature values for review by a user.

FIG. 29 shows a method 2900 for fitting a model to a functional response waveform according to an embodiment of the present disclosure.

In one embodiment, the method 2900 is performed as part of the fitting 906 step of the method 900. The method 2900 comprises the step of predicting 2902 a plurality of values for the plurality of parameters and further comprises the optional step of optimizing 2904 the plurality of values.

The steps in the method 2900 are used to predict parameter values for the model from the first waveform. The model is either a single contraction type model (FIG. 3) or a double contraction type model (FIG. 25). Which model is fit by the method 2900 is dependent on the predicted contraction type determined at the determining 2804 step of the method 2800. A trained machine learning model (e.g., the parameter estimation model shown in FIG. 6) is used to predict the parameter values such that the fit model closely approximates the first waveform.

At the step of predicting 2902, a plurality of values are predicted for the plurality of parameters of the model such that the model fit to the first waveform comprises the plurality of values for the plurality of parameters. The plurality of values are predicted by either a single contraction type model or a double contraction type model.

At the optional step of optimizing 2904, the plurality of values determined at the predicting 2902 step are optimized by minimizing an error between the first waveform and the model fit to the first waveform (i.e., using the plurality of values for the plurality of parameters of the model).

Given a plurality of values for the model, θ̅, determined at the step of predicting 2902, an updated plurality of values, θ̅′, are sought at the step of optimizing 2904 such that L_{θ̅′}<L_{θ̅}. Here, L_{θ}(⋅) is a cost, or loss, function which measures the error between the first waveform, X₁, and the model, f_{θ}, fit to the first waveform according to the set of parameter values θ. A lower value of L indicates a better fit of the model to the first waveform. In one embodiment, the cost function, L, is the root mean square error:

$${L}_{θ} = \sqrt{𝔼 ( {( {f}_{θ} - {X}_{1} )}^{2} )} . \qquad ( 5 )$$

Which in one embodiment corresponds to:

$${L}_{θ} = \sqrt{\frac{1}{N} ( {\sum }_{i = 1}^{N} {( {f}_{θ} ( i ) - {x}_{i} )}^{2} )} . \qquad ( 6 )$$

The optimization of θ is a multidimensional problem because the model involves multiple parameters. Minimizing L_{θ} therefore requires the simultaneous fitting of multiple parameters. To perform this minimization, in one embodiment the plurality of values are optimized using a simplex search algorithm such as the Nelder-Mead method. Beneficially, performing optimization after obtaining an initial prediction of the parameters helps obtain an accurate model fit which helps avoid local minima whilst making better use of processing resources because the optimization process starts at a solution which is expected to be close to an optimal solution.

FIG. 30 shows a method 3000 for training a classifier to predict a tissue contraction type from a functional response waveform.

The method 3000 comprises the steps of obtaining 3002 a plurality of waveforms, extracting 3004 a first plurality of parameter sets, extracting 3006 a second plurality of parameter sets, determining 3008 a plurality of parameter set distributions, generating 3010 a synthetic training data set, and training 3012 a classifier on the synthetic training data set. In one embodiment, the method 3000 is performed by the control unit 104, or a sub-unit thereof, shown in FIG. 1.

In many situations, the efficacy of prediction models (e.g., machine learning models such as deep neural networks) are limited by the amount and quality of training data available. In the absence of high-volume, high-quality, training data, prediction models will often fail to produce adequate outputs. In the present disclosure, this problem would lead to inaccurate identification of single or double type contractions which would subsequently reduce the effectiveness and applicability of using such identifiers in practice for tasks such as drug discovery and development. The method 3000 seeks to address such problems by generating high-fidelity synthetic training data thereby allowing an almost limitless amount of data to be generated. This helps to improve the performance of the prediction model being trained which subsequently improves the accuracy of model predictions. This improvement of accuracy helps drive improvements to downstream tasks which utilize classifications obtained from such models.

At the step of obtaining 3002, a plurality of waveforms are obtained. The plurality of waveforms comprise functional responses of a one or more engineered tissues. Each of the plurality of waveforms has a predetermined length corresponding to an expected length of a contraction-relaxation cycle of an engineered tissue.

At the step of extracting 3004, a first plurality of parameter sets is extracted from a first subset of the plurality of waveforms associated with a single contraction type. A first parameter set of the first plurality of parameter sets characterizes a first waveform of the first subset of waveforms.

At the step of extracting 3006, a second plurality of parameter sets are extracted from a second subset of the plurality of waveforms associated with a double contraction type. A second parameter set of the second plurality of parameter sets characterizes a second waveform of the second subset of waveforms.

At the step of determining 3008, a plurality of parameter set distributions are determined. The plurality of parameter set distributions comprise a first parameter set distribution determined from the first plurality of parameter sets and a second parameter set distribution determined from the second plurality of parameter sets.

At the step of generating 3010, a synthetic training data set is generated. Each element of the synthetic training data set comprises a synthetic waveform and a corresponding tissue contraction type associated with the synthetic waveform. The synthetic waveform is generated using a parameter set distribution of the plurality of parameter set distributions associated with the corresponding tissue contraction type.

At the step of training 3012, the classifier is trained using the synthetic training data set. The classifier trained using the synthetic training data set determines a predicted tissue contraction type for an input waveform.

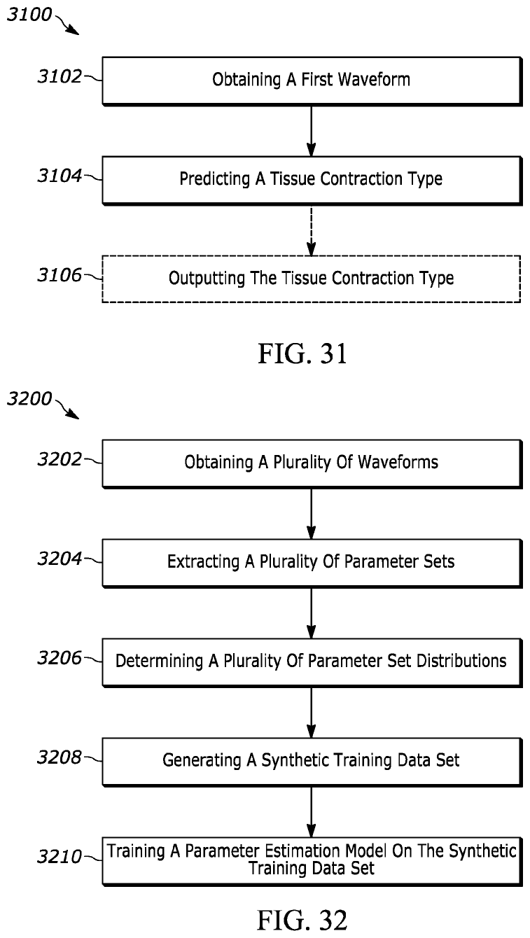

FIG. 31 shows a method 3100 for predicting a tissue contraction type for a waveform using a synthetically trained classifier according to an embodiment of the present disclosure.

The method 3100 comprises the steps of obtaining 3102 a first waveform and predicting 3104 a tissue contraction type. The method 3100 also comprises the optional step of outputting 3106 the tissue contraction type. In one embodiment, the method 3100 is performed by the control unit 104 shown in FIG. 1, or a sub-unit therefore such as the model fitting unit 104-1.

At the step of obtaining 3102, a first waveform is obtained. The first waveform comprises a functional response of a first artificial tissue. The first waveform has a length corresponding to an expected length of a contraction-relaxation cycle of the first artificial tissue.

At the step of predicting 3104, a tissue contraction type is predicted from the first waveform using a classifier trained on a synthetically generated training data set (as described above in relation to FIG. 30).

At the optional step of outputting 3106, the tissue contraction type is output. In one embodiment, outputting the tissue contraction type comprises storing, or saving, the tissue contraction type to a persistent storage such as a non-volatile memory, a non-transitory medium, or the like. Additionally, or alternatively, outputting the tissue contraction type comprises transmitting the tissue contraction type via a network (e.g., a local area network, a wide area network, and the like), or displaying the tissue contraction type for review by a user.

FIG. 32 shows a method 3200 for training a parameter estimation model using synthetic training data according to an aspect of the present disclosure.

The method 3200 comprises the steps of obtaining 3202 a plurality of waveforms, extracting 3204 a plurality of parameter sets, determining 3206 a parameter set distribution, generating 3208 a synthetic training data set, and training 3210 a prediction model on the synthetic training data set. In one embodiment, the method 3200 is performed by the control unit 104, or a sub-unit thereof, shown in FIG. 1.

In many situations, the efficacy of prediction models (e.g., machine learning models such as deep neural networks) are limited by the amount and quality of training data available. In the absence of high-volume, high-quality, training data, prediction models will often fail to produce adequate outputs. In the present disclosure, this problem would lead to inaccurate contraction-relaxation models being fit which would subsequently reduce the effectiveness and applicability of such models being used in practice for tasks such as drug discovery and development. The method 3200 seeks to address such problems by generating high-fidelity synthetic training data thereby allowing an almost limitless amount of data to be generated. This helps to improve the performance of the prediction model being trained which subsequently improves the accuracy of the models fit using the prediction model. This improvement of accuracy helps drive improvements to downstream tasks which utilize features extracted from such models.

At the step of obtaining 3202, a plurality of waveforms are obtained. The plurality of waveforms comprise functional responses of one or more artificial tissues. The plurality of waveforms all comprise a common contraction type of a predetermined plurality of contraction types. For example, all of the waveforms are single contraction type waveforms or double contraction type waveforms. Therefore, the model trained by the method 3200 is trained to predict the parameters of either a single contraction type model or a double contraction type model depending on the common contraction type of the plurality of waveforms.

The plurality of waveforms correspond to the real, or bootstrap, data from which the synthetic training data set will be generated. Each waveform corresponds to a time-series of values which represent the functional response (e.g., contractile force, calcium transients, etc.) of an artificial, or engineered, tissue over one single contraction-relaxation cycle or one double contraction-relaxation cycle. Therefore, each waveform contains at least one contraction period and at least one relaxation period and may be parameterized according to either the single or double contraction-relaxation model described above in relation to FIGS. 3 and 25.

To help ensure that the synthetic training data is representative of as wide a population of contraction-relaxation cycles as possible, the plurality of waveforms are preferably obtained from a variety of artificial tissues across a range of different conditions. The artificial tissues comprise one or more engineered muscle tissues such as engineered cardiac tissue and/or engineered skeletal muscle tissue. Variety is achieved by obtaining waveforms from artificial tissues across a range of different cell lines, disease states, and treatments. Alternatively, the scope of conditions within the plurality of waveforms is restricted thereby allowing the synthetic data, and the subsequent parameter estimation model, to be finetuned to specific applications. For example, the plurality of waveforms may be restricted to vehicular treated waveforms to generate a control parameter estimation model or may be restricted to specific tissue types (e.g., engineered cardiac tissue) to generate a tissue-specific parameter estimation model. Beneficially, this helps improve the performance of the parameter estimation model when it is known which class of waveform the parameter estimation model will be used for.

At the step of extracting 3204, a plurality of parameter sets are extracted from the plurality of waveforms. A parameter set of the plurality of parameter sets characterizes a corresponding waveform of the plurality of waveforms.

A parameter set comprises the parameters of either the single or double contraction-relaxation cycle model (e.g., as described above in relation to FIGS. 3 and 25). As stated above, which model is used is dependent upon the common contract type of the plurality of waveforms obtained at the obtaining 3202 step described above. In one embodiment, a parameter set associated with a waveform comprises at least one maximum value parameter value (A), a shift parameter value (B), at least one contraction midpoint parameter value (t₀), at least one contraction growth rate parameter value (k_{c}), at least one relaxation midpoint parameter value (t_{d}), and at least one relaxation growth rate parameter value (kᵣ).

The plurality of parameter sets are extracted using either a supervised, unsupervised, or semi-supervised approach. According to the supervised approach, a parameter set is fit manually to each waveform. For example, a first waveform is presented to a user and the values of the parameters within the parameter set are adjusted by the user until a second waveform produced by a contraction-relaxation cycle model fit according to the parameter set closely matches the waveform. The final parameter set which results in the closely matching second waveform is then used as a parameter set within the plurality of parameter sets associated with the first waveform. According to the unsupervised approach, a parameter set is automatically fit to a waveform (e.g., using the approach described in relation to FIGS. 9 and 10 above). In one embodiment, the unsupervised approach utilizes a trained machine learning model to predict parameter set values for a waveform. According to the semi-supervised approach, the automatically determined parameter sets obtained according to the unsupervised approach are manually reviewed and refined by one or more users.

At the step of determining 3206, a parameter set distribution is determined from the plurality of parameter sets.

The plurality of parameter sets extracted at the extracting 3204 step of the method 3200 comprise a plurality of values for each parameter of the (single or double) contraction-relaxation cycle model. For example, if 100 parameters sets are extracted, then there will be 100 parameter values extracted for each parameter of the—contraction-relaxation cycle model. At the step of determining 3206, a distribution, or distribution of values, is determined for each parameter of the model. In one embodiment, a distribution is independently determined for each parameter. Alternatively, a multivariate distribution is determined for the plurality of parameters forming the parameter set.

Any suitable method may be used to determine the parameter set distribution such as histogram based approaches, density estimation approaches, and clustering approaches. In one embodiment, the parameter set distribution is determined using a kernel density estimation (KDE) method which utilizes a kernel and a bandwidth parameter to estimate the parameter set distribution. In one implementation, a normal (Gaussian) kernel is used with the bandwidth selected using either cross-validation or a bandwidth selection approach such as Scott's rule or Silverman's rule.

Once the parameter set distribution has been determined, a parameter set can be obtained by sampling from this distribution (i.e., sampling from each individual distribution or sampling from the joint distribution).

At the step of generating 3208, a synthetic training data set is generated. Each element of the synthetic training data set comprises a synthetic waveform and a corresponding parameter set used to generate the synthetic waveform. The corresponding parameter set is obtained from the parameter set distribution.

The synthetic training data set is generated by repeatedly sampling a parameter set for the contraction-relaxation cycle model from the parameter set distribution (as described above) and generating a corresponding waveform for each of the sampled parameter sets. In this way, a training data set of any size (e.g., 1000, 10000, 100000, etc. training data elements) can be generated efficiently. The synthetic data will also closely approximate real waveform data because the parameter set distribution is modelled on real world data.

The synthetic waveform will either be a single contraction type waveform (as illustrated and described in relation to FIG. 3) or a double contraction type waveform (as illustrated and described in relation to FIG. 25).

Optionally, a noise component is added to each of the waveforms in the synthetic training data set. The noise component is determined via a uniform distribution which is determined from the plurality of waveforms.

At the step of training 3210, a prediction model is trained using the synthetic training data set. The prediction model is trained to estimate an output parameter set from an input waveform. In one embodiment, the prediction model corresponds to the parameter estimation model described in relation to FIG. 6 above (i.e., the prediction model 600). As stated above, training the parameter estimation model in one embodiment comprises using minibatch gradient descent with a batch size of 128 and an ADAM solver. The ADAM solver has an initial learning rate of 1e-3 with early stopping based on validation loss. More details regarding the training of the parameter estimation model performed at the step of training 3210 is given above in relation to the description of FIG. 6.

FIG. 33 shows a method 3300 for extracting a single or double contraction-relaxation cycle waveform according to an aspect of the present disclosure.

The method 3300 comprises the steps of obtaining 3302 a first waveform, convolving 3304 the first waveform with a pulse train, identifying 3306 a first location, and extracting 3308 a second waveform from the first waveform at the first location. The method 3300 also comprises the optional step of outputting 3310 the second waveform. In one embodiment, the method 3300 is performed by the signal processing unit 104-2 of the control unit 104 shown in FIG. 1.

In general, the method 3300 extracts a single or double contraction-relaxation cycle waveform from a larger waveform comprising multiple contraction-relaxation cycles. The larger waveform may be obtained from a hardware device such as a bioreactor (i.e., the bioreactor 102 shown in FIG. 1) and will typically correspond to the functional response of an artificial tissue under certain conditions. For example, the larger waveform may comprise the contractile force response of an engineered cardiac tissue stimulated at 1 Hz over a period of 30 seconds. In this example, the larger waveform will comprise approximately 30 peaks, or 30 contraction-relaxation cycles, corresponding to the contractions of the engineered cardiac tissue in response to the electrical stimulation. The method 3300 provides an efficient and accurate mechanism for extracting each (single or double) contraction-relaxation cycle from the larger waveform such that these sub-waveforms may then be used for further processing and analysis (e.g., fitting a model to these waveforms and extracting relevant features as described in relation to FIG. 28 above).

At the step of obtaining 3302, a first waveform is obtained. The first waveform comprises a plurality of functional responses of an artificial tissue stimulated at a first frequency.

In one embodiment, the first waveform is obtained from a bioreactor (e.g., the bioreactor 102 shown in FIG. 1) in which the artificial, or engineered, tissue is grown/maintained. As stated previously, the artificial tissue comprises engineered muscle tissue such as engineered cardiac tissue or engineered skeletal muscle tissue.

As described above in relation to FIG. 1, an electrical stimulation is applied to a cell culture during maturation and, once matured, an electrical stimulation is applied to the artificial tissue to simulate a physiological environment that is native to the artificial tissue thereby allowing the functional response of the artificial tissue to this stimulation to be measured. The first waveform obtained at the step of obtaining 3302 comprises the functional responses of the artificial tissue in response to stimulation at the first frequency.

In one embodiment, the first frequency, or pacing frequency, at which the artificial tissue is stimulated is from 0.1 Hz to 20 Hz. In a further embodiment, the first frequency is from 1 Hz to 6 Hz.

Optionally, the method 3300 comprises the step of stimulating (not shown) the artificial tissue at the first frequency prior to the step of obtaining 3302 the first waveform. For example, an instruction (e.g., the instruction 126 in FIG. 1), or command, is sent to the bioreactor containing the artificial tissue (e.g., the bioreactor 102 in FIG. 1) to cause the bioreactor to stimulate the artificial tissue at the first frequency.

At the step of convolving 3304, the first waveform is convolved with a pulse-train to generate a convolved waveform. The pulse-train is generated at the first frequency.

The pulse-train corresponds to an idealized representation of the functional responses of the artificial tissue at the first frequency. As is known, a pulse-train, or pulse wave, is a waveform comprising non-sinusoidal (rectangular) pulses, or waves, of duration T with a frequency of 1/T₀, where T₀ is the period of the pulse-train. The duty cycle of the pulse-train is thus T/T₀. The pulse-train with which the first waveform is convolved therefore comprises a sequence of rectangular pulses with period 1/f and duration T where f is the first frequency.

Optionally, the method 3300 further comprises the step of generating (not shown) the pulse-train at the first frequency prior to performing the step of convolving 3304.

Given the first waveform, X₁, and the pulse-train, p, the convolution, y=(X₁*p), performed at the step of convolving 3304 is:

$$y [ k ] = ( {X}_{1} * p ) [ k ] = {\sum }_{n} {X}_{1} [ n - k ] p [ k ] . \qquad ( 7 )$$

At the step of identifying 3306, a first location associated with a maximum value of the convolved waveform is identified. The first location corresponds to an expected location of a first (single or double) contraction-relaxation cycle.

The maximum value of the convolved waveform corresponds to the location where the first waveform and the pulse-train best align. As such, the location of the maximum value of the convolved waveform is used to identify the most likely location of a single contraction-relaxation cycle within the first waveform. The location of the maximum value of the convolved waveform also provides an anchor point from which the other contraction-relaxation cycles can be extracted from the first waveform.

At the step of extracting 3308, a second waveform is extracted from the first location of the first waveform. The second waveform comprises the first contraction-relaxation cycle and has a first duration proportional to the first frequency. The second waveform is either a single contraction type (as illustrated in FIG. 3) or a double contraction type (as illustrated in FIG. 25).

The first location identified at the identifying 3306 step corresponds to the most likely location of one (single or double) contraction-relaxation cycle within the first waveform. The second waveform extracted from the first location thus comprises this contraction-relaxation cycle. Because the first waveform corresponds to the functional response of the artificial tissue when stimulated at a predetermined, set, frequency, the duration, or length, of the second waveform is proportional to this frequency. For example, if the artificial tissue was stimulated at 1 Hz then the first duration would be 1 s, if the artificial tissue was stimulated at 2 Hz then the first duration would be 0.5 s, etc.

Thus, the second waveform corresponds to a window, or subframe, within the first waveform having a length corresponding to the first duration. In one embodiment, the second waveform is centered at the first location such that a midpoint of the second waveform is aligned, or substantially aligned, to the first location of the first waveform.

At the optional step of outputting 3310, the second waveform is output. In one embodiment, outputting the second waveform comprises storing, or saving, the second waveform to a persistent storage such as a non-volatile memory, a non-transitory medium, or the like. Additionally, or alternatively, outputting the second waveform comprises transmitting the second waveform via a network (e.g., a local area network, a wide area network, and the like), or displaying the waveform for review by a user.

In one embodiment, outputting the second waveform comprises causing the second waveform to be output to another process or method of the present disclosure. For example, the second waveform may be output to the method 2800 described above such that the step of obtaining 2802 comprises obtaining the second waveform from the method 3300.

FIG. 34 shows a method 3400 for extracting a further single or double contraction-relaxation cycle from a waveform according to an embodiment of the present disclosure.

The method 3400 comprises the steps of identifying 3402 a second location, extracting 3404 a third waveform from the first waveform at the second location, and further comprises the optional step of outputting 3406 the third waveform. The method 3400 is performed after the method 3300. Particularly, the method 3400 may be performed after the step of identifying 3306 the first location and may be performed in parallel to the step of extracting 3308 the second waveform. In one embodiment, the method 3400 is performed by the signal processing unit 104-2 of the control unit 104 shown in FIG. 1.

The method 3400 is used to extract a further waveform comprising a single or double contraction-relaxation cycle from the first waveform. Beneficially, the extraction performed at the method 3400 is efficient and highly parallel because the method 3400 leverages prior information regarding the expected locations of the single or double contraction-relaxation cycles within the first waveform thus enabling the contraction-relaxation cycles to be extracted independently.

At the step of identifying 3402, a second location is identified based on the first location and the first frequency. The second location corresponds to an expected location of a second contraction-relaxation cycle.

The first location (identified at the identifying 3306 step of the method 3300) corresponds to the best alignment between the first waveform and the pulse-train. As such, the first location may be understood as the most likely location of a contraction-relaxation cycle within the first waveform. Because the first waveform comprises functional responses of the artificial tissue at a predetermined frequency (i.e., the first frequency), the other contraction-relaxation cycles, which are linked to the functional responses of the artificial tissue, are highly likely to be located at locations spaced from the first location. The first location can thus act as an anchor point within the first waveform from which the other contraction-relaxation cycle waveforms can be extracted.

The second location corresponds to an expected location of a second contraction-relaxation cycle (either single or double) and will be spaced from the first location by a distance proportional to the first frequency. Specifically, given the first location, t₁, within the first waveform, the second location, t₂, will be t₂=t₁+(a×T₀) where a is a step factor and T₀=1/f is the period of the pulse-train. Therefore, the next single or double contraction-relaxation cycle can be identified by setting the scaling factor to a=1 and the previous contraction-relaxation cycle can be identified by setting the scaling factor to a=−1.

At the step of extracting 3404, a third waveform is extracted from the second location of the first waveform. The third waveform comprises the second (single or double) contraction-relaxation cycle and has a second duration proportional to the first frequency.

The third waveform corresponds to a functional response (i.e., single or double contraction-relaxation cycle) of the artificial tissue when stimulated at a predetermined, set, frequency. Therefore, the duration, or length, of the third waveform is proportional to this frequency. For example, if the artificial tissue was stimulated at 1 Hz then the second duration would be 1 s, if the artificial tissue was stimulated at 2 Hz then the second duration would be 0.5 s, etc. In one embodiment, the first duration and the second duration are the same.

In one embodiment, the third waveform is centered at the second location such that a midpoint of the third waveform is aligned, or substantially aligned, to the second location of the first waveform.

At the optional step of outputting 3406, the third waveform is output. In one embodiment, outputting the third waveform comprises storing, or saving, the third waveform to a persistent storage such as a non-volatile memory, a non-transitory medium, or the like. Additionally, or alternatively, outputting the third waveform comprises transmitting the third waveform via a network (e.g., a local area network, a wide area network, and the like), or displaying the waveform for review by a user.

In one embodiment, outputting the third waveform comprises causing the third waveform to be output to another process or method of the present disclosure. For example, the third waveform may be output to the method 2900 described above such that the step of obtaining 2802 comprises obtaining the third waveform from the method 3400.

As stated above, the method 3200 of extracting a further single or double contraction-relaxation cycle waveforms from the first waveform may be repeated for all contraction-relaxation cycles within the first waveform. Because the extraction performed by the method 3400 depends only on the first location and the first frequency, no further signal processing or analysis is required to identify the locations of the further contraction-relaxation cycles. The method 3400 therefore provides a fast and efficient method for extracting single or double contraction-relaxation cycles from a waveform. These waveforms can then be processed further, e.g., by fitting a model to the waveform to generate a noise filtered representation of the waveform.

FIG. 35 shows a method 3500 for predicting a perturbation effect according to an aspect of the present disclosure.

The method 3500 comprises the steps of obtaining 3502 a plurality of signals, splitting 3504 the plurality of signals into a first plurality of waveforms, determining 3506 a predicted contraction type, fitting 3508 a model to each of the first plurality of waveforms, generating 3510 a second plurality of waveforms from the model, extracting 3512 a first feature value, extracting 3512 a second feature value, and determining 3516 an effect. In one embodiment, the method 3500 is performed by the control unit 104, or a sub-unit thereof, shown in FIG. 1.

In general, the method 3500 describes an application of the single and double contraction-relaxation cycle models to a downstream drug discovery/development task. Specifically, the contraction-relaxation cycle models are used to generate accurate feature values efficiently from a baseline signal and a perturbation signal of an engineered tissue. Accurately extracting features from these signals allows an effect associated with the perturbation to be efficiently and accurately identified.

At the step of obtaining 3502, a plurality of signals are obtained. The plurality of signals comprise a baseline signal and a perturbation signal. The baseline signal comprises a first plurality of functional responses of an engineered tissue under reference or baseline conditions. The perturbation signal comprises a second plurality of functional responses of the engineered tissue under a first set of perturbation conditions.

The baseline signal and the perturbation signal comprise a plurality of functional responses (i.e., a plurality of contraction-relaxation cycles, or peaks) of the engineered tissue under reference and a first set of perturbation conditions. In general, reference conditions refer to conditions which provide a baseline comparison to the perturbation conditions. In embodiments, a reference condition is a condition associated with a control setup or environment. A reference condition may correspond to an engineered, or artificial, tissue in its default, natural, or unaltered state (i.e., without dosage of a drug or agent).

Alternatively, a reference condition may correspond to vehicle treated engineered tissue.

Perturbation conditions refer to conditions in which the engineered tissue has been perturbed in some way. Examples of perturbation conditions include the administration of a drug or compound (i.e., a perturbant), a disease state, a different cell line, a physical perturbance applied to the engineered tissue, or a change to the environment of the engineered tissue. As such, perturbation conditions may alternatively be referred to as treatment conditions. In the case of perturbations involving a drug or compound, the conditions are further associated with an effect related to the drug or compound such as a mechanism of action or a toxicity. Given the range of different perturbation conditions, an engineered tissue may be associated with more than one perturbation condition (e.g., a diseased engineered tissue having been treated with a specific compound).

In one embodiment, the baseline signal and the perturbation signal are obtained from a bioreactor (e.g., the bioreactor 102 of FIG. 1) in which the engineered, or artificial, tissue is grown/maintained. As stated previously, the artificial tissue comprises engineered muscle tissue such as engineered cardiac tissue or engineered skeletal muscle tissue. The baseline signal and the perturbation signal are obtained at two different time points. For example, the baseline signal is obtained at a first time point, the engineered tissue is then perturbed according to the first perturbation (e.g., a first dosage of a compound is applied to the engineered tissue), and the perturbation signal is obtained at a second time point subsequent the first time point. The baseline signal and the perturbation signal comprise the functional response of the engineered tissue when stimulated at a predetermined pacing frequency (e.g., 0.1 Hz, 0.5 Hz, 1 Hz, 2 Hz, etc.). Alternatively, the baseline signal and the perturbation signal comprise the spontaneous functional response of the engineered tissue in the absence of external stimulation.

At the step of splitting 3504, the plurality of signals are split into a first plurality of waveforms. Each waveform of the first plurality of waveforms comprises at least one contraction response and at least one relaxation response of the engineered tissue during a single contraction-relaxation cycle.

The plurality of signals are split into a first plurality of waveforms using the method 3300 for extracting a contraction-relaxation cycle waveform described above. Alternatively, the plurality of signals are split by manually annotating or extracting the regions within the first plurality of waveforms which correspond to individual contraction-relaxation cycles.

The first plurality of waveforms generated at the step of splitting 3504 comprise a first subset of waveforms associated with waveforms extracted from the baseline signal and a second subset of waveforms associated with waveforms extracted from the perturbation signal. The subsequent model fitting (described below) is independent of the source of the waveform (i.e., independent of whether the waveform is a reference of perturbation waveform), but the identification of whether a waveform corresponds to reference or perturbation condition is used in the subsequent feature extraction steps.

At the step of determining 3506, a predicted contraction type of a plurality of contraction types is determined for each of the first plurality of waveforms. The plurality of contraction types include a single contraction type and a double contraction type.

The predicted contraction type is determined by a classifier based on a waveform input to the classifier. As described in more detail above in relation to FIGS. 6 and 7A-7D, the classifier comprises a trained machine learning model such as a trained neural network.

In one embodiment, the classifier comprises a convolutional neural network, a dilated convolutional neural network, and a long short-term memory network. Further architectural details of the classifier are given in relation to FIGS. 6 and 7A-7D above.

At the step of fitting 3508, a model is fit to each waveform of the first plurality of waveforms based on a corresponding predicted contraction type. For example, if a waveform is a single contraction type, then a single contraction type model is fit at the step of fitting 3508. Alternatively, if a waveform is a double contraction type, then a double contraction type model is fit at the step of fitting 3508. The model independently parameterizes growth of the at least one contraction response and the at least one relaxation response of the engineered tissue.

The step of fitting 3508 the model to each waveform corresponds to the step of fitting 2806 described in more detail above in relation to FIG. 28. Consequently, at the step of fitting 3508, the step of fitting 2806 is repeated for each waveform in the first plurality of waveforms. The process of fitting a model to a waveform is described in more detail above in relation to FIGS. 28 and 29.

At the step of generating 3510, a second plurality of waveforms are generated from the model fit to each waveform of the first plurality of waveforms. The second plurality of waveforms comprise a plurality of filtered baseline waveforms associated with the baseline signal and a plurality of filtered perturbation waveforms associated with the perturbation signal.

The step of generating 3510 corresponds to repeatedly applying the step of generating 908 (described in more detail above in relation to FIG. 28) to the model fit to each waveform in the first plurality of waveforms. The process of generating a second waveform from a model fit to a first waveform is described in more detail above in relation to FIGS. 28 and 29 above.

At the step of extracting 3512, a first feature value of a first feature is extracted from the plurality of filtered baseline waveforms.

The plurality of filtered baseline waveforms comprise noise filtered, or noise suppressed, representations of the contraction-relaxation cycles in the baseline signal. Because the signals have been filtered to remove noise, features can be accurately extracted from these waveforms.

The step of extracting 3512 the first feature value comprises extraction a plurality of feature values from the plurality of filtered baseline waveforms such that the first feature value comprises the plurality of feature values, or a representation of the plurality of feature values. As such, a value of the first feature is extracted from each of the plurality of filtered baseline waveforms to determine to first feature value. In one embodiment, the first feature value comprises an average (mean, median, etc.) value of the first feature determined from the plurality of filtered baseline waveforms. In a further embodiment, the first feature value comprises a maximum value, minimum value, or distribution of values determined from the plurality of filtered baseline waveforms.

The first feature is one of a twitch, or peak, amplitude (i.e., the peak amplitude 206 shown in FIG. 2), a contraction time (i.e., the time to peak amplitude 208 shown in FIG. 2), a maximum contraction slope (i.e., the maximum rate of development 214 shown in FIG. 2), a relaxation time (i.e., the time to peak decline 210 shown in FIG. 2), a maximum relaxation slope (i.e., the maximum rate of declination 216 shown in FIG. 2), or a twitch duration (i.e., the duration 212 shown in FIG. 2).

In one embodiment, the first feature is a total number of single contraction type waveforms or a total number of double contraction type waveforms (as described above in relation to FIG. 27). As such, the first feature value corresponds to the number or distribution of single and/or double contraction type waveforms within the plurality of filtered baseline waveforms.

At the step of extracting 3514, a second feature value of the first feature is extracted from the plurality of filtered perturbation waveforms.

The step of extracting 3514 the second feature value comprises extraction a plurality of feature values from the plurality of filtered perturbation waveforms such that the second feature value comprises the plurality of feature values, or a representation of the plurality of feature values. As such, a value of the first feature is extracted from each of the plurality of filtered perturbation waveforms to determine to second feature value. In one embodiment, the second feature value comprises an average (mean, median, etc.) value of the first feature determined from the plurality of filtered perturbation waveforms. In a further embodiment, the second feature value comprises a maximum value, minimum value, or distribution of values determined from the plurality of filtered perturbation waveforms.

As stated above, in one embodiment, the first feature is a total number of single contraction type waveforms or a total number of double contraction type waveforms such that the second feature value corresponds to the number or distribution of single and/or double contraction type waveforms within the plurality of filtered perturbation baseline waveforms.

At the step of determining 3516, an effect associated with the first perturbation is determined based on a comparison of the first feature value and the second feature value.

The first feature value is a quantitative descriptor of the functional response of the engineered tissue under reference conditions. The second feature value is a quantitative descriptor of the functional response of the engineered tissue under perturbation conditions involving the first perturbation. As such, a comparison of the first and second feature values reveals any change in functional response of the engineered tissue, or effect, occurring as a result of the first perturbation. One example of such a comparison is given above in relation to FIG. 27.

As a further example, the first perturbation may correspond to an application of a compound having an unknown physiological effect. A comparison of the first feature value-corresponding in this example to the peak amplitude of a contractile force waveform of the engineered tissue under reference conditions- and the second feature value-corresponding to the peak amplitude of a contractile force waveform of the engineered tissue under perturbation conditions involving an application of the compound-reveals an increase in average peak amplitude. Consequently, it can be inferred that the compound has an effect associated with increasing the contractile force of the engineered tissue during contraction-relaxation cycles. Beneficially, because the feature values are determined from noise filtered waveforms, effects arising due to the difference between the feature values can be more accurately identified leading to improved processing and potentially improved patient outcomes.

### Systems and Methods for Tissue Scaffold Tracking.

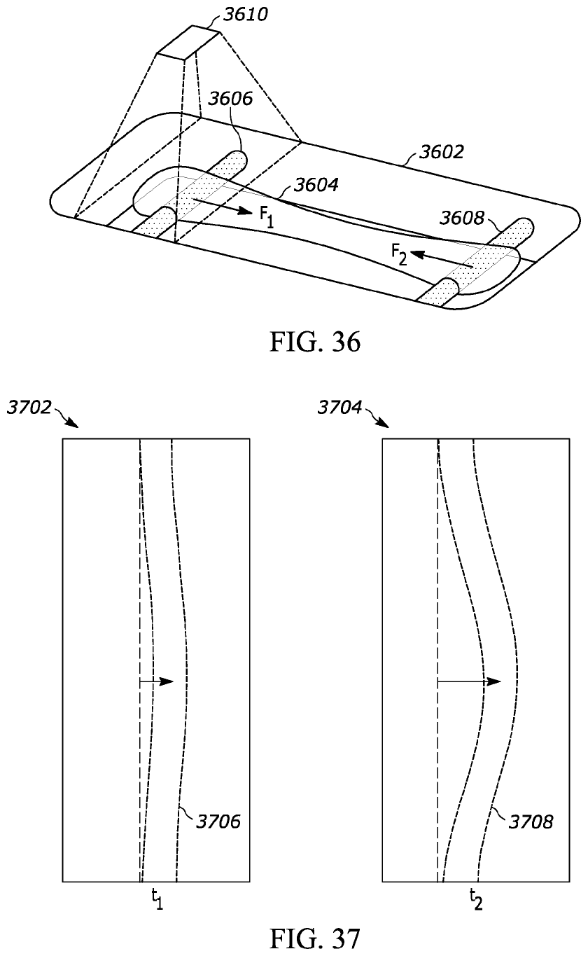

FIG. 36 shows a well 3602 of a bioreactor, such as the bioreactor 102 shown in FIG. 1, according to an embodiment of the present disclosure.

The well 3602 contains an artificial tissue 3604 (or engineered tissue) which is attached to a first flexible scaffold 3606 and a second flexible scaffold 3608. FIG. 36 further shows an imaging device 3610 configured to obtain one or more images of the artificial tissue 3604, the first flexible scaffold 3606, and/or the second flexible scaffold 3608. In one embodiment, the well 3602, the artificial tissue 3604, the first flexible scaffold 3606, the second flexible scaffold 3608, and the imaging device 3610 correspond respectively to the well 114, the engineered tissue 124, the first scaffold 120-1, the second scaffold 120-2, and an imaging device or optical sensor of the sensor assembly 108 shown in FIG. 1.

As stated above in relation to FIG. 1, the artificial tissue 3604 (or engineered tissue) is grown within the well 3602 from cells seeded therein. During maturation, the artificial tissue 3604 attaches to the first flexible scaffold 3606 and/or the second flexible scaffold 3608. Both the first flexible scaffold 3606 and the second flexible scaffold 3608 are disposed across the well 3602 thereby permitting the artificial tissue 3604 to attach thereto. The first flexible scaffold 3606 and the second flexible scaffold 3608 comprise a flexible element which is arranged to deflect, or deform, in response to a force exerted thereon. For example, the first flexible scaffold 3606 is arranged to deform in a first direction in response to a contractile force, F₁, exerted on the first flexible scaffold 3606 by the artificial tissue 3604. Similarly, the second flexible scaffold 3608 is arranged to deform in a second direction in response to a contractile force, F₂, exerted on the second flexible scaffold 3608 by the artificial tissue 3604. In a further example, the first flexible scaffold 3606 and the second flexible scaffold 3608 are arranged to deflect in response to a predetermined force exerted thereon by a probe or other instrument. In one embodiment, the first flexible scaffold 3606 and the second flexible scaffold 3608 are formed of a flexible polymer wire such as a poly(octamethylenemaleate(anhydride)citrate) (POMaC) wire. In such an embodiment, the flexible scaffolds may be referred to as wires or flexible wires.

The imaging device 3610 is configured to detect a deformation, or deflection, of the first flexible scaffold 3606 and/or the second flexible scaffold 3608 (e.g., occurring as a result of contractile force exerted on the flexible scaffolds by the artificial tissue 3604 or a probe).

The imaging device 3610 is configured to obtain a plurality of image-based representations of the one or more deflections of the first flexible scaffold 3606 and/or the second flexible scaffold 3608 over a time frame or time period. For example, the imaging device 3610 may be configured to capture an image, or frame, of a tissue or a region of the tissue which is attached to a tissue scaffold, every n seconds. Here, n is associated with a predetermined rate at which the images or frames are to be captured. For example, when n=1 then one image is captured per second. Any suitable value of n may be chosen such as n={1/60, 1/50, 1/30, 1/24, 1/12, 1/4, 1/2, 1,2} and the like. In one embodiment, the frame rate is determined according to the frequency at which the tissues within the device are being stimulated. The sequences of images or frames of a tissue over a time frame therefore captures one or more deflections of the first flexible scaffold 3606 and/or the second flexible scaffold 3608 over the time frame. The image(s) captured by the imaging device 3610 may be output from the device or bioreactor to a control unit or processing unit (e.g., the image(s) captured by the sensor assembly 108 may be output to the control unit 104 shown in FIG. 1).

FIG. 37 shows example images of tissue scaffolds under different contractile forces according to embodiments of the present disclosure.

FIG. 37 shows a first image 3702 and a second image 3704 obtained from an imaging device of a bioreactor (e.g., the imaging device 210 shown in FIG. 36). The first image 3702 captures a first deflection of a tissue scaffold 3706 at a first time point t₁. The second image 3704 captures a second deflection of the tissue scaffold 3708 at a second time point t₂. To appreciate the difference in deflections, the deflections are shown in relation to a common reference line (the vertical dashed lines in both the images). The change in deflection between t₁ and t₂ may be used to encode or otherwise characterize a contractile force exerted on the tissue scaffold (e.g., by a tissue attached to the tissue scaffold or by a probe or other instrument).

The present disclosure is directed to flexible scaffold tracking which allows for accurately and efficiently extracting and measuring deflections of tissue scaffolds thereby allowing models which characterize the contractile force(s) which produced the deflections to be generated. Beneficially, such models may encode the contractile response of an engineered tissue thereby providing a quantification of a functional response of the engineered tissue. Alternatively, such models may be used to encode or calibrate the relationship between contractile force and measured displacement thereby improving the accuracy of contractile force measurements obtained from such models. Improvements to such models provide improvements to the downstream tasks which utilize such models whilst also improving the efficiency and performance of computing systems which are used to generate and deploy such models.

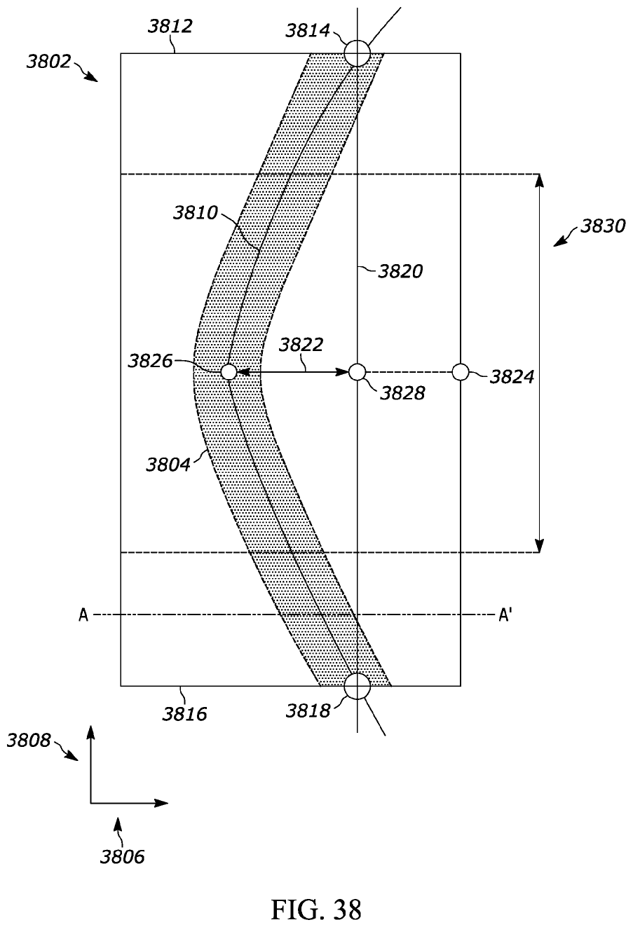

In general, flexible scaffold tracking may begin by obtaining an image from a device or bioreactor (e.g., from the bioreactor 102 of FIG. 1) of a flexible scaffold taken at a first time point. The image captures a deflection of the flexible scaffold along a first dimension due to a (contractile) force exerted thereupon at the first time point. A curve is fit to the image such that the curve extends along a centerline, or along an approximate centerline, of the flexible scaffold within the image. A displacement value is determined between the curve and a reference line extending along a second dimension perpendicular to the first dimension. A model is generated based on the displacement value such that the model characterizes the (contractile) force exerted on the flexible scaffold at the first time point. This approach is illustrated in FIG. 38 and further described in detail below.

FIG. 38 shows an approach for flexible scaffold tracking according to an aspect of the present disclosure.

FIG. 38 shows an image region 3802 of an image comprising a flexible scaffold 3804 taken at a first time point. The image region 3802 captures a deflection of the flexible scaffold 3804 along a first dimension 3806 due to a contractile force exerted on the flexible scaffold 3804 at the first time point. Also shown in FIG. 38 is a second dimension 3808 which is perpendicular to the first dimension 3806. In one embodiment, the first dimension 3806 corresponds to a vertical axis of the image region 3802 and the second dimension 3808 corresponds to a horizontal axis of the image region 3802. A curve 3810 is shown extending along a centerline of the flexible scaffold 3804. The curve 3810 intersects a first edge 3812 of the image region 3802 at a first intersection point 3814 and intersects a second edge 3816 of the image region 3802 at a second intersection point 3818. A reference line 3820 extends along the second dimension 3808 between the first intersection point 3814 and the second intersection point 3818. A measurement 3822 taken between the curve 3810 and the reference line 3820 corresponds to a displacement value for the flexible scaffold 3804 at the first time point. The measurement 3822 is determined at a reference point 3824 along the second dimension 3808. The reference point 3824 is determined such that the orthogonal distance along the first dimension 3806 between the curve 3810 and the reference line 3820 is maximal at the reference point 3824 (i.e., the distance between a first point 3826 on the curve 3810 at the reference point 3824 along the second dimension 3808 and a second point 3828 on the reference line 3820 at the reference point 3824 along the second dimension 3808 corresponds to the maximum orthogonal distance between the curve 3810 and the reference line 3820 along the first dimension 3806). FIG. 38 further shows a predetermined expected range 3830 within which the maximum orthogonal distance between the curve 3810 and the reference line 3820 is expected to lie. The predetermined expected range 3830 comprises a portion of a length of the image region 3802 along the second dimension 3808 (e.g., 60% of the height of the image region 3802). As will be described in more detail below, the displacement value determined from the measurement 3822 is used to generate a model which characterizes the contractile force exerted on the flexible scaffold 3804 (e.g., due to a contraction of an engineered tissue attached to the flexible scaffold 3804 or a predetermined force exerted on the flexible scaffold 3804 by a probe or other instrument).

The image region 3802 comprises an image, or a portion of an image, obtained from a device or bioreactor (e.g., obtained from an imaging device of a bioreactor such as the imaging device 210 shown in FIG. 36). The image region 3802 comprises a grayscale bright-field or fluorescence image. As shown more clearly in FIG. 36, the image region 3802 captures one of the tissue scaffolds of a device or bioreactor. The image region 3802 may be captured such that only the portion of the device containing the relevant tissue scaffold is captured within the image region 3802. Alternatively, the image region 3802 may be cropped from a larger image of the entire device/well or tissue. In such setting, the image region 3802 may be manually or automatically cropped (e.g., by cropping the larger image to a predetermined bounding box having co-ordinates known to correspond to the portion of larger image containing a tissue scaffold).

As stated above, the curve 3810 extends along the centerline, or the approximate centerline, of the flexible scaffold 3804 as captured within the image region 3802. To determine the centerline of the flexible scaffold 3804 within the image region 3802 one or more image processing operations are used to enhance the structure and/or appearance of the flexible scaffold 3804 within the image region 3802. As can be seen from the example images in FIG. 37, a flexible scaffold (e.g., the tissue scaffold 3706 or the tissue scaffold 3708) appears as a vessel like structure within the image. As such, image processing and/or filtering operations may be used to enhance the regions of the image that contain the vessel like structure.

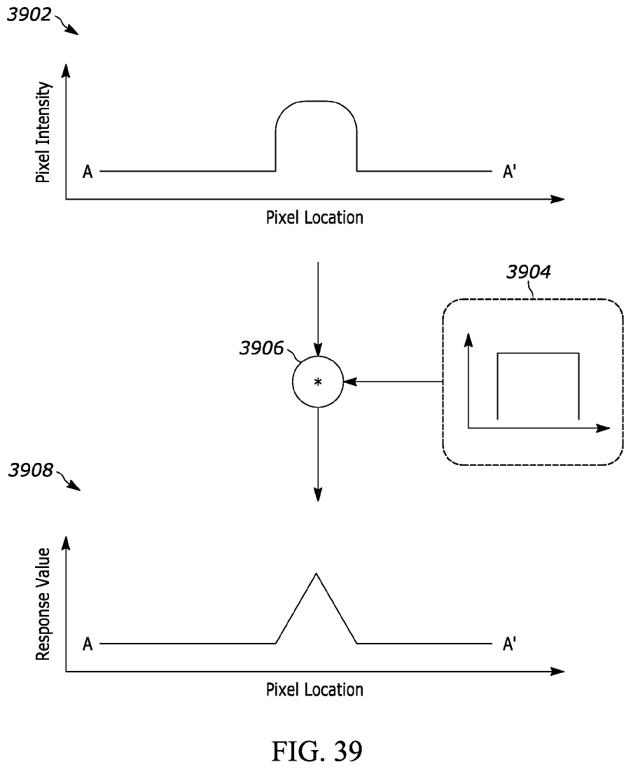

In one embodiment, the image region 3802 is transformed using a filtering operation to generate a transformed image region. The filtering operation determines a likelihood that a region or portion (i.e., a pixel or neighborhood of pixels) of the image region 3802 contains the flexible scaffold 3804. The transformed image region thus comprises a transformed representation of the image region 3802 where each pixel value in the transformed image region corresponds to the likelihood that the pixel value lies on a flexible scaffold. The filtering operation comprises a vessel enhancement filter such as a multiscale vessel enhancement filter (e.g., a Hessian-based Frangi vesselness filter) or diffusion filtering (e.g., a coherence-enhancing diffusion filter). In an embodiment, the filtering operation comprise convolving a one-dimensional kernel across one dimension of the image region 3802 as illustrated in FIG. 39.

FIG. 39 shows a one-dimensional vessel enhancement filter according to an embodiment of the present disclosure.

FIG. 39 shows a plot 3902 of pixel intensity values taken along the cross-section of the image region 3802 shown in FIG. 38 between the points A and A′. FIG. 39 further shows a one-dimensional kernel 3904, a convolution operation 3906, and a plot 3908 of response values. The one-dimensional kernel 3904 has a shape corresponding to an approximate cross sectional shape of the flexible scaffold. The shape of the one-dimensional kernel 3904 is square shaped, or approximately square shaped. Convolving, via the convolution operation 3906, the one-dimensional kernel 3904 across the pixel intensity values shown in the plot 3902 results in the response values shown in the plot 3908. The response values shown in the plot 3908 correspond to the pixel intensity values for the row of pixels from A to A′ in the transformed representation of the image region.

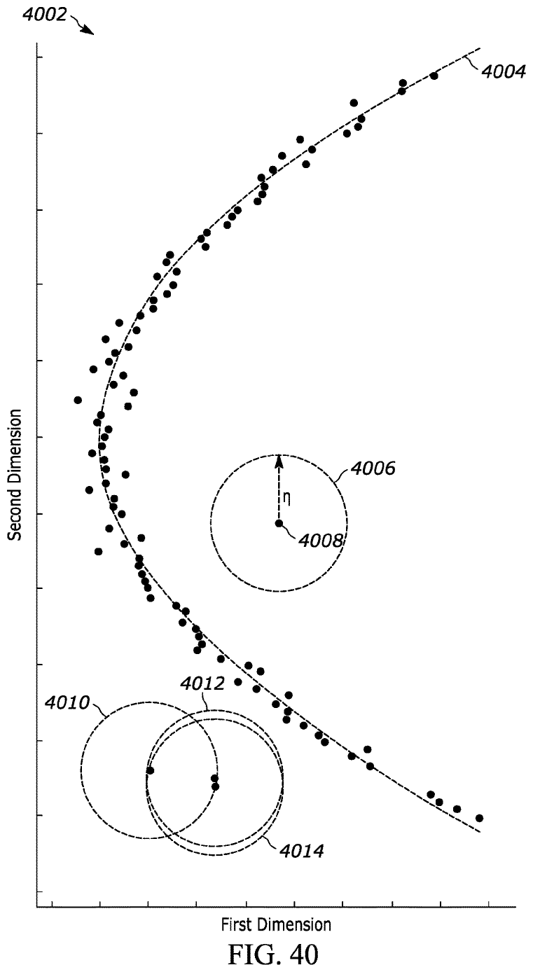

As can be seen from the plot 3908 of response values, the pixel location corresponding to the approximate center point of the flexible scaffold comprises the greatest response value as a result of the convolution operation 3906. Consequently, repeating the one-dimensional convolution across all rows of the image region 3802 shown in FIG. 38 results in a transformed image having maximum pixel intensities approximately along the centerline of the flexible tissue scaffold. Therefore, the pixel within each row of the transformed image having maximum intensity value (within the row) is chosen as the most likely estimate of the pixel along the centerline of the tissue scaffold. Repeating this for each row in the transformed image produces a plurality of data points corresponding to likely locations along the centerline of the flexible scaffold 3804 within the image region 3802. As shown in FIG. 40 and described below, a curve may be fit to the plurality of data points to determine the approximate centerline of the flexible scaffold 3804.

FIG. 40 shows a plot 4002 of data points obtained from a transformed image of a tissue scaffold according to an embodiment of the present disclosure.

The plot 4002 comprises a plurality of data points (represented as black dots within the plot 4002) corresponding to pixel locations having maximum local intensity (i.e., maximum intensity within the pixel's row). The plot 4002 further comprises a curve 4004 fit to the plurality of data points, a first neighborhood 4006 around a first outlier point 4008, and a plurality of further neighborhoods 4010-4014.

The curve 4004 corresponds to a quadratic curve fit to the plurality of data points (i.e., the locations of maximum pixel values within the transformed image along the first dimension, as described above in relation to FIG. 39). In an embodiment, the curve 4004 is fit to the plurality of data points using a least squares approach such that the sum of squares of the residuals—the deviations between the plurality of data points and a set of corresponding data points on the curve—is minimized. Once fit, the curve 4004 corresponds to the approximate centerline of the flexible scaffold.

In one embodiment, one or more outlier points are removed from the plurality of data points prior to the curve 4004 being fit. Beneficially, this helps to improve the accuracy of the curve fitting process thus resulting in a centerline that more closely corresponds to the centerline of the tissue scaffold. A data point of the plurality of data points is considered an outlier point if the outlier point meets or exceeds a predetermined outlier threshold.

In one embodiment, the predetermined outlier threshold comprises a neighborhood distance threshold. In the example shown in the plot 4002, the first outlier point 4008 is determined to be an outlier point because the distance between the first outlier point 4008 and each of the plurality of data points exceeds a predetermined distance threshold, n. That is, no other data points from the plurality of data points lie within the first neighborhood 4006 around the first outlier point 4008. The predetermined distance threshold η is relative to an expected width of the flexible scaffold. For example, if the flexible scaffold has an expected width of w pixels or μm (e.g., 50 μm, 75 μm, 100 μm, etc.), then the distance threshold is set as η=aw where α is a parameter which may take a value of between 1 and 2. In one implementation, α=1.5. Additionally, or alternatively, a data point is considered an outlier point if fewer than a predetermined number of other data points lie within the neighborhood of that data point. In the example shown in the plot 4002, the data points within each of the plurality of further neighborhoods 4010-4014 are visually identifiable as outlier points, but the neighborhoods are nevertheless overlapping. For example, the data points relating to the neighborhoods 4012 and 4014 lie within the neighborhood 4010. As such, a data point may be considered an outlier point if fewer than m other data points lie within the neighborhood around the data point (as defined by the distance threshold η). Here, m is a predetermined value such that 0<m<10, or 1<m<15. In one implementation, m=2.

Additionally, or alternatively, the predetermined outlier threshold comprises a pixel intensity threshold such that a pixel value at an image location corresponding to a location of an outlier point exceeds a predetermined intensity threshold. In one implementation, the predetermined intensity threshold is 3 times the median absolute deviation (MAD) of all pixel intensities for pixels lying along the tissue scaffold.

Referring once again to FIG. 38, the curve 3810 fit to the flexible scaffold 3804 as described above is used to determine the measurement 3822 corresponding to the displacement value for the flexible scaffold 3804 at the first time point.

The displacement value encodes the deflection of the flexible scaffold 3804 at the first time point relative to the expected position of the flexible scaffold 3804 when at rest. That is, the displacement or deflection of the flexible scaffold 3804 at a given time point corresponds to the difference between the position of the flexible scaffold 3804 at the given time point and the expected position of the flexible scaffold 3804 when at rest. The expected position of the flexible scaffold 3804 when at rest is modelled by the reference line 3820 such that the distance between the curve 3810 (extending along the centerline of the flexible scaffold 3804) and the reference line 3820 is used to determine the displacement value for the flexible scaffold 3804 at the first time point.

The reference line 3820 (alternatively referred to as the resting line, the baseline, or the expected resting position) is determined using the points of intersection of the curve 3810 and the boundaries, or edges, of the image region 3802 such that the reference line 3820 extends between the points of intersection. In FIG. 38, the first intersection point 3814 is determined by identifying the point of intersection between the curve 3810 and the first edge 3812 of the image region 3802. The second intersection point 3818 is determined by identifying the point of intersection between the curve 3810 and the second edge 3816 of the image region 3802. Here, the second edge 3816 of the image region 3802 is opposite the first edge 3812 of the image region 3802. The reference line 3820 is then fit such that the reference line 3820 extends between the first intersection point 3814 and the second intersection point 3818. In one embodiment, the reference line 3820 defines the second dimension 3808 (i.e., defines the angle/direction of the second dimension 3808 relative to the axis or axes of the image region 3802). In some cases, the reference line 3820 may align, or substantially align, with an image axis (e.g., the vertical axis) such that the second dimension 3808 is aligned, or substantially aligned, with the image axis. In some other cases, the reference line 3820 is misaligned with either of the image axes such that the second dimension 3808 does not substantially align with either of the image axes.

The measurement 3822, which is used to determine the displacement value of the flexible scaffold 3804 at the first time point, is then calculated between the curve 3810 and the reference line 3820. The measurement 3822 is determined at the reference point 3824 along the second dimension 3808. As stated above, the reference point 3824 is determined such that the orthogonal distance along the first dimension 3806 (which is perpendicular to the second dimension 3808) between the curve 3810 and the reference line 3820 is maximal at the reference point 3824 (i.e., the distance between a first point 3826 on the curve 3810 at the reference point 3824 along the second dimension 3808 and a second point 3828 on the reference line 3820 at the reference point 3824 along the second dimension 3808 corresponds to the maximum orthogonal distance between the curve 3810 and the reference line 3820 along the first dimension 3806). Alternatively, the displacement value is determined using a measurement corresponding to an estimated area of a shape bounded by the curve 3810 and the reference line 3820—i.e., the shape bounded by the path from the first intersection point 3814 along the curve 3810 to the second intersection point 3818 and closed by the path from the second intersection point 3818 to the first intersection point 3814 along the reference line 3820.

In one embodiment, a reference point determined using the process described above is replaced with a predetermined reference point if the reference point lies outside of the predetermined expected range 3830. As stated previously, the predetermined expected range 3830 corresponds to the portion of the image region 3802 within which the reference point 3824 is expected to lie—i.e., the portion of the image region 3802 which is expected to comprise the maximum orthogonal distance between the curve 3810 and the reference line 3820. The predetermined expected range 3830 thus comprises a proportion of a length of the image region 3802 along the second dimension 3808 which is centralized along the second dimension 3808. The proportion is from 20% to 80% of the length of the image region 3802 along the second dimension 3808. In one implementation, the proportion is 60% of the length of the image region 3802 along the second dimension 3808. The predetermined reference point used to replace the reference point if the reference point lies outside of the predetermined expected range 3830 may be the midpoint of the length of the image region 3802 along the second dimension 3808.

As stated above, the displacement value of the flexible scaffold 3804 at the first time point comprises the measurement 3822 between the curve 3810 and the reference line 3820. Using this displacement value, a model which characterizes the contractile force(s) exerted on the flexible scaffold 3804 may be generated. In one embodiment, the generated model comprises a scaffold deflection value for the first time point. The scaffold deflection value may be a distance value, or pixel-based distance value, determined from the displacement value. Alternatively, the scaffold deflection value may be a force value determined from the displacement value using a predetermined force-displacement model. In a further embodiment, the generated model comprises a force-displacement model operable to estimate a force value from an input scaffold displacement value.

The above description of FIGS. 38-40 is in relation to the generation of a model from a single image, or image region, of a tissue scaffold. According to an aspect of the present disclosure, the above described processes and approaches are used to generate a model based on a plurality of displacement values determined from a plurality of images of a flexible scaffold over a plurality of time points.

More particularly, a plurality of images of a flexible scaffold are obtained (e.g., from an imaging device or optical sensor of a bioreactor such as the bioreactor 102 shown in FIG. 1). Each image within the plurality of images captures a deflection, or displacement, of the flexible scaffold at a respective time point. As shown in FIG. 38 and described in more detail above, the deflection of the flexible scaffold occurs along a first dimension (e.g., the first dimension 3806 which may correspond to a horizontal axis of the plurality of images) due to a force, or contractile force, exerted on the flexible scaffold at the respective time point. Therefore, the plurality of images capture the change in tissue scaffold deflection over a plurality of time points due to (contractile) forces exerted on the tissue scaffold over the plurality of time points. The forces exerted on the tissue scaffold may occur due to contractile forces exerted on the flexible scaffold by a biological tissue attached to the tissue scaffold (e.g., an engineered or artificial tissue attached to the tissue scaffold during maturation within a bioreactor such as the bioreactor 102 shown in FIG. 1). Alternatively, the forces exerted on the tissue scaffold may occur due to predetermined forces exerted on the tissue scaffold by a probe or other instrument. For example, at time point t₁ a force of 50 μN is exerted on the flexible wire by a probe whilst at time point t₂ a force of 100 μN is exerted on the flexible wire by the probe.

A curve is fit to the tissue scaffold represented in each image of the plurality of images using the approach described in relation to FIG. 38. That is, for each image of the plurality of images, the approach described in relation to FIG. 38 is applied to generate a curve which extends along, or near, the centerline of the tissue scaffold as capture within the respective image. In one embodiment, the reference line (e.g., the reference line 3820 shown in FIG. 38) is determined using average intersection points determined across the plurality of images. Consequently, the reference line is constant across the plurality of images (i.e., the reference line does not move, or change, from image to image). A first average intersection point is determined between the plurality of curves and a first edge of an image region within the plurality of images (e.g., the first edge 3812 of the image region 3802 shown in FIG. 38) and a second average intersection point is determined between the plurality of curves and a second edge of an image region within the plurality of images (e.g., the second edge 3816 of the image region 3802 shown in FIG. 38). Here, an average intersection point comprises the median point of intersection between the plurality of curves and a respective edge of an image region. Alternatively, the average intersection point comprises the mean point of intersection. The reference line is then fit using the average intersection points such that the reference line extends between the first average intersection point and the second average intersection point. In one embodiment, the reference line defines the second dimension (i.e., defines the angle/direction of the second dimension relative to the axes of the plurality of images). In some cases, the reference line may align, or substantially align, with an image axis of the plurality of images (e.g., the vertical axis) such that the second dimension is aligned, or substantially aligned, with the image axis. In some other cases, the reference line is misaligned with the image axes such that the second dimension does not substantially align with any image axes of the plurality of images.

A plurality of displacement values are then determined from the plurality of curves and the reference line. Each of the plurality of displacement values comprise a measurement between a respective curve of the plurality of curves and the reference line (as described in detail above in relation to FIG. 38). Each of the plurality of displacement values are determined at a reference point along the second dimension of the plurality of images (e.g., the second dimension 3808 shown in FIG. 38 which may correspond to a vertical axis of the plurality of images). The reference point is determined from the plurality of curves such that an orthogonal distance along the first dimension (e.g., the first dimension 3806 shown in FIG. 38 which is perpendicular to the second dimension 3808) between an average curve and the reference line is maximal at the reference point. Here, the average curve corresponds to the median or mean curve determined from the plurality of curves. As stated above in relation to FIG. 38, if the reference point falls outside of a predetermined expected range—e.g., a proportion of a length of the plurality of images along the second dimension—then the reference point is replaced by a predetermined reference point such as the midpoint of the length of the plurality of images along the second dimension.

The plurality of displacement values are used to generate a model which characterizes the contractile forces exerted on the flexible scaffold over the plurality of time points. As will be described in more detail below, the generated model is either a time-series of scaffold deflection values or a force-displacement model. A model comprising a time-series of scaffold deflection values is generated from a plurality of displacement values determined from a plurality of images capturing deflections of a flexible scaffold due to contractile forces exerted thereupon by a biological tissue (e.g., a natural tissue, engineered tissue, or artificial tissue). The model thus characterizes the contractile forces of the biological tissue over the plurality of time points. Such a model may be utilized in various downstream tasks to encode the functional behavior of the biological tissue. That is, one or more features may be extracted from the model (i.e., from the time-series of contractile force values) and subsequently used to represent the functional response of the biological tissue over the plurality of time points. Thus, improving the accuracy and efficiency of the model generation process thus leads to improvements in the accuracy and efficiency of the representation of the functionality response determined from the model.

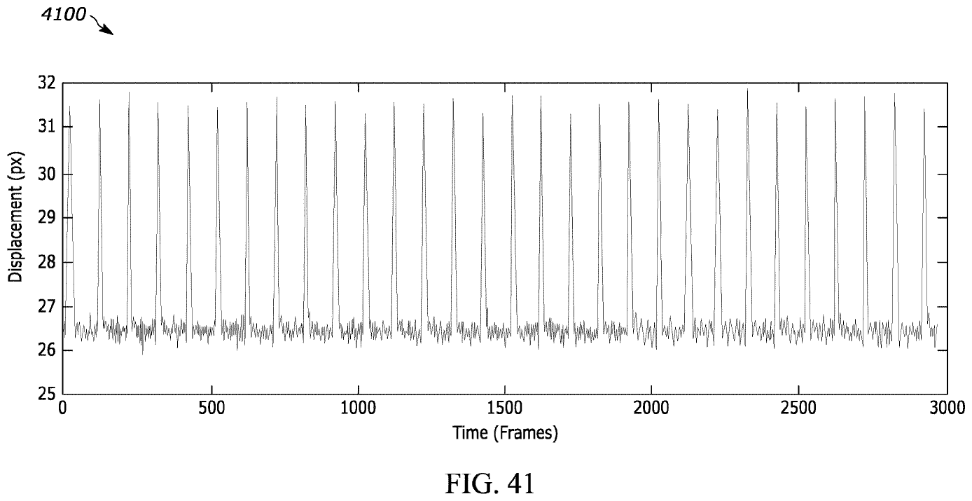

FIG. 41 shows a generated model corresponding to a time-series of scaffold deflection values according to an embodiment of the present disclosure.

FIG. 41 shows a plot 4100 of a model comprising a time-series of scaffold deflection values over a plurality of time points. In the plot 4100 shown in FIG. 41, the time-series of scaffold deflection values comprises a plurality of pixel-based distance, or displacement, values determined using the approaches described above. Each value within the time-series corresponds to a deflection value determined from a respective image of the plurality of images. As such, the x-axis of the plot 4100 corresponds to the plurality of time points (which corresponds to the plurality of frames or images) and the y-axis corresponds to the displacement of the tissue scaffold in pixels.

The skilled person will appreciate that, whilst the model show in the plot 4100 records the displacement values in pixels, a model may additionally or alternatively record displacement values in μm, areas (e.g., a count of total pixels or a measure in px² or μm²), and/or force. A displacement value in pixels may be converted to μm based on the resolution at which the image from which the displacement value is recorded was captured. Area-based scaffold deflection values may be estimated from the areas of the shapes bounded by the respective curves of the plurality of curves and the reference line (as described in more detail above in relation to FIG. 38). Force-based scaffold deflection values may be determined from the plurality of displacement values using a predetermined force displacement model. The predetermined force displacement model is operable to convert a displacement value in μm to a force-based displacement value (e.g., in μN). The predetermined force displacement model may be empirically generated and may be specific to the tissue scaffold used. In one implementation, the force displacement model is a third degree polynomial function of the form:

$${F}_{i} = 1.7 \times 1 {0}^{- 04} {δ}_{i}^{3} + 2.5 \times 1 {0}^{- 03} {δ}_{i}^{2} + 7 \times 1 {0}^{- 01} {δ}_{i} \qquad ( 1 )$$

Where δᵢ is the i-th displacement value of the plurality of displacement values.

As stated above, the model generated from the plurality of displacement values may alternatively comprise a force displacement model. The force displacement model is generated from a plurality of displacement values determined from a plurality of images capturing deflections of a flexible scaffold due to predetermined contractile forces exerted on the flexible scaffold over a predetermined plurality of time points (e.g., by a probe or other instrument).

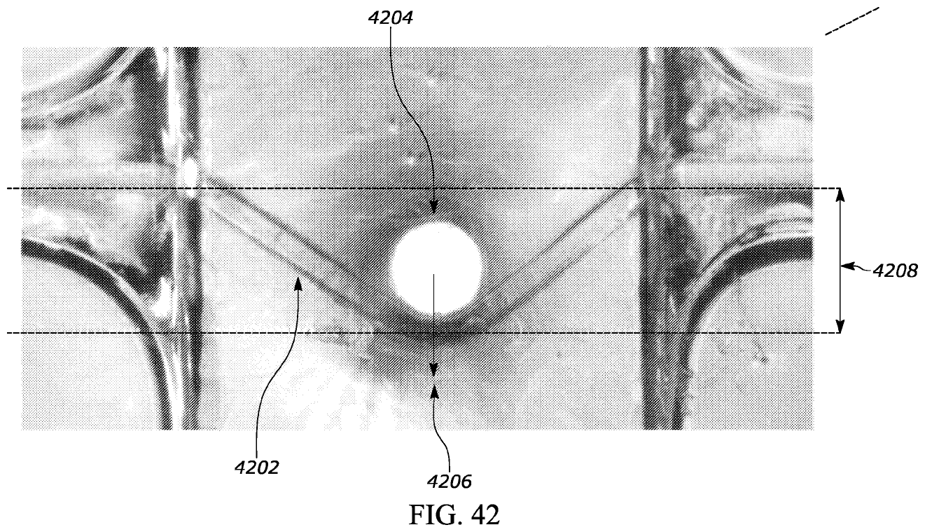

FIG. 42 shows a flexible scaffold deflecting due to a predetermined force exerted thereupon by a probe according to an embodiment of the present disclosure.

FIG. 42 shows a flexible scaffold 4202 and a probe 4204. The probe 4204 is exerting a predetermined force 4206 on the flexible scaffold 4202 resulting in a deflection 4208 of the flexible scaffold 4202 (i.e., a deflection from a resting position of the flexible scaffold 4202 to the deflected, or transformed, position shown in FIG. 42). Because the force exerted on the flexible scaffold 4202 by the probe 4204 is predetermined, or known, this force can be correlated to the deflection 4208 of the flexible scaffold 4202 caused by the predetermined force 4206. This correlation allows for a force-displacement model to be accurately and efficiently generated.

Multiple predetermined force values across different deflections of the flexible scaffold 4202 are obtained by moving the probe 4204 to a plurality of predetermined locations (displacements). The force exerted upon the flexible scaffold 4202 by the probe 4204 at each of the predetermined locations is measured using a force sensor (not shown). As stated above, the correlation between the (known) forces exerted upon the flexible scaffold 4202 by the probe 4204 at the plurality of predetermined locations and the resulting deflections of the flexible scaffold 4202 (determined using the above described approaches) are used to generate an accurate, scaffold specific, force-displacement model.

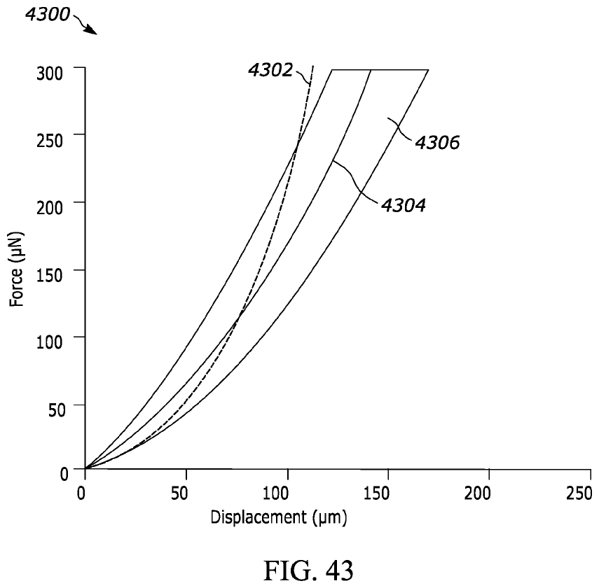

FIG. 43 shows a plot 4300 of force-displacement models according to embodiments of the present disclosure.

The plot 4300 comprises a first force-displacement model 4302, a second force-displacement model 4304, and a confidence interval 4306 associated with the second force-displacement model 4304. The first force-displacement model 4302 corresponds to a generic force-displacement model whilst the second force-displacement model 4304 is generated from empirical deflection values for a specific tissue scaffold. Both the first force-displacement model 4302 and the second force-displacement model 4304 are operable to estimate a force value (in μN) from a displacement, or deflection, value (in μm).

The second force-displacement model 4304 is generated by fitting a model to a plurality of predetermined forces and a plurality of displacement values generated from images of a flexible scaffold(s) deflecting in response to the predetermined forces. As described above, a plurality of images may be obtained whereby each image captures the deflection of a tissue scaffold at a respective time point. Because the force exerted on the tissue scaffold at a given time point is known, the (contractile) forces may be associated with each image and used to model the displacement or deflection of the tissue scaffold (as estimated using the approach described above) in relation to force. The force-displacement model (such as the second force-displacement model 4304) is generated by fitting a polynomial function to the plurality of predetermined forces and the plurality of displacement values. In one implementation, a third degree polynomial function is used. Alternatively, the force-displacement model is generated by training a machine learning model such as a multilayer perceptron, non-linear regression model, or the like on the plurality of predetermined forces and the plurality of displacement values.

Beneficially, generating a force-displacement model using the above approach allows for accurate force-displacement models to be used which are specific to a given device and/or tissue scaffold. That is, a force-displacement model may be generated for a specific device or tissue scaffold thereby allowing any variances in contractile responses of the tissue scaffold to be modelled within the force-displacement model thereby improving the consistency of force measurements obtained from displacement values provided to the model.

The description will now turn to methods for utilizing components, processes, and operations of the above described systems to model contractile deflection values of flexible tissue scaffolds.

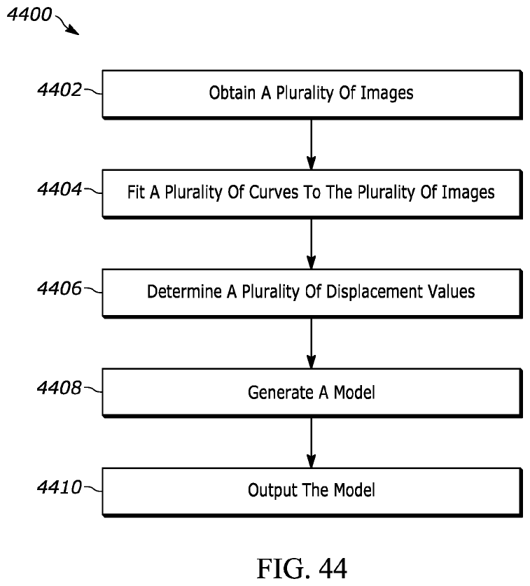

FIG. 44 shows a method 4400 for modelling contractile deflection values of flexible tissue scaffolds according to an aspect of the present disclosure.

The method 4400 comprises the steps of obtaining 4402 a plurality of images, fitting 4404 a plurality of curves to the plurality of images, determining 4406 a plurality of displacement values, and generating 4408 a model. The method 4400 further comprises the optional step of outputting 4410 the model. In one embodiment, the method 4400 is performed by the signal processing unit 112 of the system 100 of FIG. 1.

In general, the method 4400 generates a model which characterizes the (contractile) forces exerted on a flexible scaffold over a plurality of time points. Displacement values extracted from a plurality of images of the flexible scaffold over the plurality of time points are used to determine the model. Beneficially, models generated by the method 4400 may encode the contractile response of an engineered tissue thereby providing a quantification of a functional response of the engineered tissue. Alternatively, such models may be used to encode or calibrate the relationship between contractile force and measured displacement thereby improving the accuracy of contractile force measurements obtained from such models. Improvements to such models provide improvements to the downstream tasks which utilize such model whilst also improving the efficiency and performance of computing systems which are used to generate and deploy such models.

At the step of obtaining 4402, a plurality of images of a flexible scaffold at a plurality of time points are obtained. The plurality of images capture deflections of the flexible scaffold along a first dimension due to contractile forces exerted thereupon at the plurality of time points.

In one embodiment, the images are obtained from a device comprising the flexible scaffold and an imaging apparatus configured to obtain images of the flexible scaffold. The plurality of images may be grayscale bright-field images or fluorescence images. The deflections of the flexible scaffold captured in the plurality of images may be due to contractile forces exerted on the flexible scaffold by a tissue attached thereto (e.g., a natural tissue or an artificial tissue such as an engineered muscle-cardiac or skeletal-tissue) or due to a plurality of predetermined forces exerted on the flexible scaffold by a probe or other instrument at the plurality of time points.

At the step of fitting 4404, a plurality of curves are fit to the plurality of images such that each curve extends along a centerline of the flexible scaffold within a respective image of the plurality of images (e.g., the curve 3810 extends along the centerline of the flexible scaffold 3804 within the image region 3802 as described in relation to FIG. 38 above).

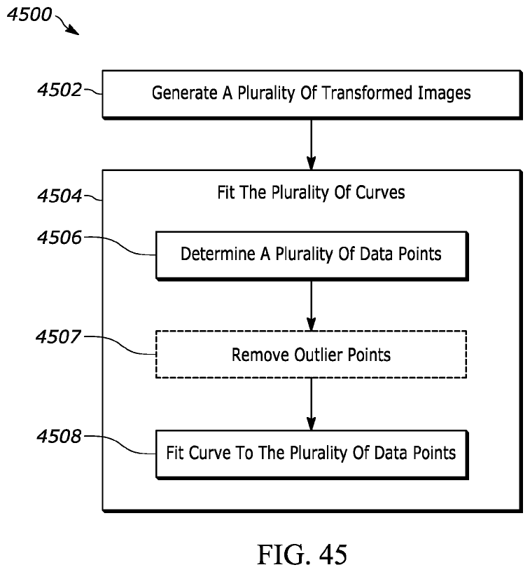

As will be described in more detail in relation to FIG. 45 below, in one embodiment the plurality of curves are fit to a plurality of transformed images generated from the plurality of images. More particularly, each curve of the plurality of curves is fit to a plurality of data points corresponding to likely locations of the centerline of the flexible scaffold within a respective transformed image.

At the step of determining 4406, a plurality of displacement values are determined from the plurality of curves. Each of the plurality of displacement values comprises a measurement along the first dimension between a respective curve of the plurality of curves and a reference line extending along a second dimension perpendicular to the first dimension.

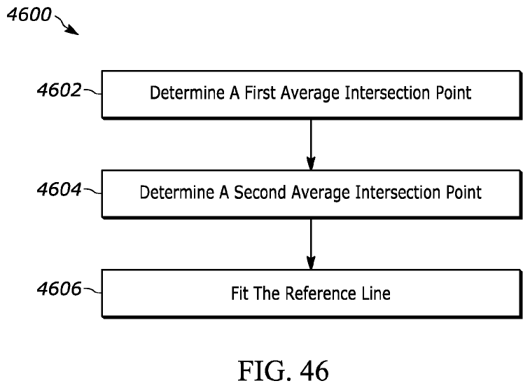

The reference line (e.g., the reference line 3820 shown in FIG. 38) comprises an expected position of the flexible scaffold when at rest. As will be described in more detail in relation to FIG. 46 below, in one embodiment the reference line is determined from a pair of average intersection points determined from the plurality of curves determined at the step of fitting 4404.

Each of the plurality of displacement values are determined at a reference point along the second dimension (e.g., the reference point C24 along the second dimension 408 shown in FIG. 38). In one embodiment, the reference point is determined such that an orthogonal distance, along the first dimension, between an average curve and the reference line is maximal at the reference point. The average curve is either the mean curve or median curve of the plurality of curves. In one embodiment, the reference point is replaced by a predetermined reference point if the reference point lies outside of a predetermined expected range. The predetermined expected range corresponds to the portion of the image, or image region, within which the reference point is expected to lie. That is, the portion of the image, or image region, which is expected to comprise the maximum orthogonal distance between the curve or average curve and the reference line. The predetermined expected range thus comprises a proportion of a length of the image, or image region, along the second dimension which is centralized along the second dimension. The proportion is from 20% to 80% of the length of the image, or image region, along the second dimension. In one implementation, the proportion is 60% of the length of the image, or image region, along the second dimension. The predetermined reference point used to replace the reference point if the reference point lies outside of the predetermined expected range may be the midpoint of the length of the image, or image region, along the second dimension.

At the step of generating 4408, a model is generated based on the plurality of displacement values. The model characterizes contractile forces exerted on the flexible scaffold over the plurality of time points.

In one embodiment, the generated model comprises a time-series of scaffold deflection values for the plurality of time points (e.g., the time-series shown in the plot 4100 of FIG. 41). The scaffold deflection values may be distance values, or pixel-based distance values, determined from the plurality of displacement values. Alternatively, the scaffold deflection values may be force values determined from the plurality of displacement values using a predetermined force-displacement model. In a further embodiment, the generated model comprises a force-displacement model operable to estimate a force value from an input scaffold displacement value (e.g., as illustrated in FIG. 43 and described in detail above).

At the optional step of outputting 4410, the model is output. In one embodiment, outputting the model comprises storing, or saving, the model to a persistent storage such as a non-volatile memory, a non-transitory medium, or the like. Additionally, or alternatively, outputting the model comprises transmitting the model via a network (e.g., a local area network, a wide area network, and the like), or displaying the model for review by a user.

FIG. 45 shows a method 4500 for fitting a plurality of curves according to an embodiment of the present disclosure.

In one embodiment, the method 4500 is performed as part of the step of fitting 4404 a plurality of curves in the method 4400 descried above in relation to FIG. 44. The method 4500 comprises the steps of generating 4502 a plurality of transformed images and fitting 4504 the plurality of curves. The step of fitting 4504 the plurality of curves comprises, for each image of the plurality of transformed images, the steps of determining 4506 a plurality of data points and fitting 4508 a curve to the plurality of data points. The step of fitting 4504 the plurality of curves further comprises the optional step of removing 4507 outlier points. In one embodiment, the method 4500 is performed by the signal processing unit 112 of the system 100 of FIG. 1.

At the step of generating 4502, a plurality of transformed images are generated from the plurality of images using a filtering operation. The filtering operation determines a likelihood that a region of an image contains the flexible scaffold.

The filtering operation determines a likelihood that a region or portion (i.e., a pixel or neighborhood of pixels) of an image contains a flexible scaffold. A transformed image thus comprises a transformed representation of an image where each pixel value in the transformed image corresponds to the likelihood that the pixel value lies on a flexible scaffold. The filtering operation comprises a vessel enhancement filter such as a multiscale vessel enhancement filter (e.g., a Hessian-based Frangi vesselness filter) or diffusion filtering (e.g., a coherence-enhancing diffusion filter). In an embodiment, the filtering operation comprise convolving a one-dimensional kernel across one dimension of the image as illustrated in FIG. 39.

At the step of fitting 4504, a plurality of curves are fit to the plurality of transformed images. That is, for each transformed image within the plurality of transformed image, a curve is fit according to the steps of determining 4506, optionally removing 4507, and fitting 4508.

At the step of determining 4506, a plurality of data points are determined for a transformed image of the plurality of transformed images. Each data point within the plurality of data points corresponds to a likely location along the centerline of the flexible scaffold within the first transformed image (e.g., as shown in FIG. 40 and described in more detail above).

At the optional step of removing 4507, one or more outlier points are removed from the plurality of data points. The one or more outlier points exceed a predetermined outlier threshold (e.g., as shown in FIG. 40 and described in more detail above).

The predetermined outlier threshold comprises a neighborhood distance threshold such that a distance between an outlier point of the one or more outlier points and each of the plurality of data points exceeds a predetermined distance threshold (e.g., the first outlier point 4008 shown in FIG. 40 is considered an outlier point because no other data points lie within the first neighborhood 4006 around the first outlier point 4008). Additionally, or alternatively, the predetermined outlier threshold comprises a pixel intensity threshold such that a pixel value at an image location corresponding to a location of an outlier point exceeds a predetermined intensity threshold. In one implementation, the predetermined intensity threshold is 3 times the median absolute deviation (MAD) of all pixel intensities for pixels lying along the tissue scaffold.

At the step of fitting 4508, a first curve of the plurality of curves is fit to the plurality of data points determined at the step of determining 4506 (e.g., the curve 4004 fit to the plurality of data points shown in FIG. 40).

In an embodiment, the curve is fit to the plurality of data points using a least squares approach such that the sum of squares of the residuals—the deviations between the plurality of data points and a set of corresponding data points on the curve—is minimized.

The curve fit at the step of fitting 4508 for a respective image, corresponds to the approximate centerline of the flexible scaffold captured within the respective image.

The steps of determining 4506, optionally removing 4507, and fitting 4508 are repeated for each transformed image within the plurality of transformed images such that the output of the step of fitting 4504 the plurality of curves comprises a plurality of curves where each curve extends along a centerline of the flexible scaffold within a respective transformed image of the plurality of transformed images.

FIG. 46 shows a method 4600 for fitting a reference line according to an embodiment of the present disclosure.

In one embodiment, the method 4600 is performed as part of the step of determining 4406 the plurality of displacement values in the method 4400 described above. The method 4600 comprises the steps of determining 4602 a first average intersection point, determining 4604 a second average intersection point, and fitting 4606 the reference line. In one embodiment, the method 4600 is performed by the signal processing unit 112 of the system 100 of FIG. 1.

At the step of determining 4602, a first average intersection point is determined between the plurality of curves and a first edge of an image region within the plurality of images.

In one embodiment, the first edge corresponds to a shared edge of the plurality of images (e.g., the top edge of each of the plurality of images). Alternatively, the first edge corresponds to an edge of a sub-region, or bounding box, of each of the plurality of images. An example first intersection point and first edge are shown by the first intersection point 3814 and the first edge 3812 in FIG. 38. The first average intersection point comprises the median point of intersection between the plurality of curves and the first edge. Alternatively, the average intersection point comprises the mean point of intersection.

At the step of determining 4604, a second average intersection point is determined between the plurality of curves and a second edge of the image region within the plurality of images.

In one embodiment, the second edge corresponds to a shared edge of the plurality of images (e.g., the bottom edge of each of the plurality of images). Alternatively, the second edge corresponds to an edge of a sub-region, or bounding box, of each of the plurality of images. An example second intersection point and second edge are shown by the second intersection point 3818 and the second edge 3816 in FIG. 38. The second average intersection point comprises the median point of intersection between the plurality of curves and the second edge. Alternatively, the average intersection point comprises the mean point of intersection.

At the step of fitting 4606, a reference line is fit such that the reference line extends between the first average intersection point and the second average intersection point. The reference line may be used to define a specific dimension (i.e., the second dimension 3808 shown in FIG. 38).

### Example Computing System and Components.

FIG. 47 shows an example computing system for carrying out the methods of the present disclosure. Specifically, FIG. 47 shows a block diagram of an embodiment of a computing system according to example embodiments of the present disclosure.

Computing system 4700 can be configured to perform any of the operations disclosed herein such as, for example, any of the operations discussed with reference to the functional modules described in relation to FIG. 1. Computing system includes one or more computing device(s) 4702. The one or more computing device(s) 4702 of computing system 4700 comprise one or more processors 4704 and memory 4706. One or more processors 4704 can be any general purpose processor(s) configured to execute a set of instructions, such as computing instructions including implemented in any one or more programming languages such as Python, Go, C, C++, C#, Java, or the like. For example, one or more processors 4704 can be one or more general-purpose processors, one or more field programmable gate array (FPGA), and/or one or more application specific integrated circuits (ASIC). In one embodiment, one or more processors 4704 include one processor. Alternatively, one or more processors 4704 include a plurality of processors that are operatively connected. One or more processors 4704 are communicatively coupled to memory 4706 via address bus 4708, control bus 4710, and data bus 4712.

Memory 4706 can be a random access memory (RAM), a read only memory (ROM), a persistent storage device such as a hard drive, an erasable programmable read only memory (EPROM), and/or the like. The one or more computing device(s) 4702 further comprise I/O interface 4714 communicatively coupled to address bus 4708, control bus 4710, and data bus 4712.

Memory 4706 can store information that can be accessed by one or more processors 4704. For instance, memory 4706 (e.g., one or more non-transitory computer-readable storage mediums, memory devices) can include computer-readable instructions (not shown) (e.g., computing instructions) that can be executed by one or more processors 4704. The computer-readable instructions can be software written in any suitable programming language or can be implemented in hardware. Additionally, or alternatively, the computer-readable instructions can be executed in logically and/or virtually separate threads on one or more processors 4704. For example, memory 4706 can store instructions (not shown), such as computing instructions, that when executed by one or more processors 4704 cause one or more processors 4704 to perform operations such as any of the operations and functions for which computing system 4700 is configured, as described herein. In addition, or alternatively, memory 4706 can store data (not shown), such as computing instructions, that can be obtained, received, accessed, written, manipulated, created, and/or stored. The data can include, for instance, the data and/or information described herein in relation to FIGS. 1 to 46. In some implementations, the one or more computing device(s) 4702 can obtain from and/or store data in one or more memory device(s) that are remote from the computing system 4700.

Computing system 4700 further comprises storage unit 4716, network interface 4718, input controller 4720, and output controller 4722. Storage unit 4716, network interface 4718, input controller 4720, and output controller 4722 are communicatively coupled to the central control unit (i.e., the memory 4706, the address bus 4708, the control bus 4710, and the data bus 4712) via I/O interface 4714.

Storage unit 4716 is a computer readable medium, preferably a non-transitory computer readable medium, comprising one or more programs, the one or more programs comprising computing instructions which when executed by the one or more processors 4704 cause computing system 4700 to perform the method steps of the present disclosure. Alternatively, storage unit 4716 is a transitory computer readable medium. Storage unit 4716 can be a persistent storage device such as a hard drive, a cloud storage device, or any other appropriate storage device.

Network interface 4718 can be a Wi-Fi module, a network interface card, a Bluetooth module, and/or any other suitable wired or wireless communication device. In an embodiment, network interface 4718 is configured to connect to a network such as a local area network (LAN), or a wide area network (WAN), the Internet, or an intranet.

### Example Aspects of the Disclosure

Clause 1. A method for processing a functional response waveform, the method comprising: obtaining, by one or more processors, a first waveform comprising at least one contraction response and at least one relaxation response of an engineered tissue, the first waveform having a predetermined length corresponding to an expected length of a contraction-relaxation cycle of the engineered tissue; determining, by the one or more processors, a predicted contraction type of a plurality of contraction types for the first waveform, wherein the plurality of contraction types include a single contraction type and a double contraction type; fitting, by the one or more processors, a model to the first waveform based on the predicted contraction type, wherein the model parameterizes growth of the at least one contraction response of the engineered tissue independently of growth of the at least one relaxation response of the engineered tissue; and generating, by the one or more processors, a second waveform from the model fit to the first waveform such that the second waveform comprises a noise filtered representation of the first waveform.

Clause 2. The method of any one of the preceding clauses further comprising: extracting, by the one or more processors, one or more feature values from the second waveform.

Clause 3. The method of clause 2 further comprising: outputting, by the one or more processors, the one or more feature values extracted from the second waveform.

Clause 4. The method of clause 2 wherein the one or more feature values include one or more of at least one contraction-relaxation cycle amplitude value, at least one contraction time value, at least one maximum contraction slope value, at least one relaxation time value, at least one maximum relaxation slope value, and at least one contraction-relaxation cycle duration value.

Clause 5. The method of any one of the preceding clauses wherein the first waveform comprises a first contraction response and a first relaxation response of the engineered tissue.

Clause 6. The method of clause 5 wherein the predicted contraction type is the single contraction type.

Clause 7. The method of clause 5 wherein the step of fitting the model to the first waveform comprises fitting a first model to the first waveform, wherein the first model parameterizes growth of the first contraction response independently of growth of the first relaxation response.

Clause 8. The method of clause 7 wherein the first model comprises a first function having a positive growth rate and a second function having a negative growth rate.

Clause 9. The method of clause 8 wherein the first contraction response is modeled by the first function.

Clause 10. The method of clause 8 wherein the first relaxation response is modeled by the second function.

Clause 11. The method of clause 8 wherein the first model comprises a product of the first function and the second function.

Clause 12. The method of clause 8 wherein the first function and the second function are logistic functions.

Clause 13. The method of clause 8 wherein the first function and the second function are biexponential functions.

Clause 12. The method of clause 7 wherein the first waveform comprises a second contraction response and a second relaxation response of the engineered tissue.

Clause 13. The method of clause 12 wherein the predicted contraction type is the double contraction type.

Clause 14. The method of clause 12 wherein the step of fitting the model to the first waveform comprises fitting a second model to the second waveform, wherein the second model parameterizes growth of the second contraction response independently of growth of the second relaxation response.

Clause 15. The method of clause 14 wherein the model comprises a combination of the first model and the second model.

Clause 16. The method of clause 14 wherein the second model comprises a third function having a positive growth rate and a fourth function having a negative growth rate.

Clause 17. The method of clause 16 wherein the second contraction response is modeled by the third function.

Clause 18. The method of clause 16 wherein the second relaxation response is modeled by the fourth function.

Clause 19. The method of clause 16 wherein the second model comprises a product of the third function and the fourth function.

Clause 20. The method of clause 16 wherein the third function and the fourth function are logistic functions.

Clause 21. The method of clause 16 wherein the third function and the fourth function are biexponential functions.

Clause 20. The method of any one of the preceding clauses wherein the model comprises a plurality of parameters associated with the at least one contraction response and the at least one relaxation response.

Clause 21. The method of clause 20 wherein the plurality of parameters comprise at least one maximum value parameter, at least one rate of rise parameter, at least one rate of fall parameter, a y-shift parameter, at least one rising x-shift parameter, and at least one falling x-shift parameter.

Clause 22. The method of clause 20 wherein the step of fitting the model to the first waveform comprises: predicting, by the one or more processors, a plurality of values for the plurality of parameters of the model such that the model fit to the first waveform comprises the plurality of values for the plurality of parameters.

Clause 23. The method of clause 22 wherein the plurality of values is predicted using a machine learning model.

Clause 24. The method of clause 23 wherein the machine learning model comprises a trained inception model.

Clause 25. The method of clause 23 wherein the machine learning model comprises a trained convolutional neural network with dilated convolution.

Clause 26. The method of clause 23 wherein the machine learning model comprises a trained long short-term memory neural network.

Clause 27. The method of clause 22 wherein the step of fitting the model to the first waveform further comprises: optimizing, by the one or more processors, the plurality of values by minimizing an error between the first waveform and the model fit to the first waveform using the plurality of values for the plurality of parameters of the model.

Clause 28. The method of clause 27 wherein the plurality of values is used as starting values for the step of optimizing.

Clause 28. The method of clause 27 wherein the plurality of values is optimized using a simplex search algorithm.

Clause 29. The method of any one of the preceding clauses wherein the predetermined length is from 0.05 s to 10 s.

Clause 30. The method of clause 29 wherein the predetermined length is from 0.15 s to 1 s.

Clause 31. The method of any one of the preceding clauses wherein the at least one contraction response and the at least one relaxation response correspond to a functional response of the engineered tissue stimulated at a first frequency.

Clause 32. The method of clause 31 wherein the predetermined length is proportional to the first frequency.

Clause 33. The method of any one of the preceding clauses wherein the predicted contraction type is determined by a classifier based on the first waveform input to the classifier.

Clause 34. The method of clause 33 wherein the classifier comprises a trained neural network.

Clause 35. The method of any one of the preceding clauses wherein the first waveform is obtained from a waveform comprising a plurality of contraction-relaxation cycles of the engineered tissue.

Clause 36. The method of any one of the preceding clauses wherein the first waveform is obtained from a bioreactor comprising the engineered tissue.

Clause 37. The method of any one of the preceding clauses wherein the engineered tissue comprises engineered muscle tissue.

Clause 38. The method of clause 37 wherein the engineered muscle tissue is engineered cardiac tissue.

Clause 39. The method of clause 37 wherein the engineered muscle tissue is engineered skeletal muscle tissue.

Clause 40. A non-transitory computer readable medium storing computing instructions which, when executed by a device comprising one or more processors, cause the one or more processors to: obtain a first waveform comprising at least one contraction response and at least one relaxation response of an engineered tissue, the first waveform having a predetermined length corresponding to an expected length of a contraction-relaxation cycle of the engineered tissue; determine a predicted contraction type of a plurality of contraction types for the first waveform, wherein the plurality of contraction types include a single contraction type and a double contraction type; fit a model to the first waveform based on the predicted contraction type, wherein the model parameterizes growth of the at least one contraction response of the engineered tissue independently of growth of the at least one relaxation response of the engineered tissue; and generate a second waveform from the model fit to the first waveform such that the second waveform comprises a noise filtered representation of the first waveform.

Clause 41. A system comprising: one or more processors; and a memory storing computing instructions which, when executed by the one or more processors, cause the one or more processors to: obtain a first waveform comprising at least one contraction response and at least one relaxation response of an engineered tissue, the first waveform having a predetermined length corresponding to an expected length of a contraction-relaxation cycle of the engineered tissue; determine a predicted contraction type of a plurality of contraction types for the first waveform, wherein the plurality of contraction types include a single contraction type and a double contraction type; fit a model to the first waveform based on the predicted contraction type, wherein the model parameterizes growth of the at least one contraction response of the engineered tissue independently of growth of the at least one relaxation response of the engineered tissue; and generate a second waveform from the model fit to the first waveform such that the second waveform comprises a noise filtered representation of the first waveform.

Clause 42. A method for training a classifier to predict a tissue contraction type from a functional response waveform, the method comprising: obtaining, by one or more processors, a plurality of waveforms comprising functional responses of a one or more engineered tissues, each of the plurality of waveforms having a predetermined length corresponding to an expected length of a contraction-relaxation cycle of an engineered tissue; extracting, by the one or more processors, a first plurality of parameter sets from a first subset of the plurality of waveforms associated with a single contraction type, wherein a first parameter set of the first plurality of parameter sets characterizes a first waveform of the first subset of waveforms; extracting, by the one or more processors, a second plurality of parameter sets from a second subset of the plurality of waveforms associated with a double contraction type, wherein a second parameter set of the second plurality of parameter sets characterizes a second waveform of the second subset of waveforms; determining, by the one or more processors, a plurality of parameter set distributions, wherein the plurality of parameter set distributions comprise a first parameter set distribution determined from the first plurality of parameter sets and a second parameter set distribution determined from the second plurality of parameter sets; generating, by the one or more processors, a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and training, by the one or more processors, the classifier using the synthetic training data set, wherein the classifier trained using the synthetic training data set determines a predicted tissue contraction type for an input waveform.

Clause 43. The method of clause 42 wherein the classifier comprises a machine learning model.

Clause 44. The method of clause 43 wherein the machine learning model comprises a convolutional neural network.

Clause 45. The method of any one of clauses 42-44 further comprising: obtaining, by the one or more processors, a first waveform comprising a functional response of a first artificial tissue, the first waveform having a length corresponding to an expected length of a contraction-relaxation cycle of the first artificial tissue; and predicting, by the one or more processors and using the classifier trained on the synthetic training data set, a tissue contraction type from the first waveform.

Clause 46. The method of clause 45 further comprising: outputting, by the one or more processors, the tissue contraction type predicted using the classifier.

Clause 47. The method of any one of clauses 42-46 wherein the first parameter set comprises a first plurality of parameter values of a first model, wherein the first model comprises a rising logistic function having a positive growth rate and a falling logistic function having a negative growth rate.

Clause 48. The method of clause 47 wherein the first plurality of parameter values comprises a maximum value parameter value, a rate of rise parameter value, a rate of fall parameter value, a y-shift parameter value, a rising x-shift parameter value, and a falling x-shift parameter value.

Clause 49. The method of any one of clauses 42-48 wherein the second parameter set comprises a second plurality of parameter values of a second model, wherein the second model comprises a plurality of rising logistic functions having positive growth rates and a plurality of first falling logistic function having negative growth rates.

Clause 50. The method of clause 49 wherein the second plurality of parameter values comprise a plurality of maximum value parameter values, a plurality of rate of rise parameter values, a plurality of rate of fall parameter values, a plurality of y-shift parameter values, a plurality of rising x-shift parameter values, and a plurality of falling x-shift parameter values.

Clause 51. The method of any one of clauses 42-50 wherein the one or more engineered tissues comprise one or more engineered muscle tissues.

Clause 52. The method of clause 51 wherein the one or more engineered muscle tissues are engineered cardiac tissues.

Clause 53. The method of clause 51 wherein the one or more engineered muscle tissues are engineered skeletal muscle tissues.

Clause 54. A non-transitory computer readable medium storing computing instructions which, when executed by a device comprising one or more processors, cause the one or more processors to: obtain a plurality of waveforms comprising functional responses of a one or more engineered tissues, each of the plurality of waveforms having a predetermined length corresponding to an expected length of a contraction-relaxation cycle of an engineered tissue; extract a first plurality of parameter sets from a first subset of the plurality of waveforms associated with a single contraction type, wherein a first parameter set of the first plurality of parameter sets characterizes a first waveform of the first subset of waveforms; extract a second plurality of parameter sets from a second subset of the plurality of waveforms associated with a double contraction type, wherein a second parameter set of the second plurality of parameter sets characterizes a second waveform of the second subset of waveforms; determine a plurality of parameter set distributions, wherein the plurality of parameter set distributions comprise a first parameter set distribution determined from the first plurality of parameter sets and a second parameter set distribution determined from the second plurality of parameter sets; generate a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and train a classifier using the synthetic training data set, wherein the classifier trained using the synthetic training data set determines a predicted tissue contraction type for an input waveform.

Clause 55. A system comprising: one or more processors; and a memory storing computing instructions which, when executed by the one or more processors, cause the one or more processors to: obtain a plurality of waveforms comprising functional responses of a one or more engineered tissues, each of the plurality of waveforms having a predetermined length corresponding to an expected length of a contraction-relaxation cycle of an engineered tissue; extract a first plurality of parameter sets from a first subset of the plurality of waveforms associated with a single contraction type, wherein a first parameter set of the first plurality of parameter sets characterizes a first waveform of the first subset of waveforms; extract a second plurality of parameter sets from a second subset of the plurality of waveforms associated with a double contraction type, wherein a second parameter set of the second plurality of parameter sets characterizes a second waveform of the second subset of waveforms; determine a plurality of parameter set distributions, wherein the plurality of parameter set distributions comprise a first parameter set distribution determined from the first plurality of parameter sets and a second parameter set distribution determined from the second plurality of parameter sets; generate a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and train a classifier using the synthetic training data set, wherein the classifier trained using the synthetic training data set determines a predicted tissue contraction type for an input waveform.

Clause 56. A method for processing a functional response waveform the method comprising: obtaining, by one or more processors, a first waveform comprising a plurality of functional responses of an artificial tissue stimulated at a first frequency; convolving, by the one or more processors, the first waveform with a pulse-train to generate a convolved waveform, wherein the pulse-train is generated at the first frequency; identifying, by the one or more processors, a first location associated with a first maximum value of the convolved waveform, wherein the first location corresponds to an expected location of a first contraction-relaxation cycle; and extracting, by the one or more processors and from the first location of the first waveform, a second waveform comprising the first contraction-relaxation cycle, wherein the second waveform has a first duration proportional to the first frequency.

Clause 57. The method of clause 56 further comprising: outputting, by the one or more processors, the second waveform.

Clause 58. The method of clause 57 further comprising: identifying, by the one or more processors, a second location associated with a second maximum value of the convolved waveform, wherein the second location corresponds to an expected location of a second contraction-relaxation cycle; and extracting, by the one or more processors and from the second location of the first waveform, a third waveform comprising the second contraction-relaxation cycle, wherein the second waveform has a second duration proportional to the first frequency.

Clause 59. The method of clause 58 further comprising: outputting, by the one or more processors, the third waveform.

Clause 60. The method of clause 58 wherein the first duration and the second duration are the same.

Clause 61. The method of any one of clauses 56-60 further comprising: generating, by the one or more processors, the pulse-train at the first frequency.

Clause 62. The method of any one of clauses 56-61 wherein a midpoint of the second waveform is substantially aligned to the first location of the first waveform.

Clause 63. The method of any one of clauses 56-62 wherein the first frequency is from 0.1 Hz to 20 Hz.

Clause 64. The method of clause 63 wherein the first frequency is from 1 Hz to 6 Hz.

Clause 65. The method of any one of clauses 56-64 wherein the first waveform is obtained from a bioreactor comprising the artificial tissue.

Clause 66. The method of any one of clauses 56-65 wherein the artificial tissue comprises engineered muscle tissue.

Clause 67. The method of clause 66 wherein the engineered muscle tissue is engineered cardiac tissue.

Clause 68. The method of clause 67 wherein the engineered muscle tissue is engineered skeletal muscle tissue.

Clause 69. A non-transitory computer readable medium storing computing instructions which, when executed by a device comprising one or more processors, cause the one or more processors to: obtain a first waveform comprising a plurality of functional responses of an artificial tissue stimulated at a first frequency; convolve the first waveform with a pulse-train to generate a convolved waveform, wherein the pulse-train is generated at the first frequency; identify a first location associated with a first maximum value of the convolved waveform, wherein the first location corresponds to an expected location of a first contraction-relaxation cycle; and extract, from the first location of the first waveform, a second waveform comprising the first contraction-relaxation cycle, wherein the second waveform has a first duration proportional to the first frequency.

Clause 70. A system comprising: one or more processors; and a memory storing computing instructions which, when executed by the one or more processors, cause the one or more processors to: obtain a first waveform comprising a plurality of functional responses of an artificial tissue stimulated at a first frequency; convolve the first waveform with a pulse-train to generate a convolved waveform, wherein the pulse-train is generated at the first frequency; identify a first location associated with a first maximum value of the convolved waveform, wherein the first location corresponds to an expected location of a first contraction-relaxation cycle; and extract, from the first location of the first waveform, a second waveform comprising the first contraction-relaxation cycle, wherein the second waveform has a first duration proportional to the first frequency.

Clause 71. A method for predicting a perturbation effect, the method comprising: obtaining, by one or more processors, a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions; splitting, by the one or more processors, the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue; determining, by the one or more processors, a predicted contraction type of a plurality of contraction types for each of the first plurality of waveforms, wherein the plurality of contraction types include a single contraction type and a double contraction type; fitting, by the one or more processors, a model to each waveform of the first plurality of waveforms based on a corresponding predicted contraction type, wherein the model parameterizes growth of the at least one contraction response independently of growth of the at least one relaxation response of the engineered tissue; generating, by the one or more processors, a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal; extracting, by the one or more processors, a first feature value of a first feature from the plurality of filtered baseline waveforms; extracting, by the one or more processors, a second feature value of the first feature from the plurality of filtered perturbation waveforms; and determining, by the one or more processors, an effect associated with the first set of perturbation conditions based on a comparison of the first feature value and the second feature value.

Clause 72. The method of clause 71 wherein the first feature is one of a total number of single contraction type waveforms and a total number of double contraction type waveforms.

Clause 73. The method of any one of clauses 71-72 wherein the first feature is one of a contraction-relaxation cycle amplitude, a contraction time, a maximum contraction slope, a relaxation time, a maximum relaxation slope, and a contraction-relaxation cycle duration.

Clause 74. The method of clause 73 wherein the first feature value comprises an average value of the first feature determined from the plurality of filtered baseline waveforms.

Clause 75. The method of clause 73 wherein the second feature value comprises an average value of the first feature determined from the plurality of filtered perturbation waveforms.

Clause 76. The method of any one of clauses 71-75 further comprising: outputting, by the one or more processors, the effect associated with a first compound.

Clause 77. The method of any one of clauses 71-76 wherein the step of fitting the model comprises fitting a first model for waveforms associated with the single contraction type.

Clause 78. The method of clause 77 wherein the first model comprises a first rising logistic function having a positive growth rate and a first falling logistic function having a negative growth rate.

Clause 79. The method of clause 78 wherein the first model comprises a product of the first rising logistic function and the first falling logistic function.

Clause 80. The method of clause 77 wherein the step of fitting the model comprises fitting the first model and a second model for waveforms associated with the double contraction type

Clause 81. The method of clause 80 wherein the second model comprises a second rising logistic function having a positive growth rate and a second falling logistic function having a negative growth rate.

Clause 82. The method of clause 81 wherein the second model comprises a product of the second rising logistic function and the second falling logistic function.

Clause 83. The method of any one of clauses 71-82 wherein the model comprises a plurality of parameters associated with the at least one contraction response and the at least one relaxation response.

Clause 84. The method of clause 83 wherein the plurality of parameters comprise at least one maximum value parameter, at least one rate of rise parameter, at least one rate of fall parameter, at least one y-shift parameter, at least one rising x-shift parameter, and at least one falling x-shift parameter.

Clause 85. The method of clause 83 wherein the step of fitting the model to a first waveform of the first plurality of waveforms comprises: predicting, by the one or more processors, a plurality of values for the plurality of parameters of the model such that the model fit to the first waveform comprises the plurality of values for the plurality of parameters.

Clause 86. The method of clause 85 wherein the plurality of values are predicted using a machine learning model.

Clause 87. The method of clause 86 wherein the machine learning model comprises a trained inception model.

Clause 88. The method of clause 86 wherein the machine learning model comprises a trained convolutional neural network with dilated convolution.

Clause 89. The method of clause 86 wherein the machine learning model comprises a trained long short-term memory neural network.

Clause 90. The method of clause 85 wherein the step of fitting the model to the first waveform further comprises: optimizing, by the one or more processors, the plurality of values by minimizing an error between the first waveform and the model fit to the first waveform using the plurality of values for the plurality of parameters of the model.

Clause 91. The method of clause 90 wherein the plurality of values are optimized using a simplex search algorithm.

Clause 92. The method of any one of clauses 71-91 wherein the predetermined length is from 0.05 s to 10 s.

Clause 93. The method of clause 92 wherein the predetermined length is from 0.15 s to 1 s.

Clause 94. The method of any one of clauses 71-93 wherein the at least one contraction response and the at least one relaxation response correspond to a functional response of the engineered tissue stimulated at a first frequency.

Clause 95. The method of clause 94 wherein the predetermined length is proportional to the first frequency.

Clause 96. The method of any one of clauses 71-95 wherein the predicted contraction type is determined by a classifier.

Clause 97. The method of clause 96 wherein the classifier comprises a trained convolutional neural network.

Clause 98. The method of any one of clauses 71-97 wherein the plurality of signals are obtained from a bioreactor comprising the engineered tissue.

Clause 99. The method of any one of clauses 71-98 wherein the engineered tissue comprises engineered muscle tissue.

Clause 100. The method of clause 99 wherein the engineered muscle tissue is engineered cardiac tissue.

Clause 101. The method of clause 99 wherein the engineered muscle tissue is engineered skeletal muscle tissue.

Clause 102. The method of any one of clauses 71-101 wherein the control conditions are associated with the engineered tissue having been vehicle treated.

Clause 103. The method of any one of clauses 71-102 wherein the first set of perturbation conditions are associated with one or more of: a treatment of the engineered tissue with a drug or compound; a different cell line; a disease state of the engineered tissue; a physical perturbation of the engineered tissue; and changes to an environment of the engineered tissue.

Clause 104. A non-transitory computer readable medium storing computing instructions which, when executed by a device comprising one or more processors, cause the one or more processors to: obtain a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions; split the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue; determine a predicted contraction type of a plurality of contraction types for each of the first plurality of waveforms, wherein the plurality of contraction types include a single contraction type and a double contraction type; fit a model to each waveform of the first plurality of waveforms based on a corresponding predicted contraction type, wherein the model parameterizes growth of the at least one contraction response independently of growth of the at least one relaxation response of the engineered tissue; generate a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal; extract a first feature value of a first feature from the plurality of filtered baseline waveforms; extract a second feature value of the first feature from the plurality of filtered perturbation waveforms; and determine an effect associated with the first set of perturbation conditions based on a comparison of the first feature value and the second feature value.

Clause 105. A system comprising: one or more processors; and a memory storing computing instructions which, when executed by the one or more processors, cause the one or more processors to: obtain a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions; split the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue; determine a predicted contraction type of a plurality of contraction types for each of the first plurality of waveforms, wherein the plurality of contraction types include a single contraction type and a double contraction type; fit a model to each waveform of the first plurality of waveforms based on a corresponding predicted contraction type, wherein the model parameterizes growth of the at least one contraction response independently of growth of the at least one relaxation response of the engineered tissue; generate a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal; extract a first feature value of a first feature from the plurality of filtered baseline waveforms; extract a second feature value of the first feature from the plurality of filtered perturbation waveforms; and determine an effect associated with the first set of perturbation conditions based on a comparison of the first feature value and the second feature value.

Clause 106. A method for training a model using synthetic training data, the method comprising: obtaining, by one or more processors, a plurality of waveforms comprising functional responses of one or more artificial tissues, wherein the plurality of waveforms all comprise a common contraction type of a predetermined plurality of contraction types; each of the plurality of waveforms having a predetermined length corresponding to an expected length of a contraction-relaxation cycle of an artificial tissue, of the plurality of waveforms have a same contraction type; extracting, by the one or more processors, a plurality of parameter sets from the plurality of waveforms, wherein a parameter set of the plurality of parameter sets characterizes a corresponding waveform of the plurality of waveforms; determining, by the one or more processors, a parameter set distribution from the plurality of parameter sets; generating, by the one or more processors, a synthetic training data set, each element of the synthetic training data set comprising a synthetic waveform and a corresponding parameter set used to generate the synthetic waveform, wherein the corresponding parameter set is obtained from the parameter set distribution; and training, by the one or more processors, a parameter estimation model using the synthetic training data set, wherein the parameter estimation model is trained to estimate an output parameter set from an input waveform.

Clause 107. The method of clause 106 wherein the parameter estimation model comprises an inception model.

Clause 108. The method of any one of clauses 106-107 wherein the parameter estimation model comprises a convolutional neural network with dilated convolution.

Clause 109. The method of any one of clauses 106-108 wherein the parameter estimation model comprises a long short-term memory neural network.

Clause 110. The method of any one of clauses 106-109 further comprising: obtaining, by the one or more processors, a first waveform comprising a functional response of a first artificial tissue; and predicting, by the one or more processors and using the parameter estimation model trained on the synthetic training data set, a first set of parameter values from the first waveform.

Clause 111. The method of clause 110 further comprising: outputting, by the one or more processors, the first set of parameter values.

Clause 112. The method of any one of clauses 106-111 wherein the step of generating the synthetic training data set further comprises: adding, by the one or more processors, a noise component to the synthetic waveform of each element of the synthetic training data set according to a noise model.

Clause 113. The method of clause 112 wherein the noise model is determined from the plurality of waveforms.

Clause 114. The method of any one of clauses 106-113 wherein the parameter set comprises parameter values of a model comprising at least one rising logistic function having a positive growth rate and at least one falling logistic function having a negative growth rate.

Clause 115. The method of clause 114 wherein the parameter values comprise at least one maximum value parameter value, at least one rate of rise parameter value, at least one rate of fall parameter value, a y-shift parameter value, at least one rising x-shift parameter value, and at least one falling x-shift parameter value.

Clause 116. The method of any one of clauses 106-115 wherein the one or more artificial tissues comprise one or more engineered muscle tissues.

Clause 117. The method of clause 116 wherein the one or more engineered muscle tissues are engineered cardiac tissues.

Clause 118. The method of clause 116 wherein the one or more engineered muscle tissues are engineered skeletal muscle tissues.

Clause 119. The method of any one of clauses 106-118 wherein the predetermined plurality of contraction types include a single contraction type and a double contraction type.

Clause 120. A non-transitory computer readable medium storing computing instructions which, when executed by a device comprising one or more processors, cause the one or more processors to: obtain a plurality of waveforms comprising functional responses of one or more artificial tissues during a single contraction-relaxation cycle; extract a plurality of parameter sets from the plurality of waveforms, wherein a parameter set of the plurality of parameter sets characterizes a corresponding waveform of the plurality of waveforms; determine a parameter set distribution from the plurality of parameter sets; generate a synthetic training data set, each element of the synthetic training data set comprising a synthetic waveform and a corresponding parameter set used to generate the synthetic waveform, wherein the corresponding parameter set is obtained from the parameter set distribution; and train a parameter estimation model using the synthetic training data set, wherein the parameter estimation model is trained to estimate an output parameter set from an input waveform.

Clause 121. A system comprising: one or more processors; and a memory storing computing instructions which, when executed by the one or more processors, cause the one or more processors to: obtain a plurality of waveforms comprising functional responses of one or more artificial tissues during a single contraction-relaxation cycle; extract a plurality of parameter sets from the plurality of waveforms, wherein a parameter set of the plurality of parameter sets characterizes a corresponding waveform of the plurality of waveforms; determine a parameter set distribution from the plurality of parameter sets; generate a synthetic training data set, each element of the synthetic training data set comprising a synthetic waveform and a corresponding parameter set used to generate the synthetic waveform, wherein the corresponding parameter set is obtained from the parameter set distribution; and train a parameter estimation model using the synthetic training data set, wherein the parameter estimation model is trained to estimate an output parameter set from an input waveform.

1. A method for processing a functional response waveform, the method comprising:
obtaining, by one or more processors, a first waveform comprising at least one contraction response and at least one relaxation response of an engineered tissue, the first waveform having a predetermined length corresponding to an expected length of a contraction-relaxation cycle of the engineered tissue;
determining, by the one or more processors, a predicted contraction type of a plurality of contraction types for the first waveform, wherein the plurality of contraction types include a single contraction type and a double contraction type;
fitting, by the one or more processors, a model to the first waveform based on the predicted contraction type, wherein the model parameterizes growth of the at least one contraction response of the engineered tissue independently of growth of the at least one relaxation response of the engineered tissue; and
generating, by the one or more processors, a second waveform from the model fit to the first waveform such that the second waveform comprises a noise filtered representation of the first waveform.

2. The method of claim 1 further comprising:
extracting, by the one or more processors, one or more feature values from the second waveform.

3. The method of claim 2 further comprising:
outputting, by the one or more processors, the one or more feature values extracted from the second waveform.

4. The method of claim 2 wherein the one or more feature values include one or more of at least one contraction-relaxation cycle amplitude value, at least one contraction time value, at least one maximum contraction slope value, at least one relaxation time value, at least one maximum relaxation slope value, and at least one contraction-relaxation cycle duration value.

5. The method of claim 1 wherein the first waveform comprises a first contraction response and a first relaxation response of the engineered tissue.

6. The method of claim 5 wherein the predicted contraction type is the single contraction type.

7. The method of claim 5 wherein the step of fitting the model to the first waveform comprises fitting a first model to the first waveform, wherein the first model parameterizes growth of the first contraction response independently of growth of the first relaxation response.

8. The method of claim 7 wherein the first model comprises a first function having a positive growth rate and a second function having a negative growth rate.

9. The method of claim 8 wherein the first contraction response is modeled by the first function.

10. The method of claim 8 wherein the first relaxation response is modeled by the second function.

11. The method of claim 8 wherein the first model comprises a product of the first function and the second function.

12. The method of claim 8 wherein the first function and the second function are logistic functions.

13. The method of claim 8 wherein the first function and the second function are biexponential functions.

12. The method of claim 7 wherein the first waveform comprises a second contraction response and a second relaxation response of the engineered tissue.

13. The method of claim 12 wherein the predicted contraction type is the double contraction type.

14. The method of claim 12 wherein the step of fitting the model to the first waveform comprises fitting a second model to the second waveform, wherein the second model parameterizes growth of the second contraction response independently of growth of the second relaxation response.

15. The method of claim 14 wherein the model comprises a combination of the first model and the second model.

16. The method of claim 14 wherein the second model comprises a third function having a positive growth rate and a fourth function having a negative growth rate.

17. The method of claim 16 wherein the second contraction response is modeled by the third function.

18. The method of claim 16 wherein the second relaxation response is modeled by the fourth function.

19. The method of claim 16 wherein the second model comprises a product of the third function and the fourth function.

20. The method of claim 16 wherein the third function and the fourth function are logistic functions.

21. The method of claim 16 wherein the third function and the fourth function are biexponential functions.

20. The method of claim 1 wherein the model comprises a plurality of parameters associated with the at least one contraction response and the at least one relaxation response.

21. The method of claim 20 wherein the plurality of parameters comprise at least one maximum value parameter, at least one rate of rise parameter, at least one rate of fall parameter, a y-shift parameter, at least one rising x-shift parameter, and at least one falling x-shift parameter.

22. The method of claim 20 wherein the step of fitting the model to the first waveform comprises:
predicting, by the one or more processors, a plurality of values for the plurality of parameters of the model such that the model fit to the first waveform comprises the plurality of values for the plurality of parameters.

23. The method of claim 22 wherein the plurality of values is predicted using a machine learning model.

24. The method of claim 23 wherein the machine learning model comprises a trained inception model.

25. The method of claim 23 wherein the machine learning model comprises a trained convolutional neural network with dilated convolution.

26. The method of claim 23 wherein the machine learning model comprises a trained long short-term memory neural network.

27. The method of claim 22 wherein the step of fitting the model to the first waveform further comprises:
optimizing, by the one or more processors, the plurality of values by minimizing an error between the first waveform and the model fit to the first waveform using the plurality of values for the plurality of parameters of the model.

28. The method of claim 27 wherein the plurality of values is used as starting values for the step of optimizing.

28. The method of claim 27 wherein the plurality of values is optimized using a simplex search algorithm.

29. The method of claim 1 wherein the predetermined length is from 0.05 s to 10 s.

30. The method of claim 29 wherein the predetermined length is from 0.15 s to 1 s.

31. The method of claim 1 wherein the at least one contraction response and the at least one relaxation response correspond to a functional response of the engineered tissue stimulated at a first frequency.

32. The method of claim 31 wherein the predetermined length is proportional to the first frequency.

33. The method of claim 1 wherein the predicted contraction type is determined by a classifier based on the first waveform input to the classifier.

34. The method of claim 33 wherein the classifier comprises a trained neural network.

35. The method of claim 1 wherein the first waveform is obtained from a waveform comprising a plurality of contraction-relaxation cycles of the engineered tissue.

36. The method of claim 1 wherein the first waveform is obtained from a bioreactor comprising the engineered tissue.

37. The method of claim 1 wherein the engineered tissue comprises engineered muscle tissue.

38. The method of claim 37 wherein the engineered muscle tissue is engineered cardiac tissue.

39. The method of claim 37 wherein the engineered muscle tissue is engineered skeletal muscle tissue.

40. A non-transitory computer readable medium storing computing instructions which, when executed by a device comprising one or more processors, cause the one or more processors to:
obtain a first waveform comprising at least one contraction response and at least one relaxation response of an engineered tissue, the first waveform having a predetermined length corresponding to an expected length of a contraction-relaxation cycle of the engineered tissue;
determine a predicted contraction type of a plurality of contraction types for the first waveform, wherein the plurality of contraction types include a single contraction type and a double contraction type;
fit a model to the first waveform based on the predicted contraction type, wherein the model parameterizes growth of the at least one contraction response of the engineered tissue independently of growth of the at least one relaxation response of the engineered tissue; and
generate a second waveform from the model fit to the first waveform such that the second waveform comprises a noise filtered representation of the first waveform.

41. A system comprising:
one or more processors; and
a memory storing computing instructions which, when executed by the one or more processors, cause the one or more processors to:
obtain a first waveform comprising at least one contraction response and at least one relaxation response of an engineered tissue, the first waveform having a predetermined length corresponding to an expected length of a contraction-relaxation cycle of the engineered tissue;
determine a predicted contraction type of a plurality of contraction types for the first waveform, wherein the plurality of contraction types include a single contraction type and a double contraction type;
fit a model to the first waveform based on the predicted contraction type, wherein the model parameterizes growth of the at least one contraction response of the engineered tissue independently of growth of the at least one relaxation response of the engineered tissue; and
generate a second waveform from the model fit to the first waveform such that the second waveform comprises a noise filtered representation of the first waveform.

42. A method for training a classifier to predict a tissue contraction type from a functional response waveform, the method comprising:
obtaining, by one or more processors, a plurality of waveforms comprising functional responses of a one or more engineered tissues, each of the plurality of waveforms having a predetermined length corresponding to an expected length of a contraction-relaxation cycle of an engineered tissue;
extracting, by the one or more processors, a first plurality of parameter sets from a first subset of the plurality of waveforms associated with a single contraction type, wherein a first parameter set of the first plurality of parameter sets characterizes a first waveform of the first subset of waveforms;
extracting, by the one or more processors, a second plurality of parameter sets from a second subset of the plurality of waveforms associated with a double contraction type, wherein a second parameter set of the second plurality of parameter sets characterizes a second waveform of the second subset of waveforms;
determining, by the one or more processors, a plurality of parameter set distributions, wherein the plurality of parameter set distributions comprise a first parameter set distribution determined from the first plurality of parameter sets and a second parameter set distribution determined from the second plurality of parameter sets;
generating, by the one or more processors, a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and
training, by the one or more processors, the classifier using the synthetic training data set, wherein the classifier trained using the synthetic training data set determines a predicted tissue contraction type for an input waveform.

43. The method of claim 42 wherein the classifier comprises a machine learning model.

44. The method of claim 43 wherein the machine learning model comprises a convolutional neural network.

45. The method of claim 42 further comprising:
obtaining, by the one or more processors, a first waveform comprising a functional response of a first artificial tissue, the first waveform having a length corresponding to an expected length of a contraction-relaxation cycle of the first artificial tissue; and
predicting, by the one or more processors and using the classifier trained on the synthetic training data set, a tissue contraction type from the first waveform.

46. The method of claim 45 further comprising:
outputting, by the one or more processors, the tissue contraction type predicted using the classifier.

47. The method of claim 42 wherein the first parameter set comprises a first plurality of parameter values of a first model, wherein the first model comprises a rising logistic function having a positive growth rate and a falling logistic function having a negative growth rate.

48. The method of claim 47 wherein the first plurality of parameter values comprises a maximum value parameter value, a rate of rise parameter value, a rate of fall parameter value, a y-shift parameter value, a rising x-shift parameter value, and a falling x-shift parameter value.

49. The method of claim 42 wherein the second parameter set comprises a second plurality of parameter values of a second model, wherein the second model comprises a plurality of rising logistic functions having positive growth rates and a plurality of first falling logistic function having negative growth rates.

50. The method of claim 49 wherein the second plurality of parameter values comprise a plurality of maximum value parameter values, a plurality of rate of rise parameter values, a plurality of rate of fall parameter values, a plurality of y-shift parameter values, a plurality of rising x-shift parameter values, and a plurality of falling x-shift parameter values.

51. The method of claim 42 wherein the one or more engineered tissues comprise one or more engineered muscle tissues.

52. The method of claim 51 wherein the one or more engineered muscle tissues are engineered cardiac tissues.

53. The method of claim 51 wherein the one or more engineered muscle tissues are engineered skeletal muscle tissues.

54. A non-transitory computer readable medium storing computing instructions which, when executed by a device comprising one or more processors, cause the one or more processors to:
obtain a plurality of waveforms comprising functional responses of a one or more engineered tissues, each of the plurality of waveforms having a predetermined length corresponding to an expected length of a contraction-relaxation cycle of an engineered tissue;
extract a first plurality of parameter sets from a first subset of the plurality of waveforms associated with a single contraction type, wherein a first parameter set of the first plurality of parameter sets characterizes a first waveform of the first subset of waveforms;
extract a second plurality of parameter sets from a second subset of the plurality of waveforms associated with a double contraction type, wherein a second parameter set of the second plurality of parameter sets characterizes a second waveform of the second subset of waveforms;
determine a plurality of parameter set distributions, wherein the plurality of parameter set distributions comprise a first parameter set distribution determined from the first plurality of parameter sets and a second parameter set distribution determined from the second plurality of parameter sets;
generate a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and
train a classifier using the synthetic training data set, wherein the classifier trained using the synthetic training data set determines a predicted tissue contraction type for an input waveform.

55. A system comprising:
one or more processors; and
a memory storing computing instructions which, when executed by the one or more processors, cause the one or more processors to:
obtain a plurality of waveforms comprising functional responses of a one or more engineered tissues, each of the plurality of waveforms having a predetermined length corresponding to an expected length of a contraction-relaxation cycle of an engineered tissue;
extract a first plurality of parameter sets from a first subset of the plurality of waveforms associated with a single contraction type, wherein a first parameter set of the first plurality of parameter sets characterizes a first waveform of the first subset of waveforms;
extract a second plurality of parameter sets from a second subset of the plurality of waveforms associated with a double contraction type, wherein a second parameter set of the second plurality of parameter sets characterizes a second waveform of the second subset of waveforms;
determine a plurality of parameter set distributions, wherein the plurality of parameter set distributions comprise a first parameter set distribution determined from the first plurality of parameter sets and a second parameter set distribution determined from the second plurality of parameter sets;
generate a synthetic training data set, each element of the synthetic training data set comprising a synthetic waveform and a corresponding tissue contraction type associated with the synthetic waveform, wherein the synthetic waveform is generated using a parameter set distribution of the plurality of parameter set distributions associated with the corresponding tissue contraction type; and
train a classifier using the synthetic training data set, wherein the classifier trained using the synthetic training data set determines a predicted tissue contraction type for an input waveform.

56. A method for processing a functional response waveform the method comprising:
obtaining, by one or more processors, a first waveform comprising a plurality of functional responses of an artificial tissue stimulated at a first frequency;
convolving, by the one or more processors, the first waveform with a pulse-train to generate a convolved waveform, wherein the pulse-train is generated at the first frequency;
identifying, by the one or more processors, a first location associated with a first maximum value of the convolved waveform, wherein the first location corresponds to an expected location of a first contraction-relaxation cycle; and
extracting, by the one or more processors and from the first location of the first waveform, a second waveform comprising the first contraction-relaxation cycle, wherein the second waveform has a first duration proportional to the first frequency.

57. The method of claim 56 further comprising:
outputting, by the one or more processors, the second waveform.

58. The method of claim 57 further comprising:
identifying, by the one or more processors, a second location associated with a second maximum value of the convolved waveform, wherein the second location corresponds to an expected location of a second contraction-relaxation cycle; and
extracting, by the one or more processors and from the second location of the first waveform, a third waveform comprising the second contraction-relaxation cycle, wherein the second waveform has a second duration proportional to the first frequency.

59. The method of claim 58 further comprising:
outputting, by the one or more processors, the third waveform.

60. The method of claim 58 wherein the first duration and the second duration are the same.

61. The method of claim 56 further comprising:
generating, by the one or more processors, the pulse-train at the first frequency.

62. The method of claim 56 wherein a midpoint of the second waveform is substantially aligned to the first location of the first waveform.

63. The method of claim 56 wherein the first frequency is from 0.1 Hz to 20 Hz.

64. The method of claim 63 wherein the first frequency is from 1 Hz to 6 Hz.

65. The method of claim 56 wherein the first waveform is obtained from a bioreactor comprising the artificial tissue.

66. The method of claim 56 wherein the artificial tissue comprises engineered muscle tissue.

67. The method of claim 66 wherein the engineered muscle tissue is engineered cardiac tissue.

68. The method of claim 67 wherein the engineered muscle tissue is engineered skeletal muscle tissue.

69. A non-transitory computer readable medium storing computing instructions which, when executed by a device comprising one or more processors, cause the one or more processors to:
obtain a first waveform comprising a plurality of functional responses of an artificial tissue stimulated at a first frequency;
convolve the first waveform with a pulse-train to generate a convolved waveform, wherein the pulse-train is generated at the first frequency;
identify a first location associated with a first maximum value of the convolved waveform, wherein the first location corresponds to an expected location of a first contraction-relaxation cycle; and
extract, from the first location of the first waveform, a second waveform comprising the first contraction-relaxation cycle, wherein the second waveform has a first duration proportional to the first frequency.

70. A system comprising:
one or more processors; and
a memory storing computing instructions which, when executed by the one or more processors, cause the one or more processors to:
obtain a first waveform comprising a plurality of functional responses of an artificial tissue stimulated at a first frequency;
convolve the first waveform with a pulse-train to generate a convolved waveform, wherein the pulse-train is generated at the first frequency;
identify a first location associated with a first maximum value of the convolved waveform, wherein the first location corresponds to an expected location of a first contraction-relaxation cycle; and
extract, from the first location of the first waveform, a second waveform comprising the first contraction-relaxation cycle, wherein the second waveform has a first duration proportional to the first frequency.

71. A method for predicting a perturbation effect, the method comprising:
obtaining, by one or more processors, a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions;
splitting, by the one or more processors, the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue;
determining, by the one or more processors, a predicted contraction type of a plurality of contraction types for each of the first plurality of waveforms, wherein the plurality of contraction types include a single contraction type and a double contraction type;
fitting, by the one or more processors, a model to each waveform of the first plurality of waveforms based on a corresponding predicted contraction type, wherein the model parameterizes growth of the at least one contraction response independently of growth of the at least one relaxation response of the engineered tissue;
generating, by the one or more processors, a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal;
extracting, by the one or more processors, a first feature value of a first feature from the plurality of filtered baseline waveforms;
extracting, by the one or more processors, a second feature value of the first feature from the plurality of filtered perturbation waveforms; and
determining, by the one or more processors, an effect associated with the first set of perturbation conditions based on a comparison of the first feature value and the second feature value.

72. The method of claim 71 wherein the first feature is one of a total number of single contraction type waveforms and a total number of double contraction type waveforms.

73. The method of claim 71 wherein the first feature is one of a contraction-relaxation cycle amplitude, a contraction time, a maximum contraction slope, a relaxation time, a maximum relaxation slope, and a contraction-relaxation cycle duration.

74. The method of claim 73 wherein the first feature value comprises an average value of the first feature determined from the plurality of filtered baseline waveforms.

75. The method of claim 73 wherein the second feature value comprises an average value of the first feature determined from the plurality of filtered perturbation waveforms.

76. The method of claim 71 further comprising:
outputting, by the one or more processors, the effect associated with a first compound.

77. The method of claim 71 wherein the step of fitting the model comprises fitting a first model for waveforms associated with the single contraction type.

78. The method of claim 77 wherein the first model comprises a first rising logistic function having a positive growth rate and a first falling logistic function having a negative growth rate.

79. The method of claim 78 wherein the first model comprises a product of the first rising logistic function and the first falling logistic function.

80. The method of claim 77 wherein the step of fitting the model comprises fitting the first model and a second model for waveforms associated with the double contraction type

81. The method of claim 80 wherein the second model comprises a second rising logistic function having a positive growth rate and a second falling logistic function having a negative growth rate.

82. The method of claim 81 wherein the second model comprises a product of the second rising logistic function and the second falling logistic function.

83. The method of claim 71 wherein the model comprises a plurality of parameters associated with the at least one contraction response and the at least one relaxation response.

84. The method of claim 83 wherein the plurality of parameters comprise at least one maximum value parameter, at least one rate of rise parameter, at least one rate of fall parameter, at least one y-shift parameter, at least one rising x-shift parameter, and at least one falling x-shift parameter.

85. The method of claim 83 wherein the step of fitting the model to a first waveform of the first plurality of waveforms comprises:
predicting, by the one or more processors, a plurality of values for the plurality of parameters of the model such that the model fit to the first waveform comprises the plurality of values for the plurality of parameters.

86. The method of claim 85 wherein the plurality of values are predicted using a machine learning model.

87. The method of claim 86 wherein the machine learning model comprises a trained inception model.

88. The method of claim 86 wherein the machine learning model comprises a trained convolutional neural network with dilated convolution.

89. The method of claim 86 wherein the machine learning model comprises a trained long short-term memory neural network.

90. The method of claim 85 wherein the step of fitting the model to the first waveform further comprises:
optimizing, by the one or more processors, the plurality of values by minimizing an error between the first waveform and the model fit to the first waveform using the plurality of values for the plurality of parameters of the model.

91. The method of claim 90 wherein the plurality of values are optimized using a simplex search algorithm.

92. The method of claim 71 wherein the predetermined length is from 0.05 s to 10 s.

93. The method of claim 92 wherein the predetermined length is from 0.15 s to 1 s.

94. The method of claim 71 wherein the at least one contraction response and the at least one relaxation response correspond to a functional response of the engineered tissue stimulated at a first frequency.

95. The method of claim 94 wherein the predetermined length is proportional to the first frequency.

96. The method of claim 71 wherein the predicted contraction type is determined by a classifier.

97. The method of claim 96 wherein the classifier comprises a trained convolutional neural network.

98. The method of claim 71 wherein the plurality of signals are obtained from a bioreactor comprising the engineered tissue.

99. The method of claim 71 wherein the engineered tissue comprises engineered muscle tissue.

100. The method of claim 99 wherein the engineered muscle tissue is engineered cardiac tissue.

101. The method of claim 99 wherein the engineered muscle tissue is engineered skeletal muscle tissue.

102. The method of claim 71 wherein the control conditions are associated with the engineered tissue having been vehicle treated.

103. The method of claim 71 wherein the first set of perturbation conditions are associated with one or more of: a treatment of the engineered tissue with a drug or compound; a different cell line; a disease state of the engineered tissue; a physical perturbation of the engineered tissue; and changes to an environment of the engineered tissue.

104. A non-transitory computer readable medium storing computing instructions which, when executed by a device comprising one or more processors, cause the one or more processors to:
obtain a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions;
split the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue;
determine a predicted contraction type of a plurality of contraction types for each of the first plurality of waveforms, wherein the plurality of contraction types include a single contraction type and a double contraction type;
fit a model to each waveform of the first plurality of waveforms based on a corresponding predicted contraction type, wherein the model parameterizes growth of the at least one contraction response independently of growth of the at least one relaxation response of the engineered tissue;
generate a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal;
extract a first feature value of a first feature from the plurality of filtered baseline waveforms;
extract a second feature value of the first feature from the plurality of filtered perturbation waveforms; and
determine an effect associated with the first set of perturbation conditions based on a comparison of the first feature value and the second feature value.

105. A system comprising:
one or more processors; and
a memory storing computing instructions which, when executed by the one or more processors, cause the one or more processors to:
obtain a plurality of signals comprising a baseline signal and a perturbation signal, wherein the baseline signal comprises a first plurality of functional responses of an engineered tissue under control conditions and the perturbation signal comprises a second plurality of functional responses of the engineered tissue under a first set of perturbation conditions;
split the plurality of signals into a first plurality of waveforms, each waveform of the first plurality of waveforms having a predetermined length and comprising at least one contraction response and at least one relaxation response of the engineered tissue, wherein the predetermined length corresponds to an expected length of a contraction-relaxation cycle of the engineered tissue;
determine a predicted contraction type of a plurality of contraction types for each of the first plurality of waveforms, wherein the plurality of contraction types include a single contraction type and a double contraction type;
fit a model to each waveform of the first plurality of waveforms based on a corresponding predicted contraction type, wherein the model parameterizes growth of the at least one contraction response independently of growth of the at least one relaxation response of the engineered tissue;
generate a second plurality of waveforms from the model fit to each waveform of the first plurality of waveforms, wherein the second plurality of waveforms comprise a plurality of filtered baseline waveforms associated with the baseline signal and a plurality of filtered perturbation waveforms associated with the perturbation signal;
extract a first feature value of a first feature from the plurality of filtered baseline waveforms;
extract a second feature value of the first feature from the plurality of filtered perturbation waveforms; and
determine an effect associated with the first set of perturbation conditions based on a comparison of the first feature value and the second feature value.

106. A method for training a model using synthetic training data, the method comprising:
obtaining, by one or more processors, a plurality of waveforms comprising functional responses of one or more artificial tissues, wherein the plurality of waveforms all comprise a common contraction type of a predetermined plurality of contraction types;
each of the plurality of waveforms having a predetermined length corresponding to an expected length of a contraction-relaxation cycle of an artificial tissue, of the plurality of waveforms have a same contraction type;
extracting, by the one or more processors, a plurality of parameter sets from the plurality of waveforms, wherein a parameter set of the plurality of parameter sets characterizes a corresponding waveform of the plurality of waveforms;
determining, by the one or more processors, a parameter set distribution from the plurality of parameter sets;
generating, by the one or more processors, a synthetic training data set, each element of the synthetic training data set comprising a synthetic waveform and a corresponding parameter set used to generate the synthetic waveform, wherein the corresponding parameter set is obtained from the parameter set distribution; and
training, by the one or more processors, a parameter estimation model using the synthetic training data set, wherein the parameter estimation model is trained to estimate an output parameter set from an input waveform.

107. The method of claim 106 wherein the parameter estimation model comprises an inception model.

108. The method of claim 106 wherein the parameter estimation model comprises a convolutional neural network with dilated convolution.

109. The method of claim 106 wherein the parameter estimation model comprises a long short-term memory neural network.

110. The method of claim 106 further comprising:
obtaining, by the one or more processors, a first waveform comprising a functional response of a first artificial tissue; and
predicting, by the one or more processors and using the parameter estimation model trained on the synthetic training data set, a first set of parameter values from the first waveform.

111. The method of claim 110 further comprising:
outputting, by the one or more processors, the first set of parameter values.

112. The method of claim 106 wherein the step of generating the synthetic training data set further comprises:
adding, by the one or more processors, a noise component to the synthetic waveform of each element of the synthetic training data set according to a noise model.

113. The method of claim 112 wherein the noise model is determined from the plurality of waveforms.

114. The method of claim 106 wherein the parameter set comprises parameter values of a model comprising at least one rising logistic function having a positive growth rate and at least one falling logistic function having a negative growth rate.

115. The method of claim 114 wherein the parameter values comprise at least one maximum value parameter value, at least one rate of rise parameter value, at least one rate of fall parameter value, a y-shift parameter value, at least one rising x-shift parameter value, and at least one falling x-shift parameter value.

116. The method of claim 106 wherein the one or more artificial tissues comprise one or more engineered muscle tissues.

117. The method of claim 116 wherein the one or more engineered muscle tissues are engineered cardiac tissues.

118. The method of claim 116 wherein the one or more engineered muscle tissues are engineered skeletal muscle tissues.

119. The method of claim 106 wherein the predetermined plurality of contraction types include a single contraction type and a double contraction type.

120. A non-transitory computer readable medium storing computing instructions which, when executed by a device comprising one or more processors, cause the one or more processors to:
obtain a plurality of waveforms comprising functional responses of one or more artificial tissues during a single contraction-relaxation cycle;
extract a plurality of parameter sets from the plurality of waveforms, wherein a parameter set of the plurality of parameter sets characterizes a corresponding waveform of the plurality of waveforms;
determine a parameter set distribution from the plurality of parameter sets;
generate a synthetic training data set, each element of the synthetic training data set comprising a synthetic waveform and a corresponding parameter set used to generate the synthetic waveform, wherein the corresponding parameter set is obtained from the parameter set distribution; and
train a parameter estimation model using the synthetic training data set, wherein the parameter estimation model is trained to estimate an output parameter set from an input waveform.

121. A system comprising:
one or more processors; and
a memory storing computing instructions which, when executed by the one or more processors, cause the one or more processors to:
obtain a plurality of waveforms comprising functional responses of one or more artificial tissues during a single contraction-relaxation cycle;
extract a plurality of parameter sets from the plurality of waveforms, wherein a parameter set of the plurality of parameter sets characterizes a corresponding waveform of the plurality of waveforms;
determine a parameter set distribution from the plurality of parameter sets;
generate a synthetic training data set, each element of the synthetic training data set comprising a synthetic waveform and a corresponding parameter set used to generate the synthetic waveform, wherein the corresponding parameter set is obtained from the parameter set distribution; and
train a parameter estimation model using the synthetic training data set, wherein the parameter estimation model is trained to estimate an output parameter set from an input waveform.
